(19) United States
(12) Patent Application Publication
(10) Pub. No.: US 2013/0281429 A1
(43) Pub. Date: Oct. 24, 2013

(54) 2,4-DIAMINO-PYRIMIDINES AS AURORA INHIBITORS

(71) Applicants: Stephan Karl ZAHN, Vienna (AT); Guido BOEHMELT, Gaaden (AT); Andreas MANTOULIDIS, Vienna (AT); Ulrich REISER, Vienna (AT); Matthias TREU, Vienna (AT); Ulrich GUERTLER, Opfingen (DE); Andreas SCHOOP, Neuried (DE); Flavio SOLCA, Vienna (AT); Ulrike TONTSCH-GRUNT, Baden (AT); Ralph BRUECKNER, Hallstadt (DE); Charlotte REITHER, Vienna (AT); Lars HERFURTH, Appenheim (DE); Oliver KRAEMER, Vienna (AT); Heinz STADTMUELLER, Vienna (AT); Harald ENGELHARDT, Ebreichsdorf (AT)

(72) Inventors: Stephan Karl ZAHN, Vienna (AT); Guido BOEHMELT, Gaaden (AT); Andreas MANTOULIDIS, Vienna (AT); Ulrich REISER, Vienna (AT); Matthias TREU, Vienna (AT); Ulrich GUERTLER, Opfingen (DE); Andreas SCHOOP, Neuried (DE); Flavio SOLCA, Vienna (AT); Ulrike TONTSCH-GRUNT, Baden (AT); Ralph BRUECKNER, Hallstadt (DE); Charlotte REITHER, Vienna (AT); Lars HERFURTH, Appenheim (DE); Oliver KRAEMER, Vienna (AT); Heinz STADTMUELLER, Vienna (AT); Harald ENGELHARDT, Ebreichsdorf (AT)

(73) Assignee: BOEHRINGER INGELHEIM INTERNATIONAL GMBH, INGELHEIM AM RHEIN (DE)

(21) Appl. No.: 13/850,711

(22) Filed: Mar. 26, 2013

Related U.S. Application Data
(63) Continuation of application No. 13/159,787, filed on Jun. 14, 2011, now abandoned.
Application No. 13/159,787 is a continuation of application No. 11/475,468, filed on Jun. 27, 2006, now abandoned.

(30) Foreign Application Priority Data
Jul. 1, 2005 (EP) 05106007.7

(51) Int. Cl. C07D 239/48; C07D 401/14; C07D 401/12; C07D 487/08; A61K 31/5377; A61K 45/06; A61K 31/506; A61K 31/505; A61K 31/551; C07D 403/12; C07D 409/14

(52) U.S. Cl. CPC C07D 239/48; C07D 403/12; C07D 401/14; C07D 401/12; C07D 487/08; C07D 409/14; A61K 45/06; A61K 31/506; A61K 31/505; A61K 31/551; A61K 31/5377
USPC 514/210.18; 544/323; 544/295; 540/575; 544/121; 544/324; 514/275; 514/252.14; 514/218; 514/235.8; 514/252.19; 514/252.18

(57) ABSTRACT

The present invention encompasses compounds of general formula (1)

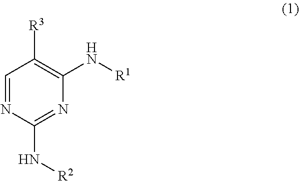

wherein
R¹ to R³ are defined as in claim 1, which are suitable for the treatment of diseases characterised by excessive or abnormal cell proliferation, and the use thereof for preparing a pharmaceutical composition having the above-mentioned properties.

The present invention relates to new 2,4-diamino-pyrimidines of general formula (1)

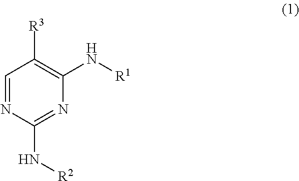

wherein the groups R¹ to R³ have the meanings given in the claims and specification, the isomers thereof, processes for preparing these pyrimidines and their use as pharmaceutical compositions.

### BACKGROUND TO THE INVENTION

Tumour cells wholly or partly elude regulation and control by the body and are characterised by uncontrolled growth. This is due on the one hand to the loss of control proteins such as for example Rb, p16, p21 and p53 and also to the activation of so-called accelerators of the cell cycle, the cyclin-dependent kinases.

Studies in model organisms such as Schizosaccharomyces pombe, Drosophila melanogaster or Xenopus as well as investigations in human cells have shown that the transition from the G2 phase to mitosis is regulated by the CDK1/cyclin B kinase (Nurse, 1990). This kinase, which is also known as “mitosis promoting factor” (MPF), phosphorylates and regulates a plurality of proteins, such as e.g. nuclear lamina, kinesin-like motor proteins, condensins and Golgi Matrix Proteins, which play an important part in the breakdown of the nuclear coat, in centrosome separation, the structure of the mitotic spindle apparatus, chromosome condensation and breakdown of the Golgi apparatus (Nigg, 2001). The treatment of human tumour cells with inhibitors against CDK1/cyclin B, such as e.g. butyrolactone, leads to an arrest in the G2/M phase and subsequent apoptosis (Nishio, et al. 1996).

In addition to the cyclin-dependent kinases the so-called polo-like serine/threonine kinases (PLK-1, PLK-2, PLK-3 and PLK-4) play an important role in the regulation of the eukaryotic cell cycle. PLK-1 in particular has been found to play a central role in the regulation of the mitosis phase. PLK-1 is responsible for the maturation of the centrosomes, for the activation of phosphatase Cdc25C, as well as for the activation of the Anaphase Promoting Complex (Glover et al., 1998, Qian et al., 2001). The injection of PLK-1 antibodies leads to a G2 arrest in untransformed cells, whereas tumour cells arrest during the mitosis phase (Lane and Nigg, 1996).

Moreover, an arrest in the G2/M phase may also be initiated by inhibition of specific motor proteins, the so-called kinesins such as for example Eg5 (Mayer et al., 1999), or by microtubuli stabilising or destabilising agents (e.g. colchicin, taxol, etoposide, vinblastine, vincristine) (Schiff and Horwitz, 1980).

The Ser/Thr kinases of the Aurora family regulate various processes of cell division. These include chromosome condensation, spindle dynamics, kinetochor-microtubule interactions, chromosome orientation, the alignment of the metaphasis plate and cytokinesis (Meraldi et al., 2004; Carmena and Earnshaw, 2003; Andrews et al., 2003). Three members of the family have been described in mammals—Aurora A, B and C. Aurora kinases of the A- and B-type also exist in Caenorhabditis elegans and Drosophila melanogaster, whereas yeasts contain only a single Aurora gene which is known by the name IPL1 (in S. cerevisiae), or ARK1 (in S. pombe). All the Aurora proteins share a similar overall structure which comprises a variable N-terminus, a well conserved central kinase domain and a short C-terminal part. In spite of the similarity of their sequences the kinases of the Aurora family exhibit different subcellular localisation which is linked to specialised functions.

Thus, Aurora A is to be found in the interphase in centrosomes and during mitosis both on centrosomes and on spindle microtubuli close to the poles. Accordingly—as confirmed by RNA interference experiments—Aurora A is essential for entry into mitosis, as centrosome maturation and separation cannot take place when Aurora A is lost. There are various activators for Aurora A, such as e.g. TPX2, Ajuba or protein phosphatase inhibitor-2. TPX2 appears to be responsible for the correct activation of Aurora A in time and space on spindle microtubuli close to the pole (Hirota et al., 2003; Bayliss et al., 2003; Eyers and Maller, 2004; Kufer et al., 2002; Satinover et al., 2004).

Aurora B associates in the early prophase with condensing chromosomes, locates in the metaphase on centromeres, re-locates thereafter in the central zone of the central spindle and then finally becomes concentrated at the moment of cytokinesis on the so-called Flemming or central body, a narrowly defined region between the daughter cells. These characteristic spatial changes during mitosis justify referring to Aurora B as a so-called “chromosomal passenger” protein. At least three other “chromosomal passenger” proteins are known which form a complex with Aurora B. They are INCENP (inner centromere protein), survivin and borealin (Andrews et al., 2003; Carmena and Earnshaw, 2003; Meraldi et al., 2004). An important point of contact between Aurora B and this complex is provided by the C-terminus of INCENP, the so-called “IN-box”. The “IN-box” is the most highly conserved region of INCENP. It binds and activates Aurora B and is phosphorylated by this kinase (Adams et al., 2000; Bishop and Schumacher, 2002; Kaitna et al., 2000; Bolton et al., 2002; Honda et al., 2003).

Aurora C is the least characterised member of the Aurora family. Aurora C also binds to INCENP and behaves as a “chromosomal passenger” protein, although after Aurora B it has the highest expression levels. Aurora C is presumably able to take over some functions from Aurora B, as for example the polynuclear phenotype Aurora B—depleted cells can be normalised by the expression of Aurora C (Sasai et al., 2004; Li et al., 2004).

Aurora B phosphorylates histone H3 at Ser10 and Ser28. Although this phosphorylation coincides with the moment of chromosome condensation, the effect of this event is only relevant at a later stage of the cell cycle. This is confirmed by the fact that histone H3 is concentrated in mitotic chromosomes with Ser10 phosphorylation and simultaneous Lys9 triple methylation on heterochromatin near the centromere. Histone H3 thus modified prevents the binding of heterochromatin protein 1 (HP1) and permits access to centromeric kinetochore regions by the “chromosomal passenger” protein complex (Hirota T. et al., Manuscript in Preparation).

One function of Aurora B, which is made obvious by the inhibition of Aurora B, is in the combining of different proteins on the kinetochore during the metaphase (Ditchfield et al., 2003; Hauf et al., 2003; Murata-Hori and Wang, 2002; Vigneron et al., 2004). Aurora B plays a central role in a signal pathway which detects and corrects syntelic (defective, because they are starting from only one spindle pole) kinetochore attachments of microtubules (Andrews et al., 2003; Carmena and Earnshaw, 2003; Meraldi et al., 2004). If this state of attachment is not corrected, errors occur in chromosome segregation. The Aurora B-mediated phosphorylation of the microtubule depolymerase MCAK is linked to this correction mechanism (Gorbsky, 2004).

Aurora B also phosphorylates proteins which are important for forming the replication form and cytokinesis, such as e.g. MgcRacGAP, the light regulatory chain of myosin II, vimentin, desmin, GFAP (glial fibrillary acidic protein), as well as the kinesins MKLP1 and MKLP2, of which MKLP2 is presumably responsible for completing the transfer of the “chromosomal passenger” protein complex from the kinetochores to the central body (Gruneberg et al., 2004).

In view of the various functions of Aurora B in the cell cycle, it is was surprising to find that inhibiting Aurora B in tumour cells does not cause mitotic arrest but rather continuation of the cell cycle without cytokinesis (Hauf et al., 2003). As a result of the accumulation of syntelic microtubule-kinetochore attachments and therefore faulty chromosome segregations, massive polyploidia occurs, finally leading to apoptosis. Even the simultaneous inhibition of Aurora A cannot influence this phenotype (Keen and Taylor, 2004).

Initially there were predominantly indications of the oncogenic activity of Aurora A (e.g. transformation of murine fibroblasts after overexpression), whereas for Aurora B such indications were only indirectly present (Zhou et al., 1998; Bischoff et al., 1998; Katayama et al., 1999). This changed with the finding that overexpression of Aurora B in embryonic hamster cells and the use thereof in xenograft experiments directly increases the incidence, size and invasiveness of tumours. Corresponding tumours exhibited chromosomal instability and increased histone H3 Ser10 phosphorylation (Ota et al., 2002). These results underpin the importance of Aurora B during tumour genesis.

Pyrimidines are generally known as inhibitors of kinases. Thus, for example, substituted pyrimidines with a non-aromatic group in the 4-position as active components with anti-cancer effects are described in International patent applications WO 02/096888 and WO 03/032997. The aim of the present invention is to indicate new active substances which can be used for the prevention and/or treatment of diseases characterised by excessive or abnormal cell proliferation.

### DETAILED DESCRIPTION OF THE INVENTION

It has now been found that, surprisingly, compounds of general formula (1), wherein the groups R¹, R² and R³ are defined as hereinafter, act as inhibitors of specific cell cycle kinases. Thus, the compounds according to the invention may be used for example for the treatment of diseases associated with the activity of specific cell cycle kinases and characterised by excessive or anomalous cell proliferation.

The present invention relates to compounds of general formula (1)

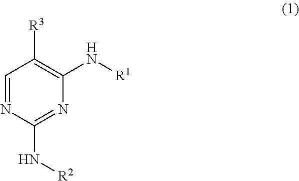

wherein

R¹ denotes a group, substituted by R⁵ and optionally by one or more R⁴, selected from among C₃₋₁₀-Cycloalkyl and 3-8-membered heterocycloalkyl;

R² denotes a group, optionally substituted by one or more R⁴, selected from among C₁₋₆-alkyl, C₃₋₁₀-Cycloalkyl, 3-8-membered heterocycloalkyl, C₆₋₁₅-aryl and 5-12-membered heteroaryl;

R³ denotes a group selected from among hydrogen, halogen, —CN, —NO₂, C₁₋₄-alkyl, C₁₋₄-haloalkyl, C₃₋₁₀-cycloalkyl, C₄₋₁₆-cycloalkylalkyl and C₇₋₁₆-arylalkyl;

R⁴ denotes a group selected from among R^{a}, R^{b} and R^{a} substituted by one or more identical or different R^{c} and/or R^{b};

R⁵ denotes a suitable group selected from among —C(O)R^{D}, —C(O)NR^{c}R^{c}, —S(O)₂R^{c}, —N(R^{f})S(O)₂R^{c}, —N(R^{f})C(O)R^{c}, —N(R^{f})C(O)OR^{c}, and —N(R^{f})C(O)NR^{c}R^{c};

each R^{a} is selected independently of one another from among C₁₋₆alkyl, C₃₋₁₀-cycloalkyl, C₄₋₁₆-cycloalkylalkyl, C₆₋₁₀aryl, C₇₋₁₆arylalkyl, 2-6-membered heteroalkyl, 3-8-membered heterocycloalkyl, 4-14-membered heterocycloalkylalkyl, 5-12-membered heteroaryl and 6-18-membered heteroarylalkyl;

each R^{b} is a suitable group and in each case selected independently of one another from among ═O, —OR^{c}, C₁₋₃haloalkyloxy, —OCF₃, ═S, —SR^{c}, ═NR^{c}, ═NOR^{c}, —NR^{c}R^{c}, halogen, —CF₃, —CN, —NC, —OCN, —SCN, —NO₂, —S(O)R^{c}, —S(O)₂R^{c}, —S(O)₂OR^{c}, —S(O)NR^{c}R^{c}, —S(O)₂NR^{c}R^{c}, —OS(O)R^{c}, —OS(O)₂R^{c}, —OS(O)₂OR^{c}, —OS(O)₂NR^{c}R^{c}, —C(O)R^{c}, —C(O)OR^{c}, —C(O)NR^{c}R^{c}, —CN(R^{f})NR^{c}R^{c}, —CN(OH)R^{c}, —CN(OH)NR^{c}R^{c}, —OC(O)R^{c}, —OC(O)OR^{c}, —OC(O)NR^{c}R^{c}, —OCN(R^{f})NR^{c}R^{c}, —N(R^{f})C(O)R^{c}, —N(R^{f})C(S)R^{c}, —N(R^{f})S(O)₂R^{c}, —N(R^{f})C(O)OR^{c}, —N(R^{f})C(O)NR^{c}R^{c}, —[N(R^{f})C(O)]₂R^{c}, —N[C(O)]₂R^{c}, —N[C(O)]₂OR^{c}, —[N(R^{f})C(O)]₂OR^{c} and —N(R^{f})CN(R^{f})NR^{c}R^{c};

each R^{c} independently of one another is hydrogen or a group optionally substituted by one or more identical or different R^{d} and/or R^{e} selected from among C₁₋₆-alkyl, C₃₋₁₀-cycloalkyl, C₄₋₁₁-cycloalkylalkyl, C₆₋₁₀aryl, C₇₋₁₆ arylalkyl, 2-6-membered heteroalkyl, 3-8-membered heterocycloalkyl, 4-14-membered heterocycloalkylalkyl, 5-12-membered heteroaryl and 6-18-membered heteroarylalkyl,

each R^{d} independently of one another is hydrogen or a group optionally substituted by one or more identical or different R^{e} and/or R^{f} selected from among C₁₋₆alkyl, C₃₋₈-cycloalkyl, C₄₋₁₁-cycloalkylalkyl, C₆₋₁₀aryl, C₇₋₁₆arylalkyl, 2-6-membered heteroalkyl, 3-8-membered heterocycloalkyl, 4-14-membered heterocycloalkylalkyl, 5-12-membered heteroaryl and 6-18-membered heteroarylalkyl;

each R^{e} is a suitable group and each selected independently of one another from among ═O, —OR^{f}, C₁₋₃haloalykloxy, —OCF₃, ═S, —SR^{f}, ═NR^{f}, ═NOR^{f}, —NR^{f}R^{f}, halogen, —CF₃, —CN, —NC, —OCN, —SCN, —NO₂, —S(O)R^{f}, —S(O)₂R^{f}, —S(O)₂OR^{f}, —S(O)NR^{f}R^{f}, —S(O)₂NR^{f}R^{f}, —OS(O)R^{f}, —OS(O)₂R^{f}, —OS(O)₂OR^{f}, —OS(O)₂NR^{f}R^{f}, —C(O)R^{f}, —C(O)OR^{f}, —C(O)NR^{f}R^{f}, —CN(R^{g})NR^{f}R^{f}, —CN(OH)R^{f}, —C(NOH)NR^{f}R^{f}, —OC(O)R^{f}, —OC(O)OR^{f}, —OC(O)NR^{f}R^{f}, —OCN(R^{g})NR^{f}R^{f}, —N(R^{g})C(O)R^{f}, —N(R^{g})C(S)R^{f}, —N(R^{g})S(O)₂R^{f}, —N(R^{d})C(O)OR^{f}, —N(R^{g})C(O)NR^{f}R^{f}, and —N(R^{g})CN(R^{f})NR^{f}R^{f};

each R^{f} independently of one another is hydrogen or a group optionally substituted by one or more identical or different R^{g} selected from among C₁₋₆alkyl, C₃₋₈-cycloalkyl, C₄₋₁₁-cycloalkylalkyl, C₆₋₁₀aryl, C₇₋₁₆ arylalkyl, 2-6-membered heteroalkyl, 3-8-membered heterocycloalkyl, 4-14-membered heterocycloalkylalkyl, 5-12-membered heteroaryl and 6-18-membered heteroarylalkyl;

each R^{g} independently of one another is hydrogen, C₁₋₆alkyl, C₃₋₈-cycloalkyl, C₄₋₁₁-cycloalkylalkyl, C₆₋₁₀aryl, C₇₋₁₆ arylalkyl, 2-6-membered heteroalkyl, 3-8-membered heterocycloalkyl, 4-14-membered heterocycloalkyl, 5-12-membered heteroaryl and 6-18-membered heteroarylalkyl, optionally in the form of the tautomers, the racemates, the enantiomers, the diastereomers and the mixtures thereof, and optionally the pharmacologically acceptable acid addition salts thereof.

In one aspect the invention relates to compounds of general formula (1), wherein R³ denotes a group selected from among halogen and C₁₋₄haloalkyl.

In another aspect the invention relates to compounds of general formula (1), wherein R³ denotes —CF₃.

In another aspect the invention relates to compounds of general formula (1), wherein R² denotes C₆₋₁₀aryl or 5-12-membered heteroaryl, optionally substituted by one or more R⁴.

In another aspect the invention relates to compounds of general formula (1), wherein R² denotes phenyl, optionally substituted by one or more R⁴.

In another aspect the invention relates to compounds of general formula (1A),

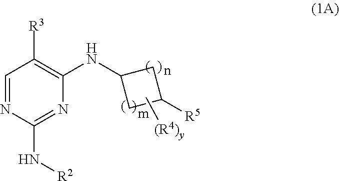

wherein

n is equal to 0 or 1, and

m is equal to 1-5, and

y is equal to 0 to 6, and the remaining groups are as hereinbefore defined.

In another aspect the invention relates to compounds of general formula (1A), wherein R³ denotes a group selected from among halogen and C₁₋₄haloalkyl.

In another aspect the invention relates to compounds of general formula (1A), wherein R³ denotes CF₃.

In another aspect the invention relates to compounds of general formula (1A), wherein R² denotes C₆₋₁₀aryl or 5-12-membered heteroaryl, optionally substituted by one or more R⁴.

In another aspect the invention relates to compounds of general formula (1A), wherein R² denotes phenyl, optionally substituted by one or more R⁴.

In another aspect the invention relates to compounds, or the pharmaceutically active salts thereof, of general formula (1) or (1A), for use as pharmaceutical compositions.

In another aspect the invention relates to compounds, or the pharmaceutically active salts thereof, of general formula (1) or (1A), for preparing a pharmaceutical composition with an antiproliferative activity.

In another aspect the invention relates to pharmaceutical preparations, containing as active substance one or more compounds of general formula (1) or (1A) or the physiologically acceptable salts thereof, optionally in conjunction with conventional excipients and/or carriers.

In another aspect the invention relates to the use of compounds of general formula (1) or (1A) for preparing a pharmaceutical composition for the treatment and/or prevention of cancer, infections, inflammatory and autoimmune diseases.

In another aspect the invention relates to pharmaceutical preparation comprising a compound of general formula (1) or (1A) and at least one other cytostatic or cytotoxic active substance, different from formula (1), optionally in the form of the tautomers, racemates, enantiomers, diastereomers and mixtures thereof, and optionally the pharmacologically acceptable acid addition salts thereof.

### DEFINITIONS

As used herein, the following definitions apply, unless stated otherwise.

By alkyl substituents are meant in each case saturated, unsaturated, straight-chain or branched aliphatic hydrocarbon groups (alkyl group) and the definition includes both saturated alkyl groups and unsaturated alkenyl and alkynyl groups. Alkenyl substituents are in each case straight-chain or branched, unsaturated alkyl groups which have at least one double bond. By alkynyl substituents are meant in each case straight-chain or branched, unsaturated alkyl groups which have at least one triple bond.

Heteroalkyl denotes straight-chain or branched aliphatic hydrocarbon chains which contain 1 to 3 heteroatoms, while each of the available carbon and heteroatoms in the heteroalkyl chain may each optionally be substituted independently of one another and the heteroatoms are selected independently of one another from the group consisting of O, N, P, PO, PO₂, S, SO and SO₂ (e.g. dimethylaminomethyl, dimethylaminoethyl, dimethylaminopropyl, diethylaminomethyl, diethylaminoethyl, diethylaminopropyl, 2-diisopropylaminoethyl, bis-2-methoxyethylamino, [2-(dimethylamino-ethyl)-ethyl-amino]-methyl, 3-[2-(dimethylamino-ethyl)-ethyl-amino]-propyl, hydroxymethyl, 2-hydroxyethyl, 3-hydroxypropyl, methoxy, ethoxy, propoxy, methoxymethyl, 2-methoxyethyl).

Haloalkyl refers to alkyl groups wherein one or more hydrogen atoms are replaced by halogen atoms. Haloalkyl includes both saturated alkyl groups and unsaturated alkenyl and alkynyl groups, such as for example —CF₃, —CHF₂, —CH₂F, —CF₂CF₃, —CHFCF₃, —CH₂CF₃, —CF₂CH₃, —CHFCH₃, —CF₂CF₂CF₃, —CF₂CH₂CH₃, —CF═CF₂, —CCl═CH₂, —CBr═CH₂, —CJ=CH₂, —C≡C—CF₃, —CHFCH₂CH₃ and —CHFCH₂CF₃.

Halogen refers to fluorine, chlorine, bromine and/or iodine atoms.

By cycloalkyl is meant a mono- or polycyclic ring, wherein the ring system may be a saturated ring but also an unsaturated, non-aromatic ring or a spiro compound, which may optionally also contain double bonds, such as for example cyclopropyl, cyclopropenyl, cyclobutyl, cyclobutenyl, cyclopentyl, cyclopentenyl, cyclohexyl, cyclohexenyl, cycloheptanyl, cycloheptenyl, norbornyl, norbornenyl, indanyl, adamantyl, spiroheptanyl and spiro[4.2]heptanyl.

Cycloalkylalkyl includes a non-cyclic alkyl group wherein a hydrogen atom bound to a carbon atom is replaced by a cycloalkyl group.

Aryl relates to monocyclic or bicyclic rings with 6-12 carbon atoms such as for example phenyl and naphthyl.

Arylalkyl includes a non-cyclic alkyl group wherein a hydrogen atom bound to a carbon atom is replaced by an aryl group.

By heteroaryl are meant mono- or polycyclic rings which contain, instead of one or more carbon atoms, one or more heteroatoms, which may be identical or different, such as e.g. nitrogen, sulphur or oxygen atoms. Examples include furyl, thienyl, pyrrolyl, oxazolyl, thiazolyl, isoxazolyl, isothiazolyl, pyrazolyl, imidazolyl, triazolyl, tetrazolyl, oxadiazolyl, thiadiazolyl, pyridyl, pyrimidyl, pyridazinyl, pyrazinyl and triazinyl. Examples of bicyclic heteroaryl groups are indolyl, isoindolyl, benzofuranyl, benzothienyl, benzoxazolyl, benzothiazolyl, benzisoxazolyl, benzisothiazolyl, benzimidazolyl, indazolyl, isoquinolinyl, quinolinyl, quinoxalinyl, cinnolinyl, phthalazinyl, quinazolinyl and benzotriazinyl, indolizinyl, oxazolopyridinyl, imidazopyridinyl, naphthyridinyl, indolinyl, isochromanyl, chromanyl, tetrahydroisochinolinyl, isoindolinyl, isobenzotetrahydrofuranyl, isobenzotetrahydrothienyl, isobenzothienyl, benzoxazolyl, pyridopyridinyl, benzotetrahydrofuranyl, benzotetrahydrothienyl, purinyl, benzodioxolyl, triazinyl, phenoxazinyl, phenothiazinyl, pteridinyl, benzothiazolyl, imidazopyridinyl, imidazothiazolyl, dihydrobenzisoxazinyl, benzisoxazinyl, benzoxazinyl, dihydrobenzisothiazinyl, benzopyranyl, benzothiopyranyl, coumarinyl, isocoumarinyl, chromonyl, chromanonyl, pyridinyl-N-oxide, tetrahydroquinolinyl, dihydroquinolinyl, dihydroquinolinonyl, dihydroisoquinolinonyl, dihydrocoumarinyl, dihydroisocoumarinyl, isoindolinonyl, benzodioxanyl, benzoxazolinonyl, pyrrolyl-N-oxide, pyrimidinyl-N-oxide, pyridazinyl-N-oxide, pyrazinyl-N-oxide, quinolinyl-N-oxide, indolyl-N-oxide, indolinyl-N-oxide, isoquinolyl-N-oxide, quinazolinyl-N-oxide, quinoxalinyl-N-oxide, phthalazinyl-N-oxide, imidazolyl-N-oxide, isoxazolyl-N-oxide, oxazolyl-N-oxide, thiazolyl-N-oxide, indolizinyl-N-oxide, indazolyl-N-oxide, benzothiazolyl-N-oxide, benzimidazolyl-N-oxide, pyrrolyl-N-oxide, oxadiazolyl-N-oxide, thiadiazolyl-N-oxide, triazolyl-N-oxide, tetrazolyl-N-oxide, benzothiopyranyl-5-oxide and benzothiopyranyl-S,S-dioxide.

Heteroarylalkyl encompasses a non-cyclic alkyl group wherein a hydrogen atom bound to a carbon atom is replaced by a heteroaryl group.

Heterocyclyl relates to saturated or unsaturated, non-aromatic mono-, bicyclic or bridged polycyclic rings or spiro compounds comprising 3-12 carbon atoms, which carry heteroatoms, such as nitrogen, oxygen or sulphur, instead of one or more carbon atoms. Examples of such heterocylyl groups are tetrahydrofuranyl, pyrrolidinyl, pyrrolinyl, imidazolidinyl, imidazolinyl, pyrazolidinyl, pyrazolinyl, piperidinyl, piperazinyl, indolinyl, isoindolinyl, morpholinyl, thiomorpholinyl, homomorpholinyl, homopiperidinyl, homopiperazinyl, homothiomorpholinyl, thiomorpholinyl-5-oxide, thiomorpholinyl-S,S-dioxide, tetrahydropyranyl, tetrahydrothienyl, homothiomorpholinyl-S,S-dioxide, oxazolidinonyl, dihydropyrazolyl, dihydropyrrolyl, dihydropyrazinyl, dihydropyridinyl, dihydropyrimidinyl, dihydrofuryl, dihydropyranyl, tetrahydrothienyl-5-oxide, tetrahydrothienyl-S,S-dioxide, homothiomorpholinyl-5-oxide, 2-oxa-5-azabicyclo[2.2.1]heptane, 8-oxa-3-aza-bicyclo[3.2.1]octane, 3,8-diaza-bicyclo[3.2.1]octane, 2,5-diaza-bicyclo[2.2.1]heptane, 3,8-diaza-bicyclo[3.2.1]octane, 3,9-diaza-bicyclo[4.2.1]nonane and 2,6-diaza-bicyclo[3.2.2]nonane.

Heterocycloalkylalkyl relates to a non-cyclic alkyl group wherein a hydrogen atom bound to a carbon atom is replaced by a heterocycloalkyl group.

### LIST OF ABBREVIATIONS

| Eq., eq | Equivalent(s) | IR | Infrared spectroscopy |
|---|---|---|---|
| Ac | acetyl | Cat., cat | catalyst, catalytic |
| Boc | t-butyloxycarbonyl | conc. | concentrated |
| Bu | butyl | B.p., b.p. | Boiling point |
| BuLi | n-butyllithium | LC | liquid chromatography |
| c | concentration | Hunig base | N-ethyl-diisopropylamine |
| cHex | cyclohexane | i | iso |
| CDI | carbonyldiimidazole | mCPBA | meta-chloroperbenzoic acid |
| CSI | chlorosulphonyl isocyanate | min | minutes |
| DC, TLC | thin layer chromatography | Me | methyl |
| DCC | dicyclohexylcarbodiimide | MS | mass spectrometry |
| DCM | dichloromethane | NMP | N-methylpyrrolidone |
| DIPEA | ethyldiisopropylamine (Hunig | NMR | nuclear magnetic resonance |
| | base) | | |
| DMAP | N,N-dimethylaminopyridine | Ph | phenyl |
| DMF | N,N-dimethylformamide | Pr | propyl |
| DMA | N,N-dimethylacetamide | rac | racemic |
| DMSO | dimethylsulphoxide | R_{f} (Rf) | Retention factor |
| EE | ethylacetate (ethyl acetate) | RP | Reversed phase |
| ESI | electron spray ionization | RT | ambient temperature or |
| | | | retention time (HPLC) |
| Et | ethyl | t | tertiary |
| h | hour | THF | tetrahydrofuran |
| hex | hexyl | TBTU | O-(benzotriazol-1-yl)- |
| | | | N,N,N′,N′-tetramethyl- |
| | | | uronium tetrafluoroborate |
| HPLC | high performance liquid | UV | ultraviolet |
| | chromatography | | |
| LDA | Lithium diisopropylamide | | |

The Examples that follow illustrate the present invention without restricting its scope.

### General

Unless stated to the contrary, all the reactions are carried out in commercially obtainable apparatus by methods conventionally used in chemical laboratories.

The solvents used are bought in analytical grade and used without further purification. All the reagents are used directly without purification in the synthesis.

Starting materials sensitive to air and/or moisture are stored under argon and corresponding reactions and manipulations using them are carried out under protective gas (nitrogen or argon).

### Chromatography

For preparative medium pressure chromatography (MPLC, normal phase) silica gel made by

Millipore (name: Granula Silica Si-60A 35-70 μm) or C-18 RP-silica gel (RP-phase) made by

Macherey Nagel (name: Polygoprep 100-50 C18) is used.

The thin layer chromatography is carried out on ready-made silica gel 60 TLC plates on glass (with fluorescence indicator F-254) made by Merck.

For the preparative high pressure chromatography (HPLC) columns made by Waters are used (name: XTerra Prep. MS C18, 5 μM, 30*100 mm or XTerra Prep. MS C18, 5 μm, 50*100 mm OBD or Symmetry C18, 5 μm, 19*100 mm), the analytical HPLC (reaction control) is carried out with columns made by Agilent (name: Zorbax SB-C8, 5 μm, 21.2*50 mm).

For the chiral high pressure chromatography (HPLC) columns made by Daicel Chemical Industries, Ltd. are used (name: Chiralpak AD-H or Chiralpak AS or Chiracel OD-RH or Chiracel OD-H or Chiracel OJ-H in various sizes and 5 μm material).

### Nuclear Resonance Spectroscopy (NMR)

The nuclear resonance spectra are taken up in deuterated dimethylsulphoxide-d6 as solvent. If other solvents are used, these are explicitly mentioned in the Examples or in the methods. The chemical shift is specified in relation to the standard tetramethylsilane (δ=0.00 ppm). The measurements are obtained using an Avance 400 (400 MHz-NMR-spectrometer) or an Avance 500 (500 MHz-NMR spectrometer) made by Bruker Biospin GmbH.

### HPLC-Mass Spectroscopy/UV-Spectrometry

The retention times/MS-ESI⁺ for characterising the Examples are generated using an HPLC-MS apparatus (high performance liquid chromatography with mass detector) made by Agilent. The apparatus is constructed so that a diode array detector (G1315B made by Agilent) and a mass detector (1100 LS-MSD SL; G1946D; Agilent) are connected in series downstream of the chromatography apparatus (column: XTerra MS C18, 2.5 μm, 2.1*30 mm, Waters or Synergi POLAR-RP 80A; 4 μm, Phenomenex).

The apparatus is operated with a flow of 1.1 ml/min. For a separation process a gradient is run through within 3.1 min (start of gradient: 95% water and 5% acetonitrile; end of gradient: 5% water and 95% acetonitrile; in each case 0.1% formic acid is added to the two solvents).

### Melting Points

Melting points were obtained using a type B-540 apparatus made by Büchi and have not been corrected.

Where the preparation of the starting compounds is not described, they are commercially available or may be prepared analogously to known compounds or processes described herein.

### Preparation of the Compounds According to the Invention

The compounds according to the invention may be prepared by the methods of synthesis described hereinafter, with the substituents of the general formulae having the meanings given above. These processes are intended to illustrate the invention without restricting its subject matter and the scope of the compounds claimed to the content of these Examples.

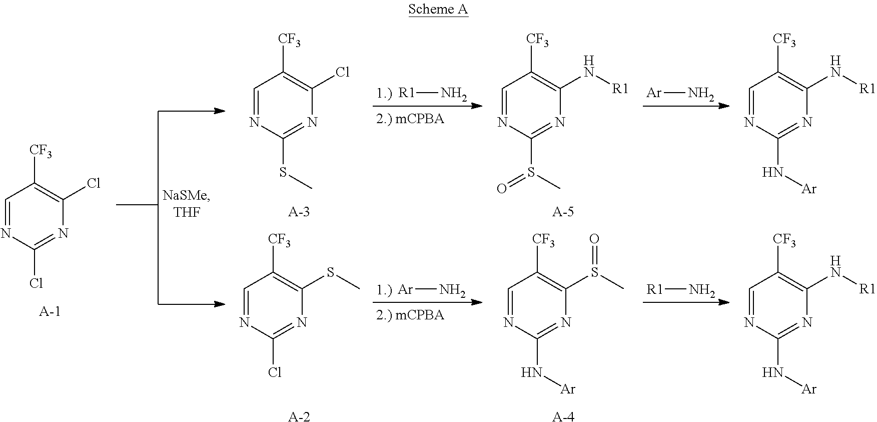

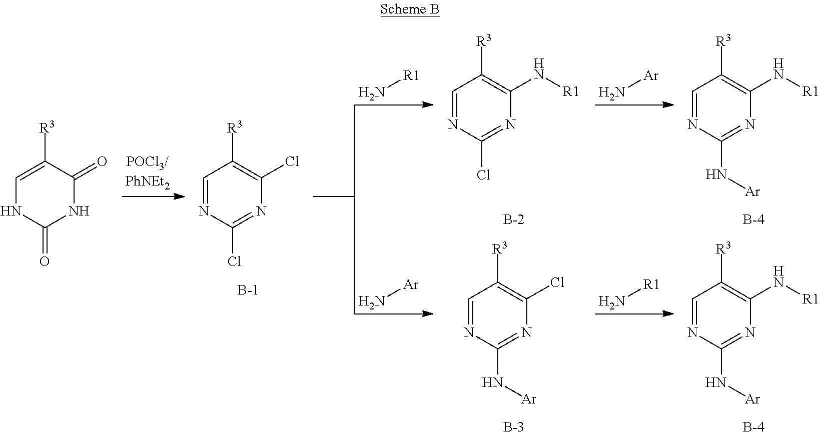

Optionally, after the formation of the diaminopyrimidine, transformation of one or more functional groups is also possible.

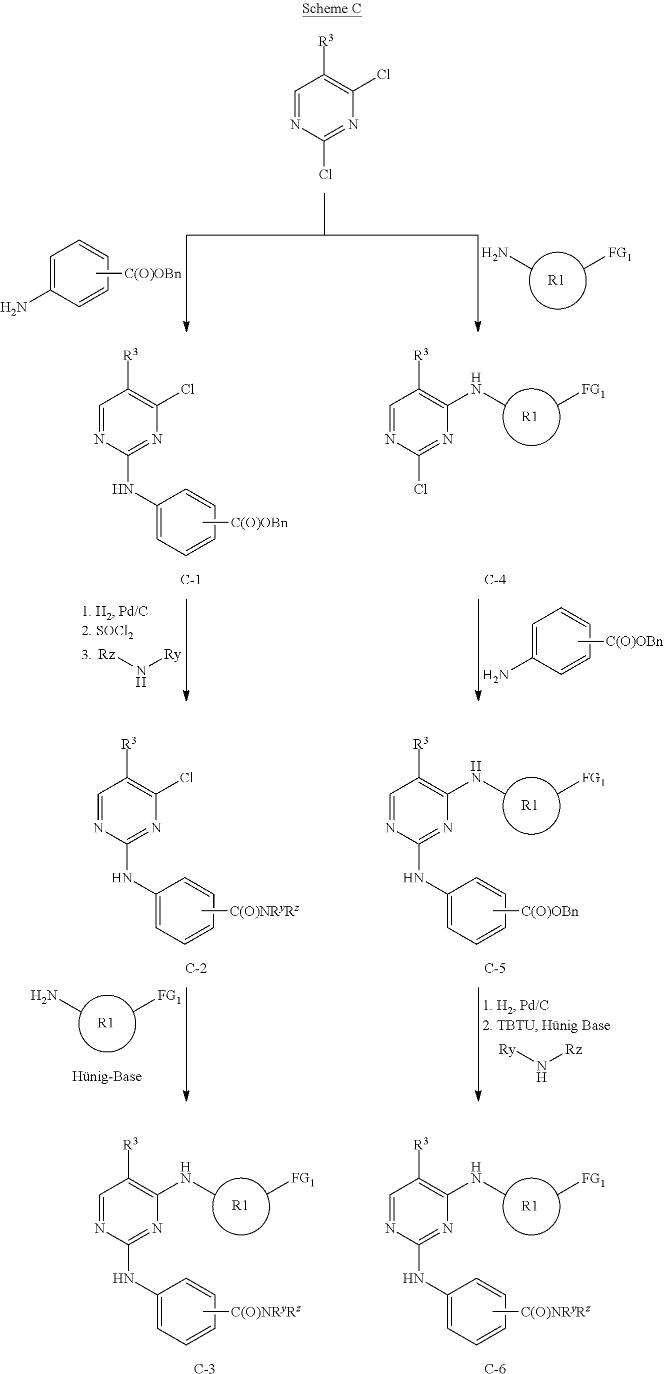

Optionally, after the formation of the diaminopyrimidine, transformation of one or more functional groups (FG) is possible. This is described in the Examples, where relevant.

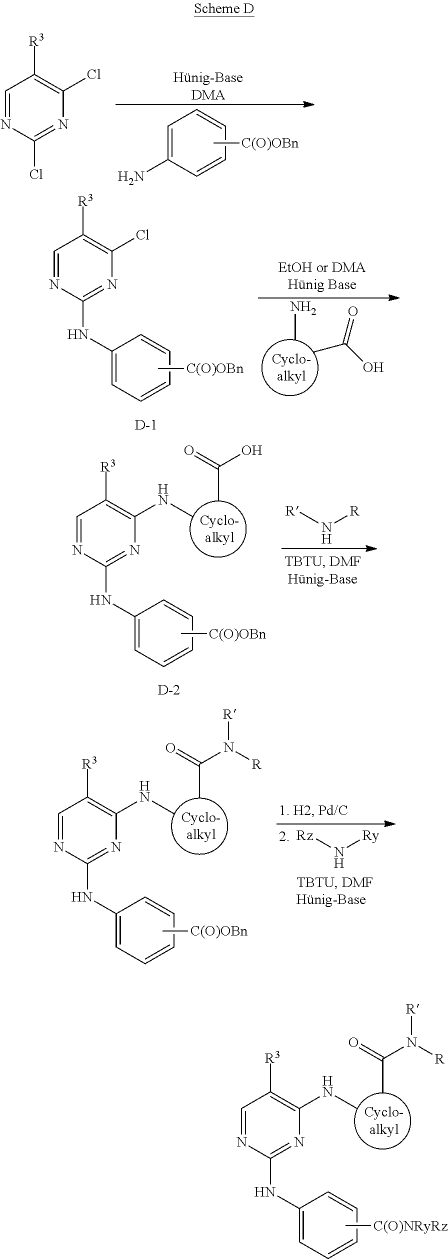

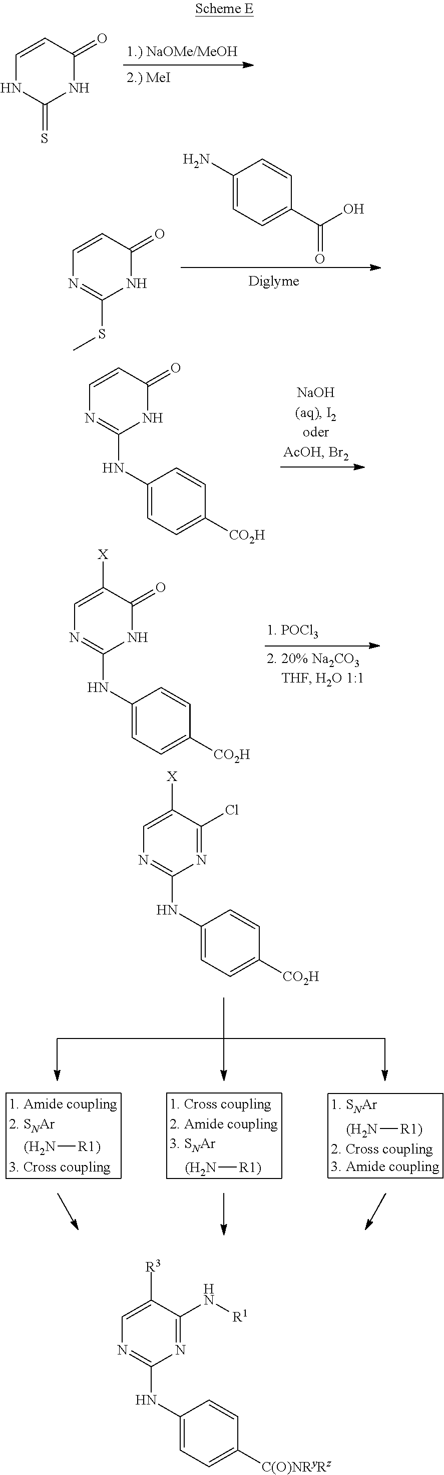

### Preparation of Starting Compounds

Unless otherwise stated all the starting materials are bought from commercial suppliers and used directly in the syntheses. Substances described in the literature are prepared according to the published methods of synthesis.

### A-1) 2,4-dichloro-5-trifluoromethyl-pyrimidine

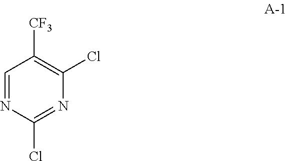 [SMILES: FC(F)(F)c1cnc(Cl)nc1Cl]

48 g (267 mmol) 5-trifluoromethyluracil is suspended in 210 mL phosphorus oxychloride (POCl₃) while moisture is excluded. 47.7 g (320 mmol, 1.2 eq) diethylaniline is slowly added dropwise to this suspension, such that the temperature remains between 25° C. and 30° C. After the addition has ended the mixture is stirred for another 5-10 min in the water bath and the mixture is heated for 5-6 h at 80-90° C. while moisture is excluded. The excess POCl₃ is destroyed by stirring into about 1200 g sulphuric acid-containing ice water and the aqueous phase is immediately extracted 3× with in each case 500 ml ether or t-butyl-methyl-ether. The combined ethereal extracts are washed 2× with 300 mL sulphuric acid-containing ice water (about 0.1 M) and with cold saline solution and immediately dried on sodium sulphate. The drying agent is filtered off and the solvent is eliminated in vacuo. The residue is distilled in vacuo (10 mbar) through a short column (20 cm) (head temperature: 65-70° C.), to obtain 35.3 g (0.163 mol, 61%) of a colourless liquid which is poured off and stored under argon. DC: R_{f}=0.83 (cHex:EE=3:1)

### A-2) 2-chloro-4-methylsulphanyl-5-trifluoromethyl-pyrimidine and

### A-3) 4-chloro-2-methylsulphanyl-5-trifluoromethyl-pyrimidine

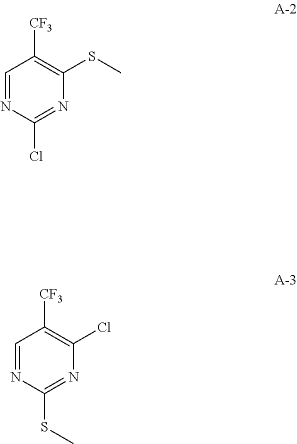

5 g (23 mmol) 2,4-dichloro-5-trifluoromethyl-pyrimidine is dissolved in 40 mL THF, the solution is adjusted to −25° C. and 1.8 g (25.3 mmol, 1.1 eq) sodium thiomethoxide is added. The mixture is stirred for 1 h at −25° C. and then without cooling stirred overnight at RT. Then it is diluted with dichloromethane and washed 3× with 1 N HCl. The organic phase is dried on magnesium sulphate and evaporated down in vacuo. The crude product is purified by column chromatography (silica gel, cyclohexane/dichloromethane; from 90/10 to 80/20% in about 20 min). 1.56 g (6.8 mmol, 30%) of the product A-3 and 1.46 g (6.4 mmol, 28%) of the product A-2 are isolated as colourless oils. In addition 0.24 g (4%) of 2,4-bis-methylsulphanyl-5-trifluoromethyl-pyrimidine may be isolated as a colourless solid.

| | product A-3 | product A-2 |
|---|---|---|
| R_{f} (cHex:CH₂Cl₂ 1:1) | 0.48 | 0.40 |

The structural analysis is carried out by chemical derivatisation and subsequent NMR spectroscopy. For this, A-2 and A-3 are first of all dehalogenated separately in THF at 100° C., 5 bar H₂, Pd/C and Pd(OH)₂ in a ratio of 1:1 in each case. Thanks to the different symmetry characteristics of the products formed it is possible to identify the regioisomers clearly.

### 4-amino-N-methyl-N-phenyl-benzenesulphonamide (Educt in Example 1)

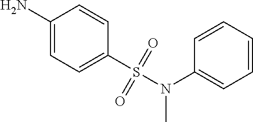 [SMILES: CN(c1ccccc1)S(=O)(=O)c1ccc(N)cc1]

9.5 ml (85.7 mmol, 98%) N-methylaniline is dissolved in 100 mL dichloromethane and at 0° C. 20 g (85.7 mmol, 95%) 4-nitrobenzolsulphonyl chloride, dissolved in 150 mL dichloromethane, is added dropwise and the mixture is stirred for another 1.5 h. The organic phase is washed with saturated, aqueous sodium carbonate solution and dried on sodium sulphate. Finally it is filtered through silica gel and once all the volatile constituents have been eliminated in vacuo 24.6 g of crude N-methyl-4-nitro-N-phenyl-benzenesulphonamide are obtained.

14.6 g (49.9 mmol) of the nitrosulphonic acid amide is dissolved in 100 mL THF/MeOH 1/1. After addition of Pd/C (10%) the mixture is stirred for 16 h at 50° C. unter 5 bar H₂ pressure. After the addition of molecular sieve to bind water, the further addition of Pd/C and more stifling under hydrogenation conditions (5 bar H₂ pressure, 60° C.) for 16 h, 13.1 g (48.9 mmol, 100%) of crude A-4a is obtained as a beige solid. This crude product is used in the synthesis without any further purification.

4-amino-N-phenyl-benzenesulphonamide and 4-amino-N,N-dimethyl-benzenesulphonamide are prepared analogously (educts in Example 2 and 3). The method described is a generally applicable process for preparing substituted or unsubstituted aminobenzenesulphonic acid amides from the corresponding nitrobenzenesulphonic acid chlorides.

### General Procedure Laid Down for the Synthesis of Compounds of Type B-2

A correspondingly R3-substituted 2,4-dichloropyrimidine B-1 (commercially obtainable or prepared by chlorinating the corresponding uracil as described by way of example for A-1) is dissolved in THF (or dioxane, DMA, NMP, acetone) (about 2-5 mL pro mmol), 1-1.6 eq Hünig base (or triethylamine, potassium carbonate or another suitable base) are added and the temperature of the reaction mixture is adjusted (−78° C. for very reactive pyrimidines, RT or elevated temperature for rather unreactive pyrimidines). Then about 0.75-1 eq of the amine, dissolved in the corresponding solvent (see above), are added and the reaction mixture is stirred for a specified time at the corresponding temperature or thawed or heated for a specified time, depending on the reactivity of the pyrimidine used. After the reaction has ended (reaction monitored by HPLC or DC) the reaction mixture is combined with silica gel and all the volatile constituents are eliminated in vacuo. Purification by column chromatography yields the desired substitution products. Depending on group R3 of the pyrimidine, the two possible regioisomers are obtained in different proportions. They can usually be separated by chromatography.

### B-2a) (±)-(1S*,2R*)-2-(2-chloro-5-trifluoromethyl-pyrimidin-4-ylamino)-cyclopentane-carboxamide

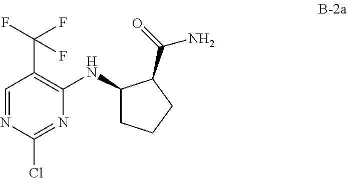 [SMILES: NC(=O)[C@H]1CCC[C@H]1Nc1nc(Cl)ncc1C(F)(F)F]

500 mg (2.3 mmol) A-1 and 636 mg (4.6 mmol, 2 eq) potassium carbonate is suspended in 11 mL acetone, cooled to −70° C., then cis-(±)-(1S,2R)-2-amino-cyclopentanecarboxamide is added. The reaction is left to thaw overnight with stirring at RT and then stirred for another 24 hours at ambient temperature. 40 mL silica gel is then added and all the volatile constituents are eliminated in vacuo. The two regioisomeric products are separated by column chromatography, while the desired regioisomer is the product eluted first (silica gel, cHex/EE 40/60). 218 mg (0.71 mmol, 31%) B-2a and 297 mg (0.96 mmol, 42%) of the regioisomeric product B-2′a are isolated.

R_{f} (B-2a)=0.51 (silica gel, EE), [R_{f} (B-2a′)=0.34]

MS-ESI+: 309 (M+H)⁺

The structure of the two regioisomers is clarified and classified by separate dehalogenation under reductive conditions and subsequent 1H-NMR-spectroscopy of the products (analogously to A-2 and A-3).

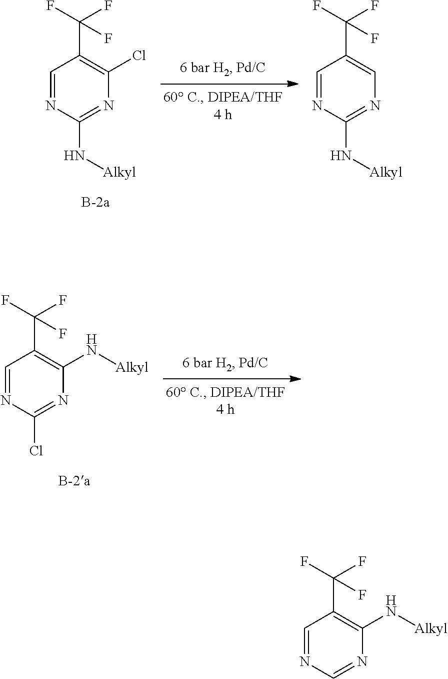

The following Examples of compounds of type B-2 are synthesised analogously.

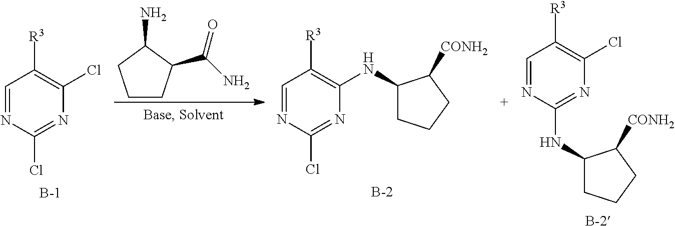

| # | R³ | conditions | B-2:B-2′ | Yield B-2 | R_{f} (B-2) | R_{f} (B-2′) | eluant |
|---|---|---|---|---|---|---|---|
| B-2a | CF₃ | acetone, K₂CO₃, | 42:58 | 31% | 0.51 | 0.34 | EE |
| | | −70° C.-RT, 16 h | | | | | |
| B-2b | Me | DMA, Hünig base, | >85:15 | 83% | 0.25 | not deter- | EE |
| | | 40° C., 24 h | | | | mined | |
| B-2c | NO₂ | acetone, K₂CO₃ | >99:1 | 82% | 0.54 | — | EE |
| | | −70° C., 16 h | | | | | |
| B-2d | F | dichloromethane, | >99:1 | 82% | 0.43 | — | EE |
| | | Hünig base, 0° C.- | | | | | |
| | | RT, 2 days | | | | | |
| B-2e | Cl | dichloromethane, | not deter- | 60% | 0.45 | not deter- | EE |
| | | Hünig base, 0° C.- | mined | | | mined | |
| | | RT, 1 day | | | | | |
| B-2f | i-Pr | DMA, Hünig base, | not deter- | 60% | 0.40 | 0.28 | EE |
| | | 70° C., 24 h | mined | | | | |

The compounds B-2a to B-2f may be reacted with anilines, with acid catalysis, to form compounds of type B-4.

### General Procedure Laid Down for the Synthesis of Compounds of Type B-4

The educt B-2 is dissolved in 1-butanol (or dioxane, DMA, NMP) (about 0.5-4 mL per mmol), 0.1-1 eq HCl in dioxane is added and 1 eq of the aniline and the reaction mixture is refluxed. After the reaction has ended the reaction mixture is combined with silica gel and all the volatile constituents are eliminated in vacuo. Then the mixture is purified by column chromatography. Often, the products are precipitated from the reaction solution even after the end of the reaction and can be directly suction filtered and washed with 1-butanol.

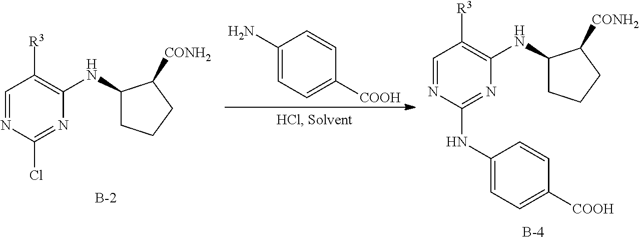

| # | R³ | conditions | Yield B-4 | R_{f} | eluant |
|---|---|---|---|---|---|
| B-4a | CF₃ | is prepared according to | — | 0.37 | DCM:MeOH:AcOH |
| | | Scheme C from C-1 (C- | | | 9:1:0.1 |
| | | 3a≡B-4a) | | | |
| B-4b | Me | 1-butanol, 0.1 eq HCl, | 95% | 0.11 | DCM:MeOH:AcOH |
| | | refluxed for 3 hours | | | 9:1:0.1 |
| B-4c | NO₂ | 1-butanol, 0.1 eq HCl, | 66% | not | — |
| | | refluxed for 4 hours | | deter- | |
| | | | | mined | |
| B-4d | F | 1-butanol, 0.1 eq HCl, | 83% | 0.27 | DCM:MeOH:AcOH |
| | | refluxed for 4 hours | | | 9:1:0.1 |
| B-4e | Cl | 1-butanol, 0.1 eq HCl, | 92% | 0.31 | DCM:MeOH:AcOH |
| | | refluxed for 2 hours | | | 9:1:0.1 |
| B-4f | i-Pr | 1-butanol, 0.1 eq HCl, | 99% | 0.08 | DCM:MeOH:AcOH |
| | | refluxed for 4 hours | | | 9:1:0.1 |

### (4-amino-2-chloro-phenyl)-(4-methyl-piperazin-1-yl)-methanone (Educt in Example 70)

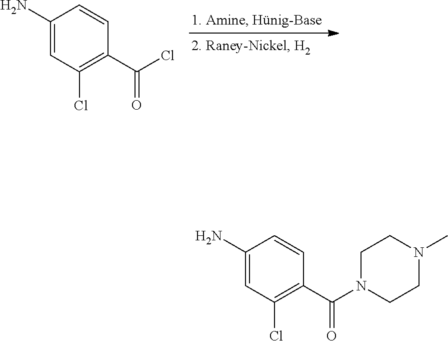 [SMILES: Nc1ccc(C(=O)Cl)c(Cl)c1 ; CN1CCN(C(=O)c2ccc(N)cc2Cl)CC1]

1 ml (8.84 mmol, 1.3 eq) N-methylpiperazine is dissolved in 40 mL dichloromethane and this solution is combined with 1.5 mL (8.84 mmol, 1.3 eq) Hünig base. Then 1-5 g (6.82 mol, 1 eq) nitro-2-chlorbenzoyl chloride, dissolved in 10 mL dichloromethane, is slowly added dropwise while being cooled. After 2 h, 9 mL saturated, aqueous sodium hydrogen carbonate solution is slowly added dropwise with stirring, the organic phase is separated off and the solvent is eliminated in vacuo. The product is purified by column chromatography (silica gel, DCM/MeOH/NH₃ Sep. 1, 2001) and 1.83 g (6.45 mmol, 95%) of the nitrobenzoic acid amide is obtained. The latter is dissolved in 21 THF, 300 mg Raney nickel are added and the mixture is stirred for 16 h at 3 bar H₂ pressure and at RT. After the Raney nickel has been filtered off and the volatile constituents eliminated in vacuo, 1.2 g (4.73 mmol, 73%) (4-amino-2-chloro-phenyl)-(4-methyl-piperazin-1-yl)-methanone is obtained.

R_{f}=0.38 (silica gel, DCM:MeOH:NH₃=9:1:0.1)

MS-ESI⁺: 254 (M+H)⁺

The method is analogously suitable for the synthesis of substituted and unsubstituted aminobenzoic acid amides as used, for example, in the synthesis of Examples 71-75. These Examples are prepared analogously to Example 70. In the synthesis of Examples 106, 107 and 144 m-aminobenzoic acid amides are used which are prepared by the same method.

### cis-(±)-2-amino-cyclopentanecarboxylic acid-isopropylamide

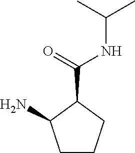

55 mg (0.43 mmol) cis-(±)-2-amino-cyclopentanecarboxylic acid is suspended in 900 μL (25 eq) isopropylamine, and 205 mg (0.064 mmol, 1.5 eq) TBTU and 550 μL DMF are added to this suspension. It is stirred for 16 h and the reaction mixture is taken up in DCM:MeOH:NH₃ 9:1:0.1 and combined with 7 mL silica gel. After all the volatile constituents have been eliminated in vacuo the mixture is chromatographed (silica gel DCM:MeOH:NH₃ 9:1:0.1). 63 mg (0.37 mmol, 86%) colourless solid are obtained.

R_{f}=0.33 (silica gel, DCM:MeOH:NH₃ 85:15:1.5)

### B-2g) (±)-(1S*,2R*)-2-(2-chloro-5-trifluoromethyl-pyrimidin-4-ylamino)-cyclopentane-carboxylic acid isopropylamide

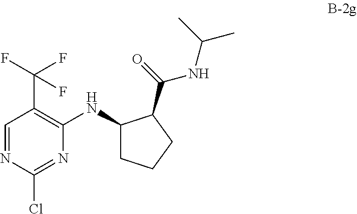 [SMILES: CC(C)NC(=O)[C@H]1CCC[C@H]1Nc1nc(Cl)ncc1C(F)(F)F]

2 g (9.2 mmol) A-1 and 1.8 ml (11.2 mmol, 1.2 eq) Hünig base are dissolved in 60 mL THF, the mixture is cooled to −78° C., then cis-(±)-2-amino-cyclopentanecarboxylic acid isopropylamide, dissolved in 60 mL THF, is slowly added dropwise at −78° C. The reaction is left to thaw to RT overnight with stifling. Then 40 mL silica gel are added and all the volatile constituents are eliminated in vacuo. The two regioisomeric products are separated by column chromatography, while the desired regioisomer is the product that elutes first (silica gel, cHex/EE from 85/15 to 80/20 within 30 min). 590 mg (1.68 mmol, 24%) B-2g and 690 mg (1.97 mmol, 28%) of the regioisomeric product B-2g′ are isolated.

R_{f} (B-2g)=0.21 (silica gel, cHex:EE 3:1), [R_{f} (B-2g′)=0.10]

MS-ESI+: 351 (M+H)⁺

UVₘₐₓ=246 nm

### 3-fluoro-4-(4-methyl-[1.4]diazepan-1-yl)-phenylamine

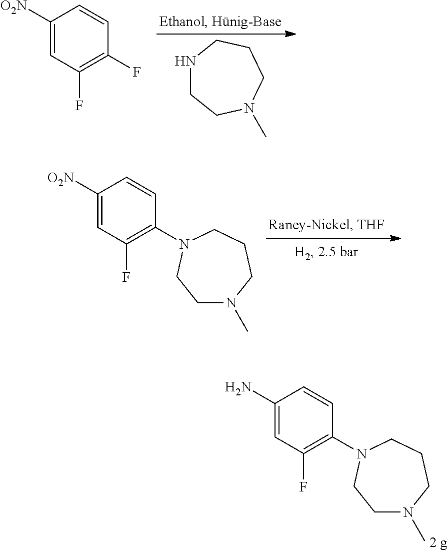

(12.6 mmol) 3,4-difluoronitrobenzene is dissolved in 1.6 ml of ethanol, 2.4 mL (15.1 mmol, 1.2 eq) Hünig base is added and then 1.44 g (12.6 mmol, 1 eq) hexahydro-1-methyl-1H-1.4-diazepine is added dropwise while cooling with ice. After about 12 h stifling at RT the reaction is complete. Then methanol and 50 mL silica gel are added, the volatile constituents are eliminated in vacuo and the mixture is purified by column chromatography (DCM/MeOH 97/3 to 85/15 in 35 min). 3 g (11.9 mmol, 94%) of the nitro compound is obtained.

R_{f}=0.39 (silica gel, DCM:MeOH:NH₃ 9:1:0.1)

MS-ESI⁺: 253 (M+H)⁺

The nitro compound is dissolved in 600 mL THF and combined with about 300 mg Raney nickel. The mixture is hydrogenated for 3 h at an H₂ pressure of 3 bar. The Raney nickel is filtered off and the solution is freed from all volatile constituents in vacuo. 2.15 g (9.6 mmol, 81%) 3-fluoro-4-(4-methyl-[1.4]diazepan-1-yl)-phenylamine is obtained.

R_{f}=0.48 (silica gel, DCM:MeOH:NH₃ 4:1:0.1)

MS-ESI⁺: 224 (M+H)⁺

The anilines which are used as educts in Examples 142-143 are prepared analogously.

### benzyl 4-amino-benzoate

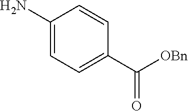 [SMILES: Nc1ccc(C(=O)OCc2ccccc2)cc1]

10.01 g 4-nitrobenzoic acid is suspended in 500 mL acetonitrile and then combined with 15.03 g (108.7 mmol, 1.2 eq) potassium carbonate. 15.40 g (171.0 mmol, 1 eq) benzylbromide ais added dropwise with stirring and the reaction mixture is then heated to 60° C. for 5 h with stirring. It is combined with 750 ml distilled water, extracted 4× with 250 mL EE and, after the organic phases have been combined, dried on sodium sulphate. After the elimination of all the volatile constituents in vacuo the crude product is successively suspended 2× in toluene and all the volatile constituents are eliminated in vacuo (removal of excess benzylbromide). 20.60 g (80.1 mmol) benzyl 4-nitro-benzoate is obtained as a colourless solid, which is used in the next step without further purification.

20.6 g of the benzyl 4-nitro-benzoate are dissolved in 350 mL dioxane and this solution is combined with 6.9 g (49.9 mmol, 0.61 eq) Raney nickel. The mixture is hydrogenated for 16 h with stifling at 5 bar H₂ pressure. The catalyst is filtered off, all the volatile constituents are eliminated in vacuo. 17.0 g (74.8 mmol, 93%) benzyl 4-aminobenzoate is obtained in the form of a colourless solid.

### C-1a) benzyl 4-(4-chloro-5-trifluoromethyl-pyrimidin-2-ylamino)-benzoate

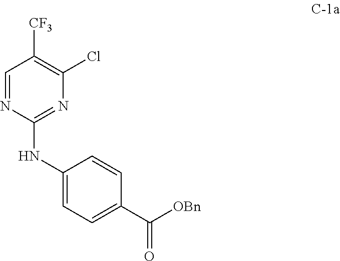 [SMILES: O=C(OCc1ccccc1)c1ccc(Nc2ncc(C(F)(F)F)c(Cl)n2)cc1]

10 g (44 mmol) benzyl 4-aminobenzoate is dissolved in 200 mL DMA, 8 mL Hünig base (0.97 eq) is added and 10.4 g (48.21 mmol) 2,4-dichloro-5-trifluoromethylpyrimidine, dissolved in 50 mL DMA, are added dropwise at RT to the clear solution. The reaction solution is stirred overnight at 60° C., then combined with 300 mL dichloromethane and extracted with distilled water (3×300 mL). The organic phase is dried on sodium sulphate and the solvent is eliminated in vacuo. The crude product is combined with 100 mL MeOH, digested and left to stand for 2 h. Then the mixture is stirred for 10 min, the precipitate is filtered off and washed with methanol (methanolic filtrate contains the unwanted regioisomer of the nucleophilic substitution). Finally the crude product is once more suspended in methanol, filtered off, washed with a little methanol and dried at 60° C. in the vacuum dryer. 8.5 g (20.7 mmol, 43%) of C-1a is obtained in the form of a light yellow solid.

R_{f}=0.71 (silica gel, cHex:EE 1:2)

MS-ESI⁺: 408 (M+H)⁺

### C-2a) [4-(4-chloro-5-trifluoromethyl-pyrimidin-2-ylamino)-phenyl]-(4-methyl-piperazin-1-yl)-methanone

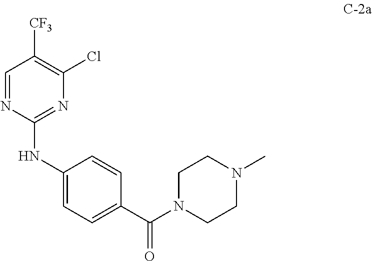 [SMILES: CN1CCN(C(=O)c2ccc(Nc3ncc(C(F)(F)F)c(Cl)n3)cc2)CC1]

2.74 g (6.71 mmol) C-1a is dissolved in 120 mL dioxane, 300 mg palladium hydroxide (20% w/w Pd, 2.14 mmol, 0.32 eq) is added and the mixture is stirred for 16 h at 3 bar H₂ pressure and RT. The reaction mixture is filtered through Celite, the solvent is eliminated in vacuo and 1.87 g (5.89 mmol, 88%) 4-(4-chloro-5-trifluoromethyl-pyrimidin-2-ylamino)-benzoic acid is obtained as a colourless solid, which is used without further purification. 1.1 g (3.46 mmol) of the benzoic acid is combined with 20 mL toluene and 301 μL (4.16 mmol, 1.2 eq) thionyl chloride and refluxed for 1.5 h. All the volatile constituents are eliminated in vacuo and the crude benzoic acid chloride is further reacted directly.

536 mg (1.6 mmol) thereof are dissolved in 4 mL THF and combined with 410 μL (1.5 eq) Hünig base. After the addition of 179 μL (1 eq) N-methylpiperazine the solution is stirred for 16 h at RT. The reaction mixture is poured into about 40 mL distilled water, stirred for 30 min and the aqueous phase is extracted 3× with 50 ml of ethyl acetate. After drying the organic phase on magnesium sulphate, filtration and elimination of the volatile constituents in vacuo 645 mg (1.5 mmol, 94%) C-2a is obtained as a solid.

R_{f}=0.69 (silica gel, CH₂Cl₂:MeOH:NH₃ 5:1:0.1)

MS-ESI+: 400 (M+H)⁺

### C-2b) 4-(4-chloro-5-trifluoromethyl-pyrimidin-2-ylamino)-N-methyl-N-(1-methyl-piperidin-4-yl)-benzamide

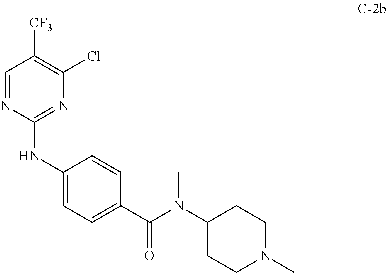 [SMILES: CN1CCC(N(C)C(=O)c2ccc(Nc3ncc(C(F)(F)F)c(Cl)n3)cc2)CC1]

R_{f}=0.30 (silica gel, CH₂Cl₂:MeOH:NH₃ 5:1:0.1)

MS-ESI+: 428 (M+H)⁺

C-2b is prepared analogously to C-2a using methyl-(1-methyl-piperidin-4-yl)-amine.

### benzyl (±)-((1S*,2R*)-2-amino-cyclohexyl)-carbamate

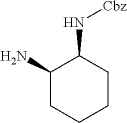 [SMILES: N[C@@H]1CCCC[C@@H]1NC(=O)OCc1ccccc1]

2 mL (16.2 mmol) cis-1,2-diaminocyclohexane and 2.42 g (19.4 mmol, 1.2 eq) 9-borabicyclo[3.3.1]nonane (9-BBN) are dissolved in 8 mL THF/NMP 1/1 and stirred for 45 min at RT. 2.4 mL (16.2 mmol, 1 eq) benzylchloroformate (Cbz-chloride) is added to the slightly cloudy solution. After about 1 h the reaction mixture is combined with distilled water and stirred for a few minutes. Then the aqueous solution is combined with ethylacetate and the aqueous phase is washed 3× with about 50 mL ethylacetate. The product is entirely present in the aqueous phase, contaminants in the organic phase. The aqueous phase is made alkaline with NaHCO₃ (pH 8), mixed with dichloromethane, extracted 3× with 10 mL dichloromethane, the combined organic phases are dried on magnesium sulphate and the solvent is eliminated in vacuo. 2.29 g (9.22 mmol, 57%) benzyl (±)-((1S*,2R*)-2-amino-cyclohexyl)-carbamate is obtained as a colourless oily liquid.

R_{f}=0.45 (silica gel, CH₂Cl₂:MeOH:NH₃ 9:1:0.1)

MS-ESI⁺: 249 (M+H)⁺

### C-3a) benzyl (±)-((1S*,2R*)-2-{2-[4-(4-methyl-piperazin-1-carbonyl)-phenylamino]-5-trifluoromethyl-pyrimidin-4-ylamino}-cyclohexyl)-carbamate

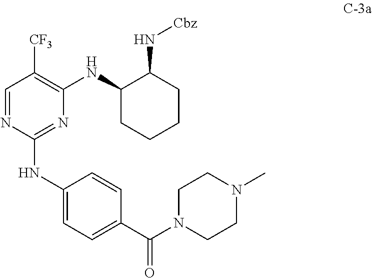 [SMILES: CN1CCN(C(=O)c2ccc(Nc3ncc(C(F)(F)F)c(N[C@@H]4CCCC[C@@H]4NC(=O)OCc4ccccc4)n3)cc2)CC1]

800 mg (2 mmol) C-2a is dissolved with in 1 mL NMP, 569 mg (2.4 mmol, 1.2 eq) benzyl (±)-((1S*,2R*)-2-amino-cyclohexyl)-carbamate and then 521 μL (3 mmol, 1.5 eq) Hünig base are added. After 48 h at 70° C. the reaction has stopped. After elimination of the solvent in vacuo the crude product is purified by column chromatography (DCM/MeOH/NH₃ from 19/1/0.1 to 9/1/0.1) and 826 mg (1.35 mmol, 68%) of the product is obtained in the form of a colourless resin.

MS-ESI⁺: 612 (M+H)⁺

### C-3b) (±)-{4-[4-((1R*,2S*)-2-amino-cyclohexylamino)-5-trifluoromethyl-pyrimidin-2-ylamino]-phenyl}-(4-methyl-piperazin-1-yl)-methanone

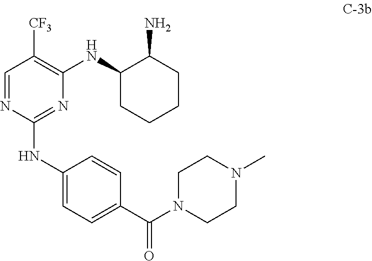 [SMILES: CN1CCN(C(=O)c2ccc(Nc3ncc(C(F)(F)F)c(N[C@@H]4CCCC[C@@H]4N)n3)cc2)CC1]

112 mg (0.18 mmol) C-3a is dissolved in DMF (10 mL) and combined with distilled water (1 mL). Then another 9 mL of DMF is added, the solution is transferred into a hydration apparatus and combined with Pd/C (200 mg, 5% Pd). The reaction solution is stirred for 12 h at an H₂ pressure of 4 bar. The reaction mixture is taken up in dichloromethane and combined with 10 mL RP-gel and all the volatile constituents are eliminated in vacuo. The purification is done by column chromatography (RP-phase, acetonitrile/water from 5/95 to 95/5 in 20 min). After combining the product fractions and freeze-drying, 27 mg (0.06 mmol, 30%) of the desired product is obtained as a colourless solid.

MS-ESI⁺: 478 (M+H)⁺

### C-3c) (±)-(1S*,2R*)-2-{2-[4-(4-methyl-piperazin-1-carbonyl)-phenylamino]-5-trifluoromethyl-pyrimidin-4-ylamino}-cycloheptanecarboxylic acid

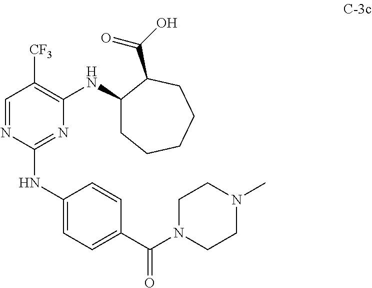 [SMILES: CN1CCN(C(=O)c2ccc(Nc3ncc(C(F)(F)F)c(N[C@@H]4CCCCC[C@@H]4C(=O)O)n3)cc2)CC1]

440 mg (1.1 mmol) C-2a is dissolved in 500 μL NMP and combined with 565 μL Hünig base (3.3 mmol, 3 eq) and 256 mg cis-2-aminocycloheptanecarboxylic acid (racemic). The reaction mixture is placed in an oil bath maintained at 100° C. and is heated to this temperature for 8 h with stifling. After the end of the reaction the reaction mixture is taken up in methanol, combined with 20 mL RP-gel and all the volatile constituents are eliminated in vacuo. Purification is carried out by phase reversal (eluant: acetonitrile/water (15/85 to 35/65 in 15 min). After combining the product fractions and freeze-drying, 160 mg (0.31 mmol, 28%) of the desired product is obtained as a colourless solid.

MS-ESI⁺: 521 (M+H)⁺

### C-3d) (±)-(1S*,2R*)-2-{2-[4-(4-methyl-piperazin-1-carbonyl)-phenylamino]-5-trifluoromethyl-pyrimidin-4-ylamino}-cyclopentanecarboxylic acid

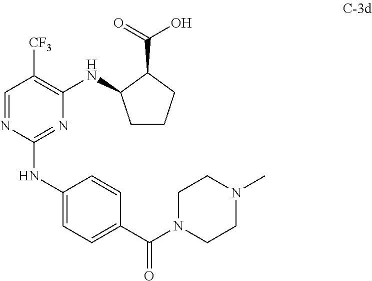 [SMILES: CN1CCN(C(=O)c2ccc(Nc3ncc(C(F)(F)F)c(N[C@@H]4CCC[C@@H]4C(=O)O)n3)cc2)CC1]

563 mg (1.13 mmol) C-2a is dissolved in 5 mL 1-butanol and to this is added 163 mg cis-2-amino-1-cyclopentanecarboxylic acid (racemic). After the addition of 540 μL Hünig base the mixture is heated to 110° C. for about 60 min (microwave, CEM, 100 W). The reaction mixture is evaporated down in vacuo, stirred with about 100 mL water and extracted 3× with 50 mL ethyl acetate. The combined organic phases are dried on magnesium sulphate and the solvent is eliminated in vacuo. 530 mg (1.08 mmol, 96%) C-3d are obtained.

MS-ESI⁺: 493 (M+H)⁺

C-3e is prepared analogously using DMA as solvent and C-2b as starting material.

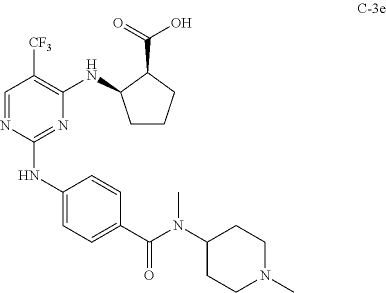 [SMILES: CN1CCC(N(C)C(=O)c2ccc(Nc3ncc(C(F)(F)F)c(N[C@@H]4CCC[C@@H]4C(=O)O)n3)cc2)CC1]

MS-ESI⁺: 521 (M+H)⁺

### C-3f) (1S,3R)-3-(2-{4-[methyl-(1-methyl-piperidin-4-yl)-carbamoyl]-phenylamino}-5-trifluoromethyl-pyrimidin-4-ylamino)-cyclopentanecarboxylic acid

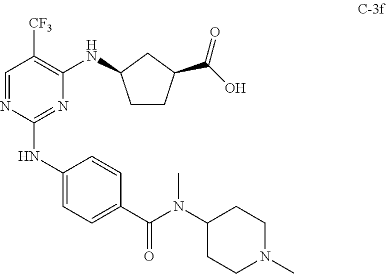 [SMILES: CN1CCC(N(C)C(=O)c2ccc(Nc3ncc(C(F)(F)F)c(N[C@@H]4CC[C@H](C(=O)O)C4)n3)cc2)CC1]

200 mg C-2b is dissolved in 750 μL DMA and 160 μL (0.93 mmol, 2 eq) Hünig base is added. Then 72 mg (0.56 mmol, 1.2 eq) (1S,3R)-3-aminocyclopentane-carboxylic acid is added and the reaction mixture is heated to 120° C. for 40 min. The reaction mixture is combined with RP-gel, the volatile constituents are eliminated in vacuo and the product is purified by column chromatography through an RP-phase and isolated (from 85% water (+0.2% HCOOH) and 15% acetonitrile (+0.2% HCOOH) to 76% water and 24% acetonitrile in 20 min). Corresponding product fractions are combined, freed from the solvent by freeze-drying and 150 mg (0.29 mmol, 62%) C-3f is obtained as a colourless film.

### (±)-trans-2-aminocyclopentanecarboxamide

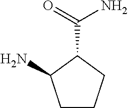

The compound is prepared according to the literature (Csomos et al., 2002).

### D-2a) benzyl 4-[4-((1R,2S)-2-carboxy-cyclopentylamino)-5-trifluoromethyl-pyrimidin-2-ylamino]-benzoate

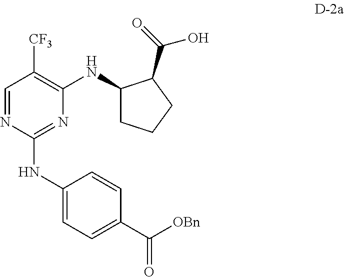 [SMILES: O=C(OCc1ccccc1)c1ccc(Nc2ncc(C(F)(F)F)c(N[C@@H]3CCC[C@@H]3C(=O)O)n2)cc1]

2.05 g (5 mmol) C-1a and 1 g (1S,2R)-(+)-2-amino-1-cyclopentanecarboxylic acid hydrochloride (6 mmol, 1.2 eq) are placed in 18 mL ethanol. 7.3 ml (42.5 mmol, 3.4 eq) Hünig base is added and the mixture is stirred for 4 h at 70° C. The reaction mixture is stirred into 275 mL water, filtered to remove the undissolved matter, the filtrate is adjusted to pH 2 with saturated aqueous KHSO₄ solution, stirred for 5 min and the precipitate formed is suction filtered. The crude product is washed with water, dried in vacuo and 2.37 g (4.74 mmol, 94%) D-2a is obtained in the form of a light beige solid.

MS-ESI⁺: 501 (M+H)⁺

The synthesis with (1R,2S)-(−)-2-amino-1-cyclopentanecarboxylic acid- or (1R*,2S*)-(±)-2-amino-1-cyclopentanecarboxylic acid derivative is carried out analogously. The corresponding products are designated D-2b (chiral, enantiomer of D-2a) and D-2c (rac).

### Preparation of (1S,2R)-2-aminocyclopentanecarboxylic acid hydrochloride

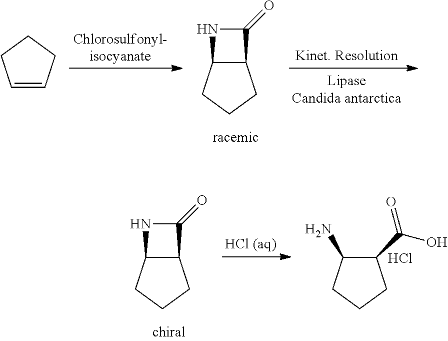 [SMILES: C1=CCCC1 ; O=C1N[C@@H]2CCC[C@H]12 ; O=C1N[C@@H]2CCC[C@H]12 ; N[C@@H]1CCC[C@@H]1C(=O)O ; Cl]

22.64 mL (0.26 mol, 0.95 eq) CSI is added dropwise to 23 mL (0.273 mol, 1 eq) cyclopentene at −75° C. under argon. During the addition the reaction temperature is always kept below −65° C. The reaction is allowed to come up to RT within 2 h and stirred further overnight. The reductive working up is carried out by dropwise addition of the reaction solution to a solution of 600 mL ice/water with 60 g sodium sulphite and 180 g NaHCO₃. The aqueous phase is extracted 4× with 200 mL dichloromethane, the organic phases are combined, dried on magnesium sulphate and all the volatile constituents are eliminated in vacuo. 25.75 g (85%) of slightly yellowish crystals are obtained.

These are dissolved in 400 mL diisopropylether, 1.6 mL water and 20 g resin-bonded lipolase (lipase acrylic resin from candida antartica, Sigma-Aldrich) is added and the mixture is shaken for 11 days at 60° C. The reaction suspension is filtered through Celite, washed with diisopropylether and the filtrate is evaporated to dryness. The yellowish oil obtained is taken up in 200 mL dichloromethane and washed with about 150 mL of saturated NaHCO₃ solution. The aqueous phase is extracted 3× with dichloromethane, the organic phases are combined and dried on magnesium sulphate. After the elimination of all the volatile constituents in vacuo 8.93 g of the chiral lactam is obtained in the form of a yellowish oil.

The latter product is dissolved in 10 mL water and 10 mL 37% HCl (aq) are added while cooling with an ice bath and stifling. After 10 min stifling at 0° C. the reaction solution is left to stand overnight at RT. The crystals precipitated are filtered off, washed with a little acetonitrile and dried under a high vacuum. The mother liquor is evaporated almost to dryness, the crystals precipitated are filtered off, washed with acetonitrile and also dried under a high vacuum. 11.74 g (70.9 mmol, 31% based on the racemic lactam) of colourless crystals of the hydrochloride of (1S,2R)-2-aminocyclopentanecarboxylic acid are obtained.

(The enantiomeric acid has precipitated during the step of kinetic resolution and is contained in the precipitate which was separated off by filtration through Celite).

The synthesis sequence is described in the literature (Forro and Fueloep, 2003).

### D-3a) benzyl 4-[4-((1R,2S)-2-isopropylcarbamoyl-cyclopentylamino)-5-trifluoromethyl-pyrimidin-2-ylamino]-benzoate

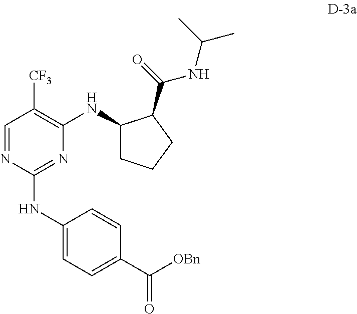 [SMILES: CC(C)NC(=O)[C@H]1CCC[C@H]1Nc1nc(Nc2ccc(C(=O)OCc3ccccc3)cc2)ncc1C(F)(F)F]

2.59 g (4.9 mmol) D-2a, 2.21 g (6.9 mmol, 1.4 eq) TBTU and 4.21 mL (24.6 mmol, 5 eq) Hünig base are dissolved in 75 mL DMF and stirred for 20 min at RT. Then 0.63 ml (7.38 mmol, 1.5 eq) isopropylamine is added and the mixture is stirred overnight at RT. It is suction filtered through basic aluminium oxide, washed with DMF and the mother liquor is stirred into 400 mL water, stirred for another 30 min and the precipitate is suction filtered. The crude product is washed with water and dried in vacuo. For purification it is stirred with 50 ml acetonitrile for 30 min at 5° C., suction filtered, washed with some cold acetonitrile and the residue is dried in vacuo. 2.13 g (3.9 mmol, 80%) D-3a are obtained in the form of a light beige solid.

R_{f}=0.53 (silica gel, cHx:EE 1:1)

MS-ESI⁺: 542 (M+H)⁺

### D-4a) 4-[4-((1R,2S)-2-isopropylcarbamoyl-cyclopentylamino)-5-trifluoromethyl-pyrimidin-2-ylamino]-benzoic acid

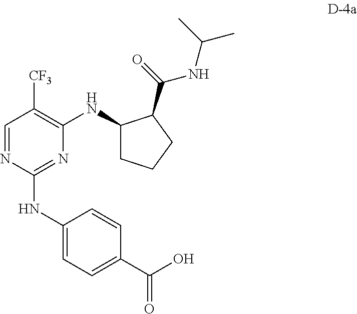 [SMILES: CC(C)NC(=O)[C@H]1CCC[C@H]1Nc1nc(Nc2ccc(C(=O)O)cc2)ncc1C(F)(F)F]

2.13 g (3.9 mmol) D-3a is dissolved in 150 mL THF and 250 mg palladium hydroxide/C-catalyst (20 wt. % Pd on charcoal) are added. The mixture is hydrogenated for 16 h at an H₂ pressure of 6 bar with stifling at RT. Then 30 mL methanol is added, the catalyst is filtered through kieselguhr, washed with methanol and the filtrate is evaporated down. The residue is boiled with 45 mL ethanol, slowly cooled to 5° C., stirred for another 1 h and then suction filtered and washed with cold ethanol. 2.46 g (3.2 mmol, 82%) of the acid D-4a is obtained.

R_{f}=0.46 (silica gel, CH₂Cl₂:MeOH:AcOH 5:1:0.1)

MS-ESI⁺: 452 (M+H)⁺

The enantiomeric compound and racemate are synthesised analogously.

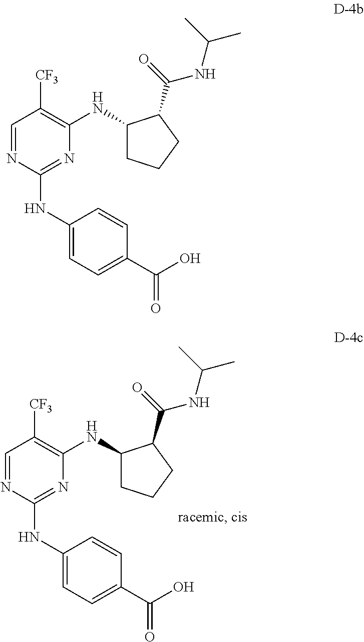

### D-5c) t-butyl (±)-{4-[4-((1R*,2S*)-2-isopropylcarbamoyl-cyclopentylamino)-5-trifluoromethyl-pyrimidin-2-ylamino]-phenyl}-carbamate

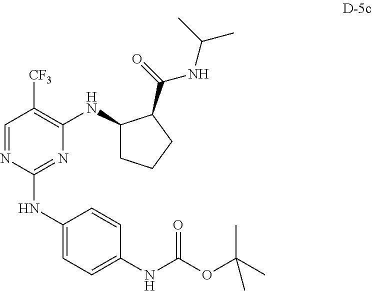 [SMILES: CC(C)NC(=O)[C@H]1CCC[C@H]1Nc1nc(Nc2ccc(NC(=O)OC(C)(C)C)cc2)ncc1C(F)(F)F]

450 mg (1 mmol) D-4c is dissolved in 1.8 mL dry toluene and 222 μL (1.3 mmol, 1.3 eq) Hünig base and 940 μL t-butanol are added successively. Then 258 μL diphenylphosphorylazide are added and the mixture is heated to 80° C. for 16 h. The reaction mixture is combined with 20 mL ethyl acetate, washed 2× with 20 mL of 0.5 M NaOH solution and the aqueous phase is counter-washed 2× with 20 ml ethyl acetate. The combined organic phases are washed with saturated, aqueous sodium chloride solution, insoluble constituents are filtered off, the filtrate is dried on magnesium chloride and the solvent is eliminated in vacuo. 461 mg (0.88 mmol, 89%) D-5c is obtained in the form of a yellowish solid.

MS-ESI⁺: 523 (M+H)⁺

### D-6c) (±)-(1S*,2R*)-2-[2-(4-amino-phenylamino)-5-trifluoromethyl-pyrimidin-4-ylamino]-cyclopentanecarboxylic acid-isopropylamide

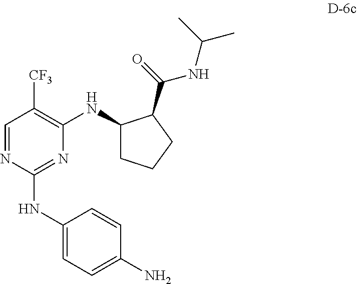 [SMILES: CC(C)NC(=O)[C@H]1CCC[C@H]1Nc1nc(Nc2ccc(N)cc2)ncc1C(F)(F)F]

461 mg (0.88 mmol) D-5c is dissolved in 5 mL dichloromethane, 2 mL trifluoroacetic acid is added and the mixture is stirred for 1 h at RT. The reaction mixture is stirred into 50 mL water and the aqueous phase is washed with 50 mL ethyl acetate. The organic phase is extracted another 2× with 30 mL 10% hydrochloric acid, the aqeuous phases are combined, adjusted to pH 10 with 10% sodium hydroxide solution and extracted 3× with 50 ml ethyl acetate. The combined organic phases are dried on magnesium sulphate, the volatile constituents are eliminated in vacuo and 243 mg (0.58 mmol, 65%) D-6c is obtained as a colourless solid.

R_{f}=0.08 (silica gel, cHex:EE 1:1)

MS-ESI⁺: 423 (M+H)⁺

### E-1) 2-methylsulphanyl-1H-pyrimidin-4-one

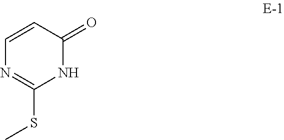

20 g (153 mmol) 2-thiouracil is suspended in 250 mL methanol and then 8.7 g (152.9 mmol, 1 eq) of sodium methoxide is added. The solution is stirred for 5 min at RT and then 12.4 mL (198.8 mmol, 1.3 eq) of methyl iodide is added dropwise. The reaction mixture is stirred overnight, then poured onto water and extracted 3× with about 150 ml chloroform. The combined organic phases are dried on magnesium sulphate, the solvent is eliminated in vacuo and 16 g (121.5 mmol, 74%) E-1 is obtained in the form of a colourless solid.

### E-2) 4-(6-oxo-1,6-dihydro-pyrimidin-2-ylamino)-benzoic acid

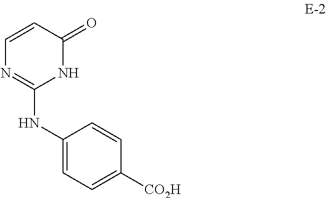 [SMILES: O=C(O)c1ccc(Nc2nccc(=O)[nH]2)cc1]

4.1 g (28.8 mmol) E-1 is dissolved in 10 mL diglyme (diethyleneglycol dimethylether) and this solution is combined with 4.79 g (34.6 mmol, 1.2 eq) 4-aminobenzoic acid. The reaction mixture is refluxed for 16 h. After cooling to RT the precipitate is suction filtered, washed with a little diglyme, then with diethyl ether and dried in vacuo. 5.27 g (22.8 mmol, 79%) E-2 are obtained as a colourless solid.

MS-ESI⁺: 232 (M+H)⁺

### E-3a) 4-(5-iodine-6-oxo-1,6-dihydro-pyrimidin-2-ylamino)-benzoic acid

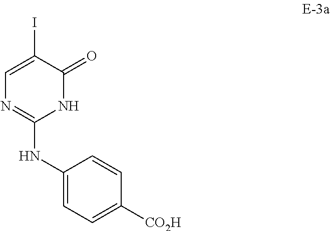 [SMILES: O=C(O)c1ccc(Nc2ncc(I)c(=O)[nH]2)cc1]

9 g (38.9 mmol) E-2 is placed in 100 mL water, 2.18 g NaOH (54.5 mmol, 1.4 eq) is added. The solution is combined with 11.9 g (46.7 mol, 1.2 eq) iodine and stirred for 3 h at 65° C. After cooling to 50° C. sodium thiosulphate pentahydrate is added to eliminate excess iodine, then the mixture is stirred for another 1 h and cooled to RT. The brownish precipitate is suction filtered, washed with water and dried in vacuo. 13.7 g (38.4 mmol, 82%) E-3a is obtained.

MS-ESI⁺: 358 (M+H)⁺

### E-3b) 4-(5-bromo-6-oxo-1,6-dihydro-pyrimidin-2-ylamino)-benzoic acid

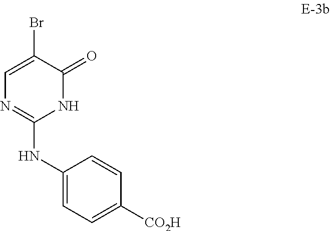 [SMILES: O=C(O)c1ccc(Nc2ncc(Br)c(=O)[nH]2)cc1]

9 g (38.9 mmol) E-2 is placed in 10 mL acetic acid and to this a solution of 2.1 mL (40.9 mmol 1.05 eq) bromine in 50 mL acetic acid is added dropwise and the mixture is stirred for about 1 h at RT. The reaction mixture is stirred into 800 mL water, the precipitate is suction filtered and the brownish precipitate obtained is washed with water and dried in vacuo. 11.5 g (37.1 mmol, 95%) E-3b is obtained as a colourless solid.

R_{f}=0.27 (silica gel, EE:MeOH 7:3)

MS-ESI⁺: 309/311 (M+H)⁺ (1×Br)

### E-4a) 4-(4-chloro-5-iodo-pyrimidin-2-ylamino)-benzoyl chloride and

### E-5a) 4-(4-chloro-5-iodo-pyrimidin-2-ylamino)-benzoic acid

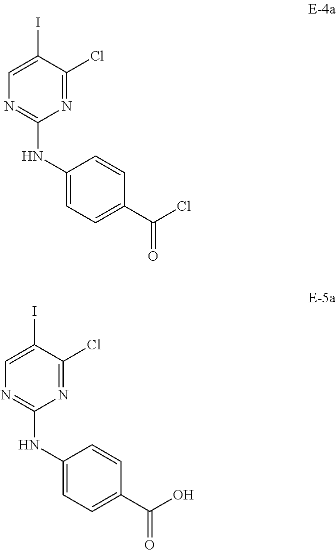 [SMILES: O=C(Cl)c1ccc(Nc2ncc(I)c(Cl)n2)cc1 ; O=C(O)c1ccc(Nc2ncc(I)c(Cl)n2)cc1]

6.5 g (18.2 mmol) E-3a is suspended in 80 mL phosphorus oxychloride and the mixture is refluxed for 3 h with stirring. The reaction mixture is added dropwise to 800 mL water/ice with vigorous stirring, stirred for another 30 min and the crude acid chloride E-4a is filtered off. This is dried in vacuo and used further without any purification.

To prepare the acid the crude acid chloride is dissolved in 200 mL THF and 200 mL of 20% aqueous NaHCaO₃ solution are added. The reaction mixture is stirred for 16 h at RT. THF is eliminated in vacuo, the aqueous phase is adjusted to pH 2 with concentrated HCl, stirred for 10 min, the residue formed is suction filtered and washed with water. After drying in vacuo 6.3 g (16.7 mmol, 92%) E-5a is obtained as a colourless solid.

R_{f}=0.24 (silica gel, ethyl acetate)

MS-ESI⁺: 427 (M+H)⁺

### E-4-b) 4-(4-chloro-5-bromo-pyrimidin-2-ylamino)-benzoyl chloride and

### E-5b) 4-(4-chloro-5-bromo-pyrimidin-2-ylamino)-benzoic acid

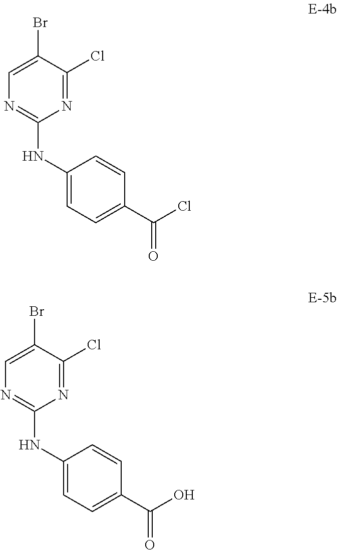 [SMILES: O=C(Cl)c1ccc(Nc2ncc(Br)c(Cl)n2)cc1 ; O=C(O)c1ccc(Nc2ncc(Br)c(Cl)n2)cc1]

Prepared from E-3b analogously to the derivatives E-4a and E-5a.

### E-6b) [4-(5-bromo-4-chloro-pyrimidin-2-ylamino)-phenyl]-(4-methyl-piperazin-1-yl)-methanone

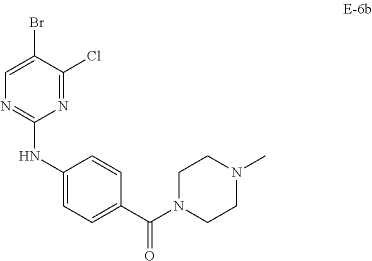 [SMILES: CN1CCN(C(=O)c2ccc(Nc3ncc(Br)c(Cl)n3)cc2)CC1]

559 mg (1.6 mmol) E-4b is dissolved in 5 mL THF and combined with 414 μL (2.4 mmol, 1.5 eq) Hünig base. 181 μL (1.6 mmol, 1 eq) N-methylpiperazine is added dropwise to this solution and the mixture is stirred for 90 min at RT. Then 100 mL water is added and the mixture is extracted 3× with 50 ml ethyl acetate. The combined organic phases are dried on magnesium sulphate and the solvent is eliminated in vacuo. 566 mg (1.4 mmol, 86%) E-6b is obtained in the form of a colourless resin.

MS-ESI⁺: 410/412 (M+H)⁺ (1×Br)

### E-7b) (±)-(1S*,2R*)-2-{5-bromo-2-[4-(4-methyl-piperazin-1-carbonyl)-phenylamino]-pyrimidin-4-ylamino-cyclopentanecarboxylic acid

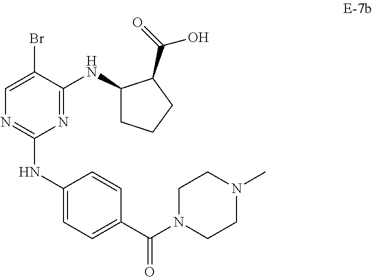 [SMILES: CN1CCN(C(=O)c2ccc(Nc3ncc(Br)c(N[C@@H]4CCC[C@@H]4C(=O)O)n3)cc2)CC1]

459 mg (1.1 mmol) E-6b is dissolved in 5 mL 1-butanol and combined with 536 μL (3.1 mmol, 2.8 eq) Hünig base. 162 mg cis-2-aminocyclopentane-carboxylic acid (racemic) is added to the solution and the reaction mixture is stirred for 100 min at 110° C. (CEM microwave, 100 W). The reaction mixture is evaporated down, stirred into about 200 mL water and extracted 3× with 50 mL ethyl acetate. The combined organic phases are dried on magnesium sulphate and the solvent is eliminated in vacuo. 321 mg (0.64 mmol, 57%) E-7b is obtained in the form of a colourless resin.

MS-ESI⁺: 503/505 (M+H)⁺ (1×Br)

### E-8b) (±)-4-[5-bromo-4-((1R*,2*S)-2-carbamoyl-cyclopentylamino)-pyrimidin-2-ylamino]-benzoic acid

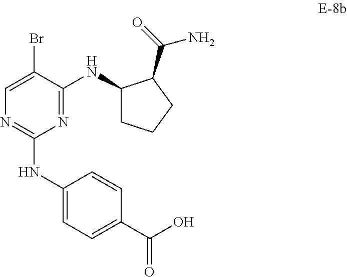 [SMILES: NC(=O)[C@H]1CCC[C@H]1Nc1nc(Nc2ccc(C(=O)O)cc2)ncc1Br]

1 g (3.04 mmol) E-5b is dissolved in 3.9 mL DMA and combined with 1.3 μL (7.6 mmol, 1.5 eq) Hüniig base. 390 mg (3.04 mmol, 1 eq) cis-2-aminocyclopentanecarboxamide (racemic) are added to the solution and the reaction mixture is stirred for 60 min at 120° C.

The reaction mixture is evaporated down, the residue is taken up in 5 ml of 1-butanol and the precipitate is suction filtered. After washing with 5 mL of cold 1-butanol and drying in vacuo, 935 mg (2.2 mmol, 73%) E-8b is obtained in the form of a beige solid.

MS-ESI⁺: 420/422 (M+H)⁺ (1×Br)

The iodine derivative E-8a is prepared analogously from E-5a. The reaction temperature, however, is 80° C.

### E-9b) (±)-4-[4-((1R*,2S*)-2-carbamoyl-cyclopentylamino)-5-cyano-pyrimidin-2-ylamino]-benzoic acid

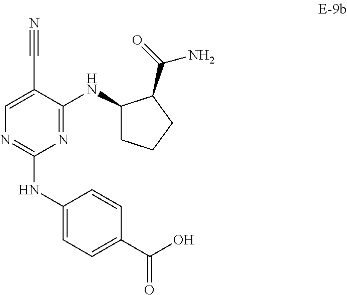 [SMILES: N#Cc1cnc(Nc2ccc(C(=O)O)cc2)nc1N[C@@H]1CCC[C@@H]1C(N)=O]

935 mg (2.23 mmol) E-8b is dissolved in 8 mL DMF and 403 mg (4.45 mmol, 2 eq) copper(I)cyanide is added under argon. The yellow solution is combined with 80 mg (0.067 mmol, 3 mol %) palladium-tetrakistriphenylphosphine and heated to 145° C. for 24 h, during which time about 50% of the educt is reacted. The same amount of catalyst is added again, the mixture is heated for a further 5 h and the reaction is then worked up. The reaction mixture is filtered through a frit filled with silica gel (solvent: DMF), the filtrate is evaporated down to about 5 mL and poured into about 400 mL distilled water. The precipitate formed is filtered off, washed with 100 mL water and dissolved in methanol. RP-gel is added and the solvent is eliminated in vacuo. The mixture is purified by chromatography using a reversed phase (from 5% acetonitrile (+0.2% HCOOH) and 95% water (+0.2% HCOOH) to 50% acetonitrile (+0.2% HCOOH) and 50% water (+0.2% HCOOH)). 160 mg (0.44 mmol, 20%) E-9b is isolated as a beige solid.

R_{f}=0.30 (silica gel, CH₂Cl₂:MeOH:AcOH 5:1:0.1)

MS-ESI⁺: 367 (M+H)⁺

### Example 1

### (±)-(1S*,2R*)-2-{2-[4-(methyl-phenyl-sulphamoyl)-phenylamino]-5-trifluoromethyl-pyrimidin-4-ylamino}-cyclopentanecarbonamide (synthesis scheme A)

150 mg (0.6 mmol) A-2, 519 mg (1.98 mmol, 3 eq) 4-amino-N-methyl-N-phenyl-benzenesulphonamide and 130 μL (0.76 mmol, 1.15 eq) N-ethyldiisopropylamine are dissolved in 3 mL N,N-dimethylacetamide and the solution is stirred for 10 min at 180° C. (heating in the microwave). The solution is stirred into 30 mL water, adjusted to pH 3 with 0.1 N HCl (aq), extracted 3× with 10 mL ethyl acetate, dried on magnesium sulphate and the volatile constituents are eliminated in vacuo. The residue is purified by column chromatography (cyclohexane/ethyl acetate 2/1). 92 mg (0.2 mmol) N-methyl-4-(4-methylsulphanyl-5-trifluoromethyl-pyrimidin-2-ylamino)-N-phenyl-benzenesulphonamide is obtained as a light brown solid.

85 mg (0.19 mmol) of this intermediate is dissolved in 7.5 mL dichloromethane, 64 mg (0.285 mmol, 1.5 eq, 77%) m-chloroperbenzoic acid is added and the mixture is stirred for 3 h at RT. The organic phase is washed 3× with 20 ml saturated aqueous NaHCaO₃ solution and in this way the 3-chlorobenzoic acid is eliminated. After drying the organic phase on sodium sulphate, 83 mg (0.18 mmol, 95%) of 4-(4-methanesulphinyl-5-trifluoromethyl-pyrimidin-2-ylamino)-N-methyl-N-phenyl-benzenesulphonamide (A-4a) is obtained, which is used in the next step without further purification.

83 mg (0.18 mmol) A-4a, 26 mg of cis-2-amino-1-cyclopentanecarboxamide (0.2 mmol, 1.1 eq, racemic) and 35 μL (0.2 mmol, 1.1 eq) of Hünig base are dissolved in 2 mL DMA and stirred for 1 h at 60° C. The reaction mixture is stirred into 10 mL of 0.1 N HCl (aq), the mixture is stirred for 30 min, the precipitate formed is suction filtered, washed with water and dried. Finally, purification is carried out by column chromatography (cHex/EE 60/40 to 50/50 within 20 min). 43 mg (0.08 mmol, 45%) of compound 1 is obtained as a colourless solid.

### Example 2 and 3

### Are Prepared Analogously

### Example 4

### (±)-N-((1S*,2R*)-2-{2-[4-(4-methyl-piperazin-1-carbonyl)-phenylamino]-5-trifluoromethyl-pyrimidin-4-ylamino}-cyclohexyl)-acetamide (Synthesis Scheme C)

38 mg (0.08 mmol) C-3b is dissolved in 50 μL DMA, 25 μL (0.16 mol, 2 eq) Hünig base are added and dissolved for a few minutes at RT. 5 μL acetyl chloride (1 eq) is dissolved in a little DMA and added dropwise to the reaction mixture. After about 10 min the reaction mixture is taken up in dichloromethane, combined with 10 mL RP-gel and all the volatile constituents are eliminated in vacuo. The mixture is purified by chromatography through an RP-phase (AcCN/water 5/95 to 95/5% in 20 min). After the product fractions have been combined and freeze-dried 18 mg (0.034 mmol, 42%) of compound 4 is obtained as a colourless solid.

### Examples 5-12

### Are Prepared Analogously

### Example 13

### (±)-1-methyl-3-((1S*,2R*)-2-{2-[4-(4-methyl-piperazin-1-carbonyl)-phenylamino]-5-trifluoromethyl-pyrimidin-4-ylamino}-cyclohexyl)-urea (Synthesis Scheme C)

50 mg (0.105 mmol) C-3b is dissolved in 50 μL DMF and combined with 55 μL (0.315 mmol, 3 eq) Hünig base. 6 μL methylisocyanate (1 eq) are added to this solution at RT. After about 10 min the reaction mixture is taken up in dichloromethane, combined with 10 mL of RP-gel and all the volatile constituents are eliminated in vacuo. The mixture is purified by chromatography through an RP-phase (AcCN/water 5/95 auf 95/5% in 20 min). After the product fractions have been combined and freeze-dried 24 mg (0.045 mmol, 43%) of compound 13 is obtained as a colourless solid.

### Examples 14-17

### Are Prepared Analogously

### Example 18

### Methyl ((±)-(1S*,2R*)-2-{2-[4-(4-methyl-piperazin-1-carbonyl)-phenylamino]-5-trifluoromethyl-pyrimidin-4-ylamino}-cyclohexyl)-carbamate (Synthesis Scheme C)

30 mg (0.063 mmol) C-3b is dissolved in 50 μL DMF and combined with 22 μL (0.126 mmol, 2 eq) Hünig base. 6 μL methyl chloroformate (1.2 eq) is added to this solution at RT. After about 10 min the reaction mixture is taken up in dichloromethane, combined with 10 mL RP-gel and all the volatile constituents are eliminated in vacuo. The mixture is purified by chromatography through an RP-phase (AcCN/water 5/95 to 95/5% in 20 min). After the product fractions have been combined and freeze-dried 13 mg (0.025 mmol, 39%) of compound 13 is obtained as a colourless solid.

### Examples 19 and 20

### Are Prepared Analogously

### Example 21

### [4-(4-cyclopentylamino-5-trifluoromethyl-pyrimidin-2-ylamino)-phenyl]-(4-methyl-piperazin-1-yl)-methanone (Synthesis Scheme C)

88 mg (0.22 mmol) C-2a is dissolved in 290 μL DMA, 26 μL (0.26 mmol, 1.2 eq) cyclopentylamine and 75 μL (0.44 mmol, 2 eq) Hünig base is added and the reaction mixture is heated to 120° C. After about 90 min the reaction mixture is poured into about 10 mL of distilled water and the precipitate formed is filtered off. The suspension is extracted 3× with 20 mL ethyl acetate, the combined organic phases are dried using saturated aqueous NaCl solution and magnesium sulphate, combined with 100 μL of dioxanic HCl and all the volatile constituents are eliminated in vacuo. 106 mg (0.219 mmol, 99%) of compound 21 is obtained in the form of the hydrochloride.

### Examples 22-26

### Are Prepared Analogously

### Example 27

### (±)-(1S*,2R*)-2-{2-[4-(4-methyl-piperazin-1-carbonyl)-phenylamino]-5-trifluoromethyl-pyrimidin-4-ylamino}-cycloheptanecarboxylic dimethylamide (Synthesis Scheme C)

35 mg (0.067 mmol) C-3d is dissolved in 250 μL DMF, 30 μL (0.175 mmol, 2.6 eq) Hünig base and lastly 35 mg (0.11 mmol, 1.6 eq) TBTU are added. The reaction mixture is stirred for 10 min at RT and then combined with 118 μL dimethylamine (2 M solution in THF, 0.235 mmol, 3.5 eq). The mixture is shaken for 4 h at 35° C., then the reaction mixture is taken up in acetonitrile and combined with 6 mL RP-gel and all the volatile constituents are eliminated in vacuo. The purification is carried out by column chromatography through RP-phase (acetonitrile/water 12/88 to 40/60 in 12 min). The product fractions are freeze-dried and 19 mg (0.035 mmol, 52%) of compound 27 is obtained.

### Examples 28-30

### Are Prepared Analogously

### Example 31

### (±)-4-[4-((1R*,2S*)-2-isopropylcarbamoyl-cyclopentylamino)-5-trifluoromethyl-pyrimidin-2-ylamino]-N-2-(1-methyl-pyrrolidin-2-yl)-ethyl]-benzamide (Synthesis Scheme D)

80 mg (0.18 mmol) D-4c is dissolved in 2.4 mL DMF, 179 μL (1.03 mol, 1.5 eq) Hünig base is added and the solution is combined with 83 mg (0.25 mmol, 1.4 eq) TBTU. The solution is stirred for 40 min at RT, then 38.5 μL (0.27 mmol, 1.5 eq) 2-(2-aminoethyl)-1-methylpyrrolidine is added and the mixture is stirred for 2 days. Then silica gel is added to the reaction mixture and the volatile constituents are eliminated in vacuo. The purification is carried out by column chromatography through a normal phase chromatography (DCM/MeOH/NH₃(aq) 5/1/0.1). 70 mg (0.125 mmol, 70%) of compound 31 is obtained.

### Examples 32-58

### Are Prepared Analogously

### Example 59

### (±)-(1S*,2R*)-2-{2-[4-(4-methyl-piperazin-1-carbonyl)-phenylamino]-5-trifluoromethyl-pyrimidin-4-ylamino}-cyclopentanecarboxylic acid isopropylamide (Synthesis Scheme C)

88 mg (0.18 mmol) C-3d is dissolved in 2 mL DMF, 153 μL (0.90 mmol, 5 eq) of Hünig base is added and the solution is combined with 81 mg (0.25 mmol, 1.4 eq) TBTU. The solution is stirred for 20 min at RT, then 12 μL (0.27 mmol, 1.5 eq) isopropylamine is added and the mixture is stirred for 16 h. It is then filtered through basic aluminium oxide and washed with 20 mL methanol. RP gel is added to the filtrate and the volatile constituents are eliminated in vacuo. The crude product immobilised on the RP-gel is purified through a reversed phase (from 95% water (+0.2% HCOOH) and 5% acetonitrile (+0.2% HCOOH) to 55% water and 45% acetonitrile in 20 min). Corresponding product fractions are combined with 1 eq concentrated hydrochloric acid and freed from the solvent by freeze-drying. 14 mg (0.025 mmol, 14%) of the hydrochloride of compound 59 remain as a colourless film.

### Examples 60-69

### Are Prepared Analogously

Examples 68 and 69 are chiral, and are prepared accordingly from C-2a, using the enantiomers of cis-2-aminocyclopentanecarboxylic acid and lastly forming the isopropylamide prepared. Alternatively 68 and 69 may also be obtained from 59 by preparative chiral HPLC.

### Example 70

### (±)-(1S*,2R*)-2-{2-[3-chloro-4-(4-methyl-piperazin-1-carbonyl)-phenylamino]-5-trifluoromethyl-pyrimidin-4-ylamino}-cyclopentanecarboxylic isopropylamide (Synthesis Scheme B)

30 mg (85.5 mmol) B-2a is dissolved in 100 μL NMP and combined with 35 mg (0.14 mmol, 1.6 eq) (4-amino-2-chloro-phenyl)-(4-methyl-piperazin-1-yl)-methanone. 107 μL of 4 M HCl in dioxane (0.43 mmol, 5 eq) is added to this reaction mixture and it is stirred for 12 h at 5° C. The reaction mixture is taken up in DCM/MeOH/NH₃ 9/1/0.1 and combined with 6 mL RP-gel, the volatile constituents are eliminated in vacuo and purified by chromatography through an RP phase (from 5% acetonitrile to 95% acetonitrile in 10 min). Corresponding product fractions are freed from the solvent by freeze-drying. 35 mg (0.06 mmol, 72%) of compound 70 remain.

### Examples 71-75

### Are Prepared Analogously

### Examples 76-105

### General Method

1 eq of compound B-4 (compound E-8b for Examples 98-101 and compound E-8a for Examples 102-105) is dissolved in DMF (about 1-10 mL per mmol), 4-6 eq Hünig base and then 1.3-1.5 eq TBTU are added. The reaction mixture is stirred for 10-30 min at RT and then 1-1.5 eq of the amine or aniline is added. After the end of the reaction the reaction mixture is combined with silica gel, all the volatile constituents are eliminated in vacuo and the product is purified by column chromatography (normal or RP-phase) and isolated.

### Example 106

### (±)-(3-[4-((1R*,2S*)-2-carbamoyl-cyclopentylamino)-5-trifluoromethyl-pyrimidin-2-ylamino]-N-phenylbenzamide (Synthesis Scheme A)

700 mg (3.06 mmol) A-3 is dissolved in 6 mL DMA. 800 μL (4.6 mmol, 1.5 eq) Hünig base is added and 440 mg cis-2-amino-1-cyclopentanecarboxamide, dissolved in 24 mL DMA, is added dropwise. The reaction mixture is stirred at RT. After 1 h it is diluted with 400 mL dichloromethane and extracted 2× with 200 mL semi-saturated ammonium chloride solution, then dried on magnesium sulphate, and the solvent is eliminated in vacuo. 1.1 g of crude (±)-(1S*,2R*)-2-(2-methylsulphanyl-5-trifluoromethyl-pyrimidin-4-ylamino)-cyclopentanecarboxamide is left as a beige solid. This is reacted further without purification. For this, the solid is dissolved in 60 mL THF, 1.31 g (5.5 mmol, 77% 2 eq) mCPBA is added batchwise and the mixture is stirred for 1 h at RT. The organic phase is washed 3× with 20 ml saturated aqueous sodium hydrogen carbonate solution and in this way the 3-chlorobenzoic acid is eliminated. After drying the organic phase through magnesium sulphate, 1.15 g of crude (±)-(1S*,2R*)-2-(2-methanesulphinyl-5-trifluoromethyl-pyrimidin-4-ylamino)-cyclopentanecarboxamide is obtained, which is used without further purification in the next step. 150 mg (0.45 mmol) of (±)-(1S*,2R*)-2-(2-methanesulphinyl-5-trifluoromethyl-pyrimidin-4-ylamino)-cyclopentanecarboxamide is dissolved in 500 μl NMP, and 148 mg (0.68 mmol, 1.5 eq) m-aminobenzanilide is added. 34 μL hydrochloric acid (4 M solution in dioxane, 0.3 eq) is added to this solution and it is stirred for 16 h at 50° C. The reaction mixture is stirred into 30 mL water, adjusted to pH 3 with 10 mL of 0.1 N HCl and extracted 3× with 15 mL ethyl acetate. The combined organic phases are dried on magnesium sulphate, all the volatile constituents are eliminated in vacuo and the crude product is stirred into cyclohexane/ethyl acetate 60/40, the precipitate is suction filtered and washed with 2-propanol. 15 mg (0.03 mmol, 7%) of compound 106 is obtained as a colourless solid.

### Examples 107-109

### Are Prepared Analogously

Here, the purification is carried out by column chromatography (ethyl acetate/cyclohexane, silica gel).

### Example 110

### (±)-((1S,2R)-2-{5-bromo-2-[4-(4-methyl-piperazin-1-carbonyl)-phenylamino]-pyrimidin-4-ylamino}-cyclopentanecarboxylic acid cyclopropylamide (Synthesis Scheme E)

39 mg (0.077 mmol) E-7b is dissolved in 500 μL DMF, 66 μL (0.39 mmol, 5 eq) Hünig base and 35 mg (0.11 mmol, 1.4 eq) TBTU are added. The solution is stirred for 20 min at RT and then 8 μL (0.116 mmol, 1.5 eq) cyclopropylamine is added and the mixture is overnight at RT. It is filtered through basic aluminium oxide, washed with about 20 mL methanol and the filtrate is combined with 8 mL RP-gel. After elimination of the volatile constituents in vacuo the mixture is purified through a reversed phase (from 95% water (+0.2% HCOOH) and 5% acetonitrile (+0.2% HCOOH) to 5% water and 95% acetonitrile in 20 min). Corresponding product fractions are freed from the solvent by freeze-drying. Compound 110 is obtained as a colourless film, 12 mg (0.021 mmol, 27%).

MS-ESI⁺: 542/544 (M+H)⁺ (1 Br)

### Example 111-120

### Are Prepared Analogously

### Example 121

### N-methyl-N-(1-methyl-piperidin-4-yl)-4-{4-[(±)-(1R*,2S*)-2-(pyrrolidin-1-carbonyl)-cyclopentylamino]-5-trifluoromethyl-pyrimidin-2-ylmino}-benzamide (Synthesis Scheme C)

80 mg (0.15 mmol) C-3e is dissolved in 1.4 mL DMF, 132 μL (0.77 mmol, 5 eq) Hünig base and 69 mg (0.22 mmol, 1.4 eq) TBTU are added. The reaction mixture is stirred for 30 min at RT, then 119 μL (0.144 mmol, 9.4 eq) pyrrolidine is added and the mixture is stirred for 16 h at RT.

It is filtered through basic aluminium oxide, washed with about 20 mL methanol and the filtrate is combined with silica gel. After elimination of the volatile constituents in vacuo, the mixture is purified by column chromatography. (DCM/MeOH/NH₃ 9/1/0.1). After the product fractions have been collected, mixed with 100 μL HCl (4 M solution in dioxane) and the solvent has been eliminated in vacuo, the hydrochloride of compound 121 is obtained as a colourless film, 29 mg (0.048 mmol, 31%).

MS-ESI⁺: 574 (M+H)⁺

### Example 122-128

### Were Prepared Analogously

### Example 129

### 4-[4-((1R,3S)-3-carbamoyl-cyclopentylamino)-5-trifluoromethyl-pyrimidin-2-ylamino]-N-methyl-N-(1-methyl-piperidin-4-yl)-benzamide (Synthesis Scheme C)

75 mg (0.14 mmol) C-3f is dissolved in 1 mL DMF, 123 μl (0.7 mmol, 5 eq) Hünig base is added and the reaction mixture is stirred for 30 min. Then 14 μL (0.22 mmol, 1.5 eq) of aqueous ammonia solution (28%) is added and the mixture is stirred for 5 h at RT. The solution is combined with RP-gel, all the volatile constituents are eliminated in vacuo and the mixture is purified by column chromatography (from 10% acetonitrile (+0.2% HCOOH) and 90% water (+0.2% HCOOH) to 24% acetonitrile and 76% water in 12 min). The product fractions are combined with 100 μL dioxanic HCl and all the volatile constituents are eliminated by freeze-drying. 35 mg (0.063 mol, 44%) of compound 129 are obtained in the form of the hydrochloride.

### Example 130

### Is Prepared Analogously

### Example 131

### (±)-(1S*,2R*)-2-[2-(4-acetylamino-phenylamino)-5-trifluoromethyl-pyrimidin-4-ylamino]-cyclopentanecarboxylic acid isopropylamide (Synthesis Scheme D)

22 mg D-6c is dissolved in 1 mL THF, combined with 14 μL (0.075 mmol, 1.5 eq) Hünig base and then 3 μL acetyl chloride, dissolved in 500 μL THF, is added. After about 90 min the reaction solution is diluted with 10 mL methanol and 8 mL RP-gel is added. Chromatographic purification is carried out through a reversed phase (from 78% water (+0.2% HCOOH) and 22% acetonitrile (+0.2% HCOOH) to 51% water and 49% acetonitrile in 15 min). The corresponding product fractions are combined and the solvent is eliminated by freeze-drying. 14 mg (0.028 mmol, 54%) of compound 131 are obtained.

### Examples 132-133

### Are Prepared Analogously

### Example 134

### (±)-(1S*,2R*)-2-{5-cyano-2-[4-(4-methyl-piperazin-1-carbonyl)-phenylamino]-pyrimidin-4-ylamino}-cyclopentanecarboxamide (Synthesis Scheme E)

40 mg (0.11 mmol) E-9b is dissolved in 1.5 mL DMF, 110 μL (0.63 mmol, 5.8 eq) Hünig base is added and the reaction mixture is stirred for 40 min. Then 18 μL (0.16 mmol, 1.5 eq) N-methylpiperazine is added and the mixture is stirred for 48 h at RT. The solution is combined with silica gel, all the volatile constituents are eliminated in vacuo and the mixture is purified by column chromatography (DCM/MeOH 9/1). 33 mg (0.07 mol, 67%) of compound 134 is obtained.

### Examples 135-136

### Are Prepared Analogously

### Example 137

### (±)-(1S*,2R*)-2-{5-cyclopropylethynyl-2-[4-(4-methyl-piperazin-1-carbonyl)-phenylamino]-pyrimidin-4-ylamino}-cyclopentanecarboxamide (Synthesis Scheme E)

50 mg (0.09 mmol) of 105 is dissolved in 220 μL DMF and then 15 mg of dichloro-bis(triphenylphosphine)palladium (0.021 mmol, 23 mol %) and 10 mg (0.03 mmol, 0.58 eq) copper(I)iodide are added. The solution is combined with 320 μL Hünig base and then with 18 mg (0.27 mmol, 3 eq) ethynylcyclopropane. The reaction mixture is filtered through silica gel with a mixture of DCM/MeOH/NH₃ 4/1/0.1 and then 6 mL RP-gel is added. After elimination of the volatile constituents purification by column chromatography is carried out through a RP-phase (from 95% water (+0.2% HCOOH) and 5% acetonitrile (+0.2% HCOOH) to 50% water and 50% acetonitrile in 20 min). The corresponding product fractions are combined and the solvent is eliminated by freeze-drying. 32 mg (0.065 mmol, 71%) of compound 137 is obtained.

### Examples 138-139

### Are Prepared Analogously

while in Example 138 the reaction is carried out under a propyne atmosphere in a nitrogen flask at 40° C.

### Example 140

### (±)-4-[4-((1R*,2S*)-2-carbamoyl-cyclopentylamino)-5-cyclopropyl-pyrimidin-2-ylamino]-N-(1-methyl-piperidin-4-yl)-benzamide (Synthesis Scheme E)

100 mg (0.15 mmol) 104 is suspended in 1.4 mL dioxane and 13 mg (0.15 mmol, 1 eq) cyclopropylboric acid is added. The solution is degassed in vacuo and 3.5 mg (0.004 mmol, 3 mol %) dichloro[1,1′-bis(diphenylphosphino)-ferrocene]palladium(II)-dichloromethane adduct (PdCl₂dppf DCM) and 2 mL sodium carbonate solution (2 M in water) are added under argon. The two-phase mixture is heated to 130° C. for 5 min (CEM microwave, 100 W). The organic phase is separated off, diluted with methanol and combined with 6 mL RP-gel. After elimination of the volatile constituents purification is carried out by column chromatography through a reversed phase (from 97% water (+0.2% HCOOH) and 3% acetonitrile (+0.2% HCOOH) to 70% water and 30% acetonitrile in 12 minutes v). The corresponding product fractions are combined and the solvent is eliminated by freeze-drying. 2 mg (0.003 mmol, 2%) of compound 140 is obtained.

### Example 141

### (±)-(1S*,2R*)-2-[2-(4-[1.4]diazepan-1-yl-3-fluoro-phenylamino)-5-trifluoromethyl-pyrimidin-4-ylamino]-cyclopentanecarboxylic acid isopropylamide (Synthesis Scheme B)

23 mg (0.066 mmol) B-2a is dissolved in 100 μL NMP, 17 mg (0.079 mmol, 1.2 eq) 3-fluoro-4-(4-methyl-[1.4]diazepan-1-yl)-phenylamine and finally 46 μL HCl (0.18 mmol, 2.8 eq, 4 M solution in dioxane) are added. The reaction mixture is heated to 90° C. for 12 h, combined with 6 mL RP-gel and the volatile constituents are eliminated in vacuo. Chromatographic purification is carried out through a reversed phase (from 95% water (+0.2% HCOOH) and 5% acetonitrile (+0.2% HCOOH) to 55% water and 45% acetonitrile in 25 min). The corresponding product fractions are combined and the solvent is eliminated by freeze-drying. 3 mg (0.005 mmol, 8%) of compound 141 is obtained.

### Examples 142-144

### Are Prepared Analogously

### Example 145

### (±)-(1R*,2R*)-2-{2-[4-(4-methyl-piperazin-1-carbonyl)-phenylamino]-5-trifluoromethyl-pyrimidin-4-ylamino}-cyclopentanecarboxamide (Synthesis Scheme C)

100 mg (0.25 mmol) C-2a is dissolved in 1 mL 1-butanol and this solution is combined with 35 mg (0.275 mmol, 1.1 eq) racemic trans-2-aminocyclopentanecarboxamide and 60 μL (0.35 mmol, 1.4 eq) Hünig base. At 110° C. (100 W, microwave CEM) the mixture is stirred for 30 min until complete conversion is obtained. About 20 mL methanol is added to the reaction mixture, this is combined with RP-gel (about 8 mL) and all the volatile constituents are eliminated in vacuo. The mixture is purified through an RP column (from 95% water (+0.2% HCOOH) and 5% acetonitrile (+0.2% HCOOH) to 55% water and 45% acetonitrile in 20 min). Corresponding product fractions are combined with concentrated hydrochloric acid and freed from the solvent by freeze-drying. 77 mg (0.146 mmol, 58%) of compound 145 is obtained as a colourless solid.

### Examples 146-147

### Are Prepared Analogously

while Example 148 is prepared analogously to Example 129 (nucleophilic substitution with the β-amino acid starting from C-2a and finally amide linking with ammonia).

### Examples 1-148

| | | | | | HPLC | MS | |
|---|---|---|---|---|---|---|---|
| Ex. | | R_{f}/ | | m.p. | RT | (ESI⁺) | UVₘₐₓ |
| no. | structure | eluant | | [° C.] | [min] | [M + H]⁺ | [nm] |
| 1 | 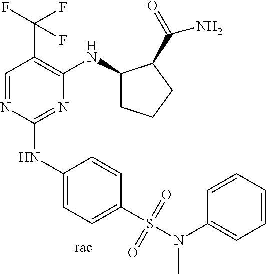 [SMILES: CN(c1ccccc1)S(=O)(=O)c1ccc(Nc2ncc(C(F)(F)F)c(N[C@@H]3CCC[C@@H]3C(N)=O)n2)cc1] | 0.32 | EE:cHex 1:1 | | 2.20 | 535 | 306 |
| 2 | 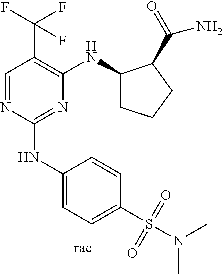 [SMILES: CN(C)S(=O)(=O)c1ccc(Nc2ncc(C(F)(F)F)c(N[C@@H]3CCC[C@@H]3C(N)=O)n2)cc1] | 0.20 | EE:cHex 1:1 | | | 473 | |
| 3 | 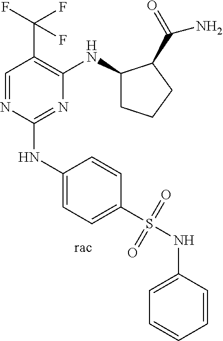 [SMILES: NC(=O)[C@H]1CCC[C@H]1Nc1nc(Nc2ccc(S(=O)(=O)Nc3ccccc3)cc2)ncc1C(F)(F)F] | 0.20 | EE:cHex 1:1 | | | 521 | |
| 4 | 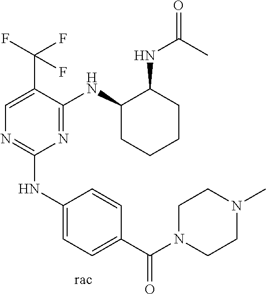 [SMILES: CC(=O)N[C@H]1CCCC[C@H]1Nc1nc(Nc2ccc(C(=O)N3CCN(C)CC3)cc2)ncc1C(F)(F)F] | 0.46 | DCM:MeOH: NH₃ 9:1:0.1 | | 1.41 | 520 | 276 |
| 5 | 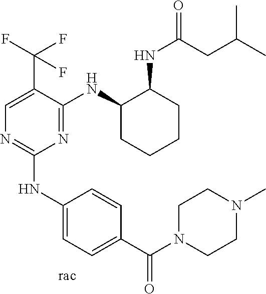 [SMILES: CC(C)CC(=O)N[C@H]1CCCC[C@H]1Nc1nc(Nc2ccc(C(=O)N3CCN(C)CC3)cc2)ncc1C(F)(F)F] | 0.30 | DCM:MeOH: NH₃ 9:1:0.1 | | 1.51 | 562 | 279 |
| 6 | 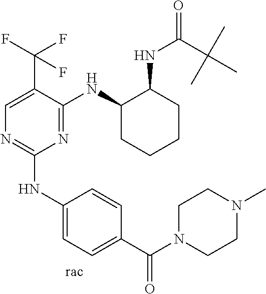 [SMILES: CN1CCN(C(=O)c2ccc(Nc3ncc(C(F)(F)F)c(N[C@@H]4CCCC[C@@H]4NC(=O)C(C)(C)C)n3)cc2)CC1] | 0.39 | DCM:MeOH: NH₃ 9:1:0.1 | | 1.54 | 562 | 280 |
| 7 | 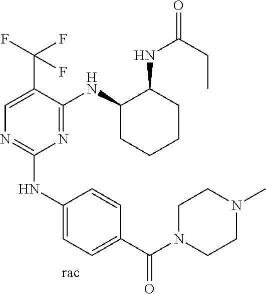 [SMILES: CCC(=O)N[C@H]1CCCC[C@H]1Nc1nc(Nc2ccc(C(=O)N3CCN(C)CC3)cc2)ncc1C(F)(F)F] | 0.39 | DCM:MeOH: NH₃ 9:1:0.1 | | 1.53 | 534 | 279 |
| 8 | 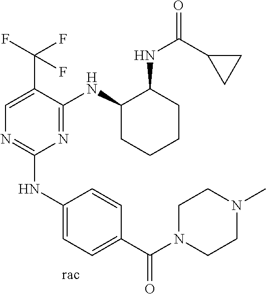 [SMILES: CN1CCN(C(=O)c2ccc(Nc3ncc(C(F)(F)F)c(N[C@@H]4CCCC[C@@H]4NC(=O)C4CC4)n3)cc2)CC1] | 0.29 | DCM:MeOH: NH₃ 9:1:0.1 | | 1.59 | 549 | 279 |
| 9 | 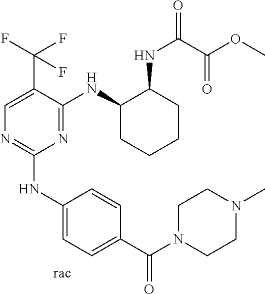 [SMILES: COC(=O)C(=O)N[C@H]1CCCC[C@H]1Nc1nc(Nc2ccc(C(=O)N3CCN(C)CC3)cc2)ncc1C(F)(F)F] | 0.34 | DCM:MeOH: NH₃ 9:1:0.1 | | 1.60 | 564 | 280/296 |
| 10 | 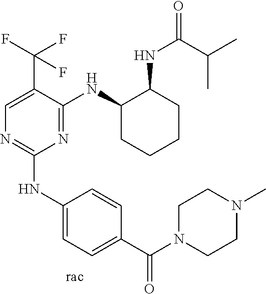 [SMILES: CC(C)C(=O)N[C@H]1CCCC[C@H]1Nc1nc(Nc2ccc(C(=O)N3CCN(C)CC3)cc2)ncc1C(F)(F)F] | 0.34 | DCM:MeOH: NH₃ 9:1:0.1 | | 1.62 | 548 | 279 |
| 11 | 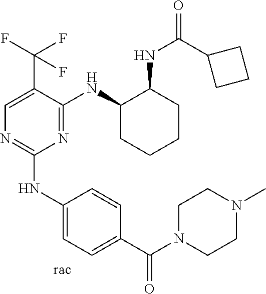 [SMILES: CN1CCN(C(=O)c2ccc(Nc3ncc(C(F)(F)F)c(N[C@@H]4CCCC[C@@H]4NC(=O)C4CCC4)n3)cc2)CC1] | 0.32 | DCM:MeOH: NH₃ 9:1:0.1 | | 1.40 | 560 | 280 |
| 12 | 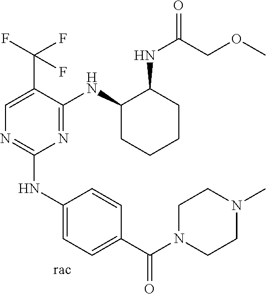 [SMILES: COCC(=O)N[C@H]1CCCC[C@H]1Nc1nc(Nc2ccc(C(=O)N3CCN(C)CC3)cc2)ncc1C(F)(F)F] | 0.32 | DCM:MeOH: NH₃ 9:1:0.1 | | 1.55 | 550 | 279 |
| 13 | 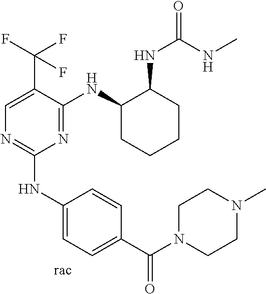 [SMILES: CNC(=O)N[C@H]1CCCC[C@H]1Nc1nc(Nc2ccc(C(=O)N3CCN(C)CC3)cc2)ncc1C(F)(F)F] | 0.16 | DCM:MeOH: NH₃ 9:1:0.1 | | 1.44 | 535 | 277 |
| 14 | 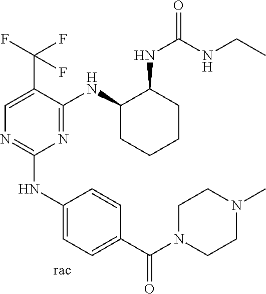 [SMILES: CCNC(=O)N[C@H]1CCCC[C@H]1Nc1nc(Nc2ccc(C(=O)N3CCN(C)CC3)cc2)ncc1C(F)(F)F] | 0.39 | DCM:MeOH: NH₃ 9:1:0.1 | | 1.52 | 549 | 278 |
| 15 | 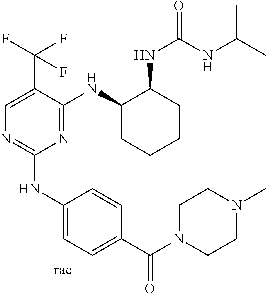 [SMILES: CC(C)NC(=O)N[C@H]1CCCC[C@H]1Nc1nc(Nc2ccc(C(=O)N3CCN(C)CC3)cc2)ncc1C(F)(F)F] | 0.36 | DCM:MeOH: NH₃ 9:1:0.1 | 153- 156 | 1.31 | 563 | 276 |
| 16 | 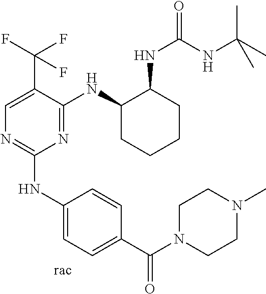 [SMILES: CN1CCN(C(=O)c2ccc(Nc3ncc(C(F)(F)F)c(N[C@@H]4CCCC[C@@H]4NC(=O)NC(C)(C)C)n3)cc2)CC1] | 0.35 | DCM:MeOH: NH₃ 9:1:0.1 | | 1.49 | 577 | 277 |
| 17 | 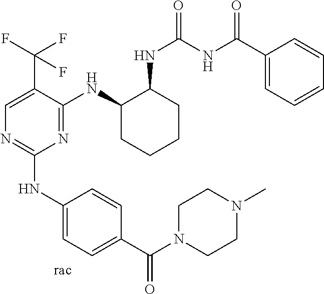 [SMILES: CN1CCN(C(=O)c2ccc(Nc3ncc(C(F)(F)F)c(N[C@@H]4CCCC[C@@H]4NC(=O)NC(=O)c4ccccc4)n3)cc2)CC1] | 0.43 | DCM:MeOH: NH₃ 9:1:0.1 | 137- 139 | 1.67 | 625 | 280/298 |
| 18 | 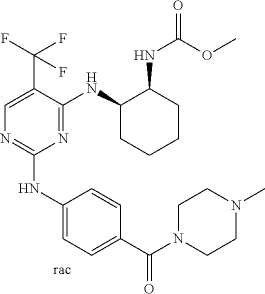 [SMILES: COC(=O)N[C@H]1CCCC[C@H]1Nc1nc(Nc2ccc(C(=O)N3CCN(C)CC3)cc2)ncc1C(F)(F)F] | 0.44 | DCM:MeOH: NH₃ 9:1:0.1 | | 1.30 | 536 | 277 |
| 19 | 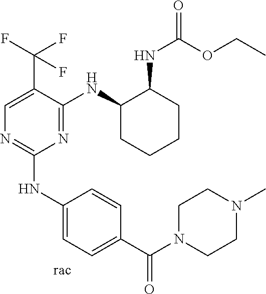 [SMILES: CCOC(=O)N[C@H]1CCCC[C@H]1Nc1nc(Nc2ccc(C(=O)N3CCN(C)CC3)cc2)ncc1C(F)(F)F] | 0.53 | DCM:MeOH: NH₃ 9:1:0.1 | | 1.34 | 550 | 277 |
| 20 | 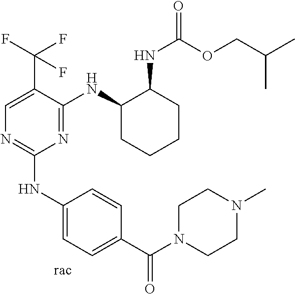 [SMILES: CC(C)COC(=O)N[C@H]1CCCC[C@H]1Nc1nc(Nc2ccc(C(=O)N3CCN(C)CC3)cc2)ncc1C(F)(F)F] | 0.42 | DCM:MeOH: NH₃ 9:1:0.1 | | 1.65 | 578 | 281/ 297 |
| 21 | 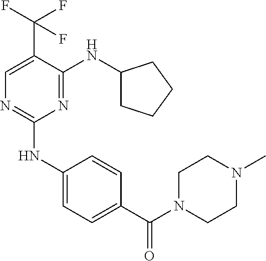 [SMILES: CN1CCN(C(=O)c2ccc(Nc3ncc(C(F)(F)F)c(NC4CCCC4)n3)cc2)CC1] | 0.52 | DCM:MeOH: NH₃ 5:1:0.1 | | 1.49 | 449 | 277 |
| 22 | 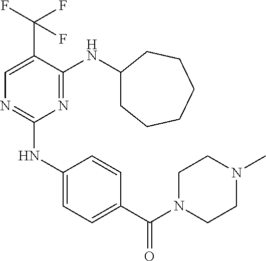 [SMILES: CN1CCN(C(=O)c2ccc(Nc3ncc(C(F)(F)F)c(NC4CCCCCC4)n3)cc2)CC1] | 0.50 | DCM:MeOH: NH₃ 5:1:0.1 | | 1.58 | 477 | 278 |
| 23 | 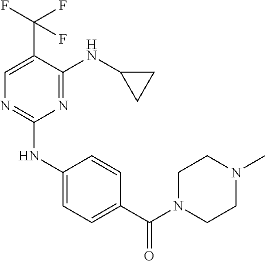 [SMILES: CN1CCN(C(=O)c2ccc(Nc3ncc(C(F)(F)F)c(NC4CC4)n3)cc2)CC1] | 0.48 | DCM:MeOH: NH₃ 5:1:0.1 | | 1.33 | 421 | 279 |
| 24 | 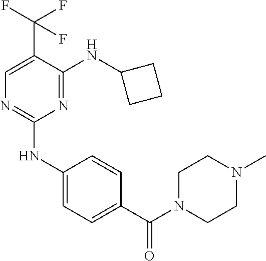 [SMILES: CN1CCN(C(=O)c2ccc(Nc3ncc(C(F)(F)F)c(NC4CCC4)n3)cc2)CC1] | 0.49 | DCM:MeOH: NH₃ 5:1:0.1 | | 1.52 | 435 | 2.78 |
| 25 | 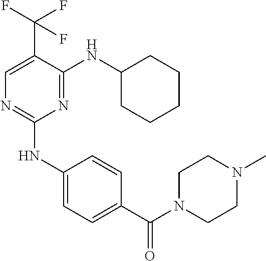 [SMILES: CN1CCN(C(=O)c2ccc(Nc3ncc(C(F)(F)F)c(NC4CCCCC4)n3)cc2)CC1] | 0.55 | DCM:MeOH: NH₃ 5:1:0.1 | | 1.47 | 463 | 277 |
| 26 | 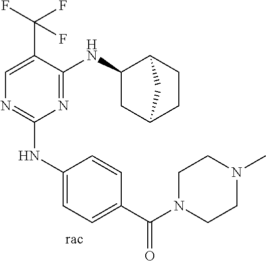 [SMILES: CN1CCN(C(=O)c2ccc(Nc3ncc(C(F)(F)F)c(N[C@@H]4C[C@@H]5CC[C@H]4C5)n3)cc2)CC1] | 0.50 | DCM:MeOH: NH₃ 5:1:0.1 | | 1.61 | 475 | 279 |
| 27 | 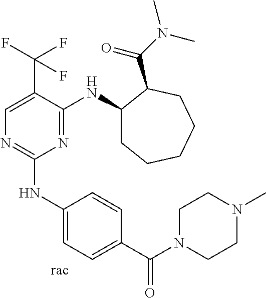 [SMILES: CN1CCN(C(=O)c2ccc(Nc3ncc(C(F)(F)F)c(N[C@@H]4CCCCC[C@@H]4C(=O)N(C)C)n3)cc2)CC1] | 0.39 | DCM:MeOH: NH₃ 9:1:0.1 | | 1.63 | 548 | 279 |
| 28 | 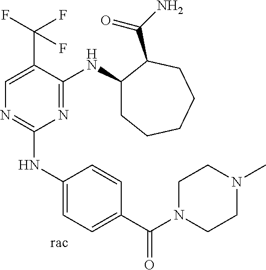 [SMILES: CN1CCN(C(=O)c2ccc(Nc3ncc(C(F)(F)F)c(N[C@@H]4CCCCC[C@@H]4C(N)=O)n3)cc2)CC1] | 0.25 | DCM:MeOH: NH₃ 9:1:0.1 | 138- 141 | 1.49 | 520 | 278 |
| 29 | 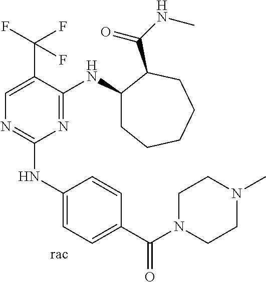 [SMILES: CNC(=O)[C@H]1CCCCC[C@H]1Nc1nc(Nc2ccc(C(=O)N3CCN(C)CC3)cc2)ncc1C(F)(F)F] | 0.26 | DCM:MeOH: NH₃ 9:1:0.1 | | 1.55 | 534 | 278 |
| 30 | 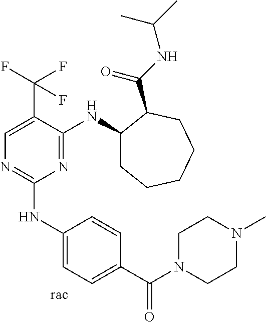 [SMILES: CC(C)NC(=O)[C@H]1CCCCC[C@H]1Nc1nc(Nc2ccc(C(=O)N3CCN(C)CC3)cc2)ncc1C(F)(F)F] | 0.30 | DCM:MeOH: NH₃ 9:1:0.1 | | 1.54 | 562 | 279 |
| 31 | 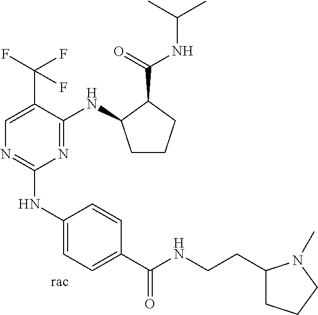 [SMILES: CC(C)NC(=O)[C@H]1CCC[C@H]1Nc1nc(Nc2ccc(C(=O)NCCC3CCCN3C)cc2)ncc1C(F)(F)F] | 0.06 | DCM:MeOH: NH₃ 5:1:0.1 | | 1.47 | 562 | 297 |
| 32 | 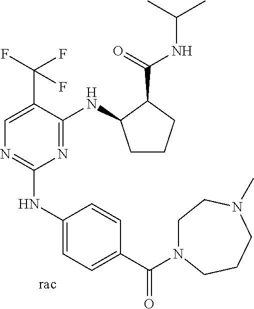 [SMILES: CC(C)NC(=O)[C@H]1CCC[C@H]1Nc1nc(Nc2ccc(C(=O)N3CCCN(C)CC3)cc2)ncc1C(F)(F)F] | 0.25 | DCM:MeOH: NH₃ 5:1:0.1 | | 1.30 | 548 | 276 |
| 33 | 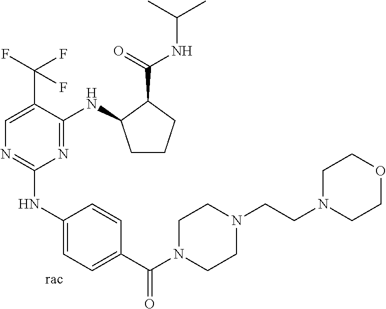 [SMILES: CC(C)NC(=O)[C@H]1CCC[C@H]1Nc1nc(Nc2ccc(C(=O)N3CCN(CCN4CCOCC4)CC3)cc2)ncc1C(F)(F)F] | 0.35 | DCM:MeOH: NH₃ 5:1:0.1 | | 1.35 | 633 | 277 |
| 34 | 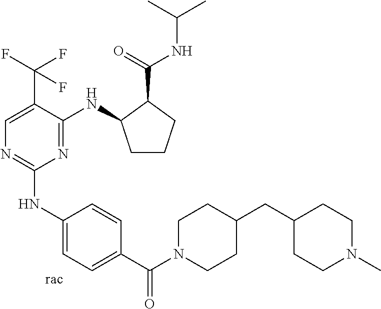 [SMILES: CC(C)NC(=O)[C@H]1CCC[C@H]1Nc1nc(Nc2ccc(C(=O)N3CCC(CC4CCN(C)CC4)CC3)cc2)ncc1C(F)(F)F] | 0.04 | DCM:MeOH: NH₃ 5:1:0.1 | | 1.42 | 631 | 281 |
| 35 | 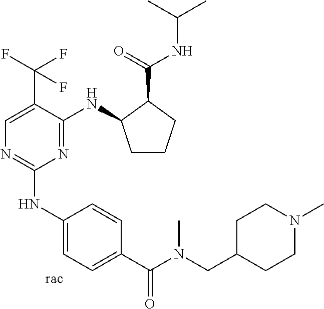 [SMILES: CC(C)NC(=O)[C@H]1CCC[C@H]1Nc1nc(Nc2ccc(C(=O)N(C)CC3CCN(C)CC3)cc2)ncc1C(F)(F)F] | 0.09 | DCM:MeOH: NH₃ 5:1:0.1 | | 1.45 | 576 | 276 |
| 36 | 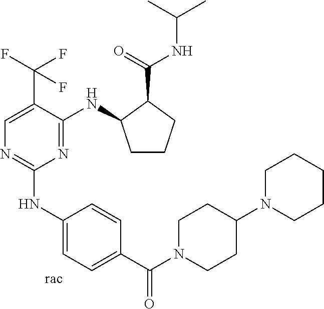 [SMILES: CC(C)NC(=O)[C@H]1CCC[C@H]1Nc1nc(Nc2ccc(C(=O)N3CCC(N4CCCCC4)CC3)cc2)ncc1C(F)(F)F] | 0.47 | DCM:MeOH: NH₃ 5:1:0.1 | | 1.49 | 602 | 278 |
| 37 | 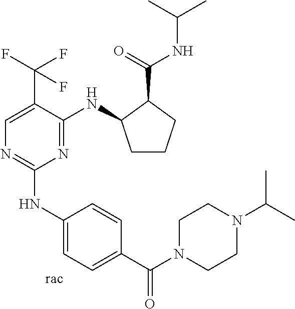 [SMILES: CC(C)NC(=O)[C@H]1CCC[C@H]1Nc1nc(Nc2ccc(C(=O)N3CCN(C(C)C)CC3)cc2)ncc1C(F)(F)F] | 0.63 | DCM:MeOH: NH₃ 5:1:0.1 | | 1.34 | 562 | 278 |
| 38 | 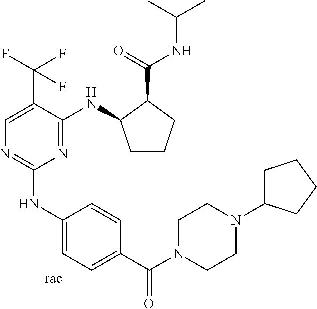 [SMILES: CC(C)NC(=O)[C@H]1CCC[C@H]1Nc1nc(Nc2ccc(C(=O)N3CCN(C4CCCC4)CC3)cc2)ncc1C(F)(F)F] | 0.58 | DCM:MeOH: NH₃ 5:1:0.1 | | 1.55 | 588 | 280 |
| 39 | 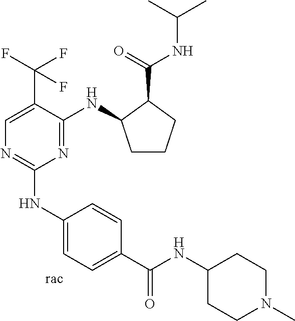 [SMILES: CC(C)NC(=O)[C@H]1CCC[C@H]1Nc1nc(Nc2ccc(C(=O)NC3CCN(C)CC3)cc2)ncc1C(F)(F)F] | 0.41 | DCM:MeOH: NH₃ 5:1:0.1 | | 1.42 | 548 | 288 |
| 40 | 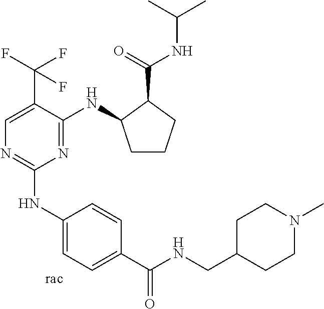 [SMILES: CC(C)NC(=O)[C@H]1CCC[C@H]1Nc1nc(Nc2ccc(C(=O)NCC3CCN(C)CC3)cc2)ncc1C(F)(F)F] | 0.24 | DCM:MeOH: NH₃ 5:1:0.1 | | 1.43 | 562 | 287 |
| 41 | 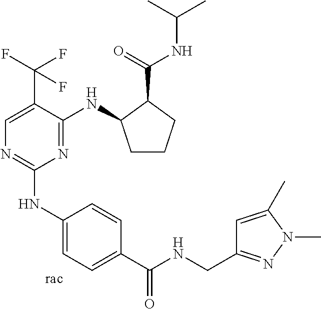 [SMILES: Cc1cc(CNC(=O)c2ccc(Nc3ncc(C(F)(F)F)c(N[C@@H]4CCC[C@@H]4C(=O)NC(C)C)n3)cc2)nn1C] | 0.63 | DCM:MeOH: NH₃ 5:1:0.1 | | 1.98 | 559 | 305 |
| 42 | 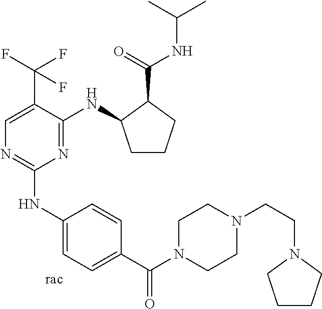 [SMILES: CC(C)NC(=O)[C@H]1CCC[C@H]1Nc1nc(Nc2ccc(C(=O)N3CCN(CCN4CCCC4)CC3)cc2)ncc1C(F)(F)F] | 0.39 | DCM:MeOH: NH₃ 5:1:0.1 | | 1.36 | 617 | 277 |
| 43 | 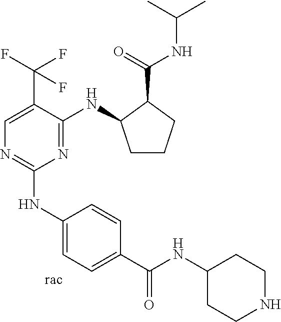 [SMILES: CC(C)NC(=O)[C@H]1CCC[C@H]1Nc1nc(Nc2ccc(C(=O)NC3CCNCC3)cc2)ncc1C(F)(F)F] | 0.10 | DCM:MeOH: NH₃ 5:1:0.1 | | 1.42 | 534 | 288 |
| 44 | 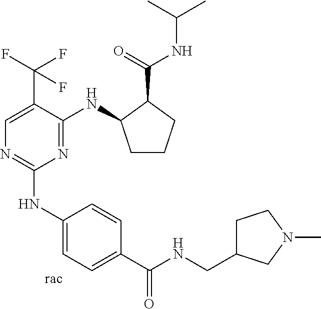 [SMILES: CC(C)NC(=O)[C@H]1CCC[C@H]1Nc1nc(Nc2ccc(C(=O)NCC3CCN(C)C3)cc2)ncc1C(F)(F)F] | 0.26 | DCM:MeOH: NH₃ 5:1:0.1 | | 1.47 | 548 | 298 |
| 45 | 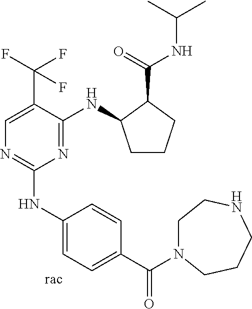 [SMILES: CC(C)NC(=O)[C@H]1CCC[C@H]1Nc1nc(Nc2ccc(C(=O)N3CCCNCC3)cc2)ncc1C(F)(F)F] | 0.45 | DCM:MeOH: NH₃ 5:1:0.1 | | 1.31 | 534 | 276 |
| 46 | 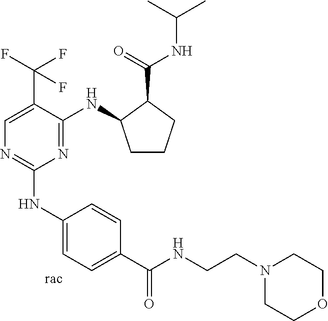 [SMILES: CC(C)NC(=O)[C@H]1CCC[C@H]1Nc1nc(Nc2ccc(C(=O)NCCN3CCOCC3)cc2)ncc1C(F)(F)F] | 0.64 | DCM:MeOH: NH₃ 5:1:0.1 | 131- 134 | 1.49 | 564 | 304 |
| 47 | 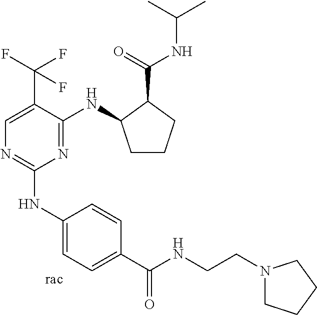 [SMILES: CC(C)NC(=O)[C@H]1CCC[C@H]1Nc1nc(Nc2ccc(C(=O)NCCN3CCCC3)cc2)ncc1C(F)(F)F] | 0.53 | DCM:MeOH: NH₃ 5:1:0.1 | 123- 126 | 1.49 | 548 | 303 |
| 48 | 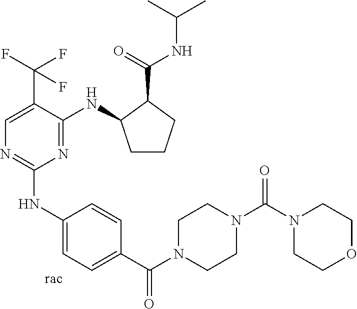 [SMILES: CC(C)NC(=O)[C@H]1CCC[C@H]1Nc1nc(Nc2ccc(C(=O)N3CCN(C(=O)N4CCOCC4)CC3)cc2)ncc1C(F)(F)F] | 0.80 | DCM:MeOH: NH₃ 5:1:0.1 | | 1.88 | 633 | 279 |
| 49 | 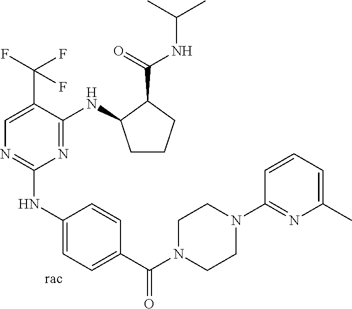 [SMILES: Cc1cccc(N2CCN(C(=O)c3ccc(Nc4ncc(C(F)(F)F)c(N[C@@H]5CCC[C@@H]5C(=O)NC(C)C)n4)cc3)CC2)n1] | 0.70 | DCM:MeOH: NH₃ 5:1:0.1 | | 1.68 | 611 | 304 |
| 50 | 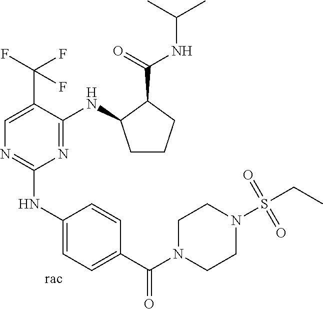 [SMILES: CCS(=O)(=O)N1CCN(C(=O)c2ccc(Nc3ncc(C(F)(F)F)c(N[C@@H]4CCC[C@@H]4C(=O)NC(C)C)n3)cc2)CC1] | 0.34 | DCM:MeOH: NH₃ 5:1:0.1 | | 2.02 | 612 | 280 |
| 51 | 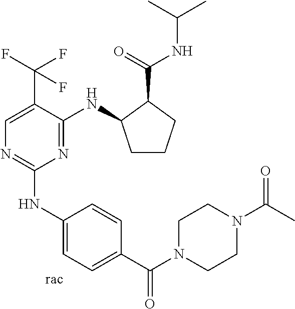 [SMILES: CC(=O)N1CCN(C(=O)c2ccc(Nc3ncc(C(F)(F)F)c(N[C@@H]4CCC[C@@H]4C(=O)NC(C)C)n3)cc2)CC1] | 0.78 | DCM:MeOH: NH₃ 5:1:0.1 | | 1.81 | 562 | 279 |
| 52 | 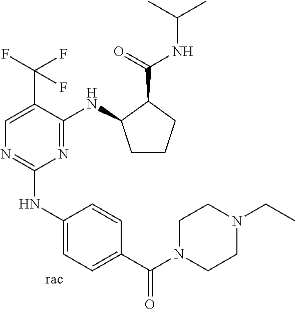 [SMILES: CCN1CCN(C(=O)c2ccc(Nc3ncc(C(F)(F)F)c(N[C@@H]4CCC[C@@H]4C(=O)NC(C)C)n3)cc2)CC1] | 0.68 | DCM:MeOH: NH₃ 5:1:0.1 | | 1.30 | 548 | 279 |
| 53 | 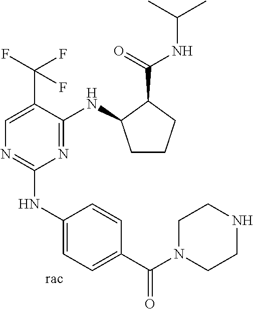 [SMILES: CC(C)NC(=O)[C@H]1CCC[C@H]1Nc1nc(Nc2ccc(C(=O)N3CCNCC3)cc2)ncc1C(F)(F)F] | 0.10 | DCM:MeOH: NH₃ 5:1:0.1 | | 1.52 | 520 | 279 |
| 54 | 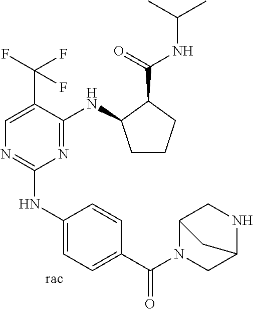 [SMILES: CC(C)NC(=O)[C@H]1CCC[C@H]1Nc1nc(Nc2ccc(C(=O)N3CC4CC3CN4)cc2)ncc1C(F)(F)F] | 0.16 | DCM:MeOH: NH₃ 5:1:0.1 | | 1.30 | 532 | 280 |
| 55 | 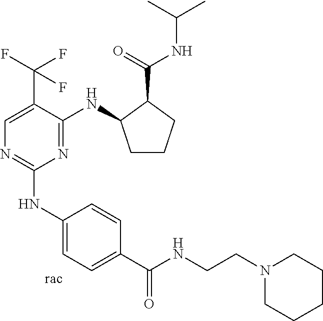 [SMILES: CC(C)NC(=O)[C@H]1CCC[C@H]1Nc1nc(Nc2ccc(C(=O)NCCN3CCCCC3)cc2)ncc1C(F)(F)F] | 0.67 | DCM:MeOH: NH₃ 5:1:0.1 | 126- 129 | 1.53 | 562 | 34 |
| 56 | 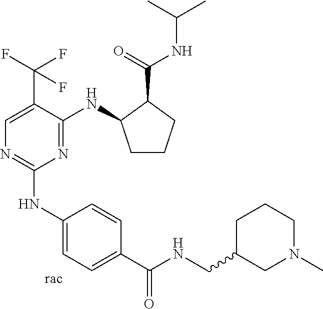 [SMILES: CC(C)NC(=O)[C@H]1CCC[C@H]1Nc1nc(Nc2ccc(C(=O)NCC3CCCN(C)C3)cc2)ncc1C(F)(F)F] | | | | 1.47 | 562 | 298 |
| 57 | 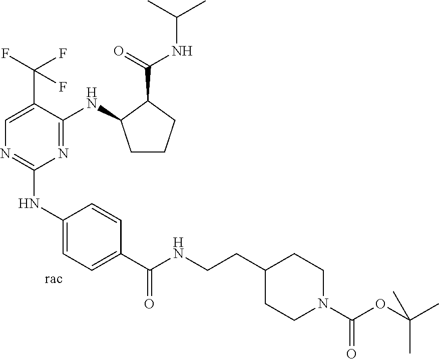 [SMILES: CC(C)NC(=O)[C@H]1CCC[C@H]1Nc1nc(Nc2ccc(C(=O)NCCC3CCN(C(=O)OC(C)(C)C)CC3)cc2)ncc1C(F)(F)F] | | | | 2.43 | 662 | 306 |
| 58 | 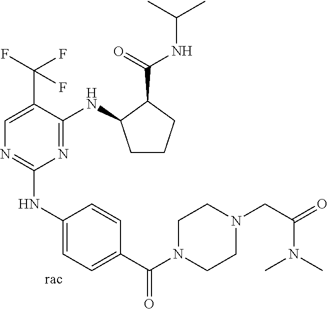 [SMILES: CC(C)NC(=O)[C@H]1CCC[C@H]1Nc1nc(Nc2ccc(C(=O)N3CCN(CC(=O)N(C)C)CC3)cc2)ncc1C(F)(F)F] | 0.69 | DCM:MeOH: NH₃ 5:1:0.1 | | 1.40 | 605 | 279 |
| 59 | 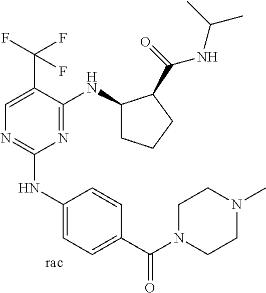 [SMILES: CC(C)NC(=O)[C@H]1CCC[C@H]1Nc1nc(Nc2ccc(C(=O)N3CCN(C)CC3)cc2)ncc1C(F)(F)F] | 0.46 | DCM:MeOH: NH₃ 5:1:0.1 | | 1.57 | 534 | 279 |
| 60 | 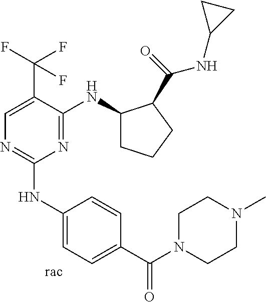 [SMILES: CN1CCN(C(=O)c2ccc(Nc3ncc(C(F)(F)F)c(N[C@@H]4CCC[C@@H]4C(=O)NC4CC4)n3)cc2)CC1] | 0.58 | DCM:MeOH: NH₃ 5:1:0.1 | | 1.51 | 532 | 280 |
| 61 | 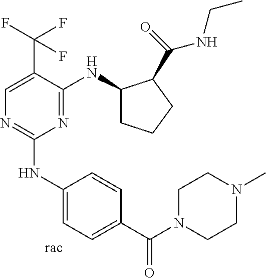 [SMILES: CCNC(=O)[C@H]1CCC[C@H]1Nc1nc(Nc2ccc(C(=O)N3CCN(C)CC3)cc2)ncc1C(F)(F)F] | 0.55 | DCM:MeOH: NH₃ 5:1:0.1 | | 1.48 | 520 | 279 |
| 62 | 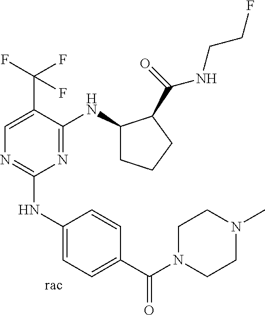 [SMILES: CN1CCN(C(=O)c2ccc(Nc3ncc(C(F)(F)F)c(N[C@@H]4CCC[C@@H]4C(=O)NCCF)n3)cc2)CC1] | 0.54 | DCM:MeOH: NH₃ 5:1:0.1 | | 1.50 | 538 | 279 |
| 63 | 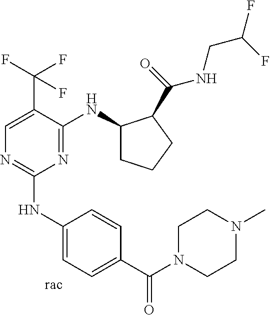 [SMILES: CN1CCN(C(=O)c2ccc(Nc3ncc(C(F)(F)F)c(N[C@@H]4CCC[C@@H]4C(=O)NCC(F)F)n3)cc2)CC1] | 0.59 | DCM:MeOH: NH₃ 5:1:0.1 | | 1.58 | 556 | 280 |
| 64 | 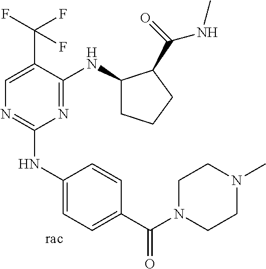 [SMILES: CNC(=O)[C@H]1CCC[C@H]1Nc1nc(Nc2ccc(C(=O)N3CCN(C)CC3)cc2)ncc1C(F)(F)F] | 0.63 | DCM:MeOH: NH₃ 5:1:0.1 | | 1.39 | 506 | 278 |
| 65 | 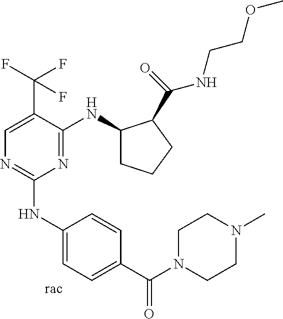 [SMILES: COCCNC(=O)[C@H]1CCC[C@H]1Nc1nc(Nc2ccc(C(=O)N3CCN(C)CC3)cc2)ncc1C(F)(F)F] | 0.62 | DCM:MeOH: NH₃ 5:1:0.1 | | 1.48 | 550 | 279 |
| 66 | 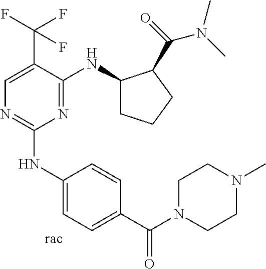 [SMILES: CN1CCN(C(=O)c2ccc(Nc3ncc(C(F)(F)F)c(N[C@@H]4CCC[C@@H]4C(=O)N(C)C)n3)cc2)CC1] | 0.62 | DCM:MeOH: NH₃ 5:1:0.1 | | 1.37 | 520 | 299 |
| 67 | 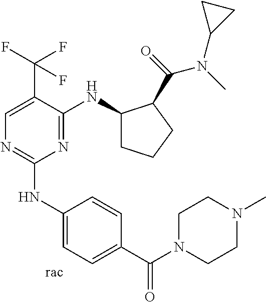 [SMILES: CN1CCN(C(=O)c2ccc(Nc3ncc(C(F)(F)F)c(N[C@@H]4CCC[C@@H]4C(=O)N(C)C4CC4)n3)cc2)CC1] | 0.64 | DCM:MeOH: NH₃ 5:1:0.1 | | 1.30 | 546 | 276 |
| 68 | 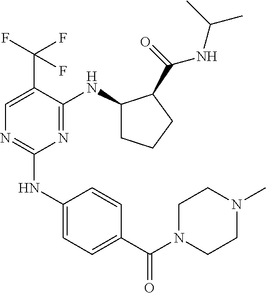 [SMILES: CC(C)NC(=O)[C@H]1CCC[C@H]1Nc1nc(Nc2ccc(C(=O)N3CCN(C)CC3)cc2)ncc1C(F)(F)F] | 0.46 | DCM:MeOH: NH₃ 5:1:0.1 | 189- 192 | 1.40 | 534 | 279 |
| 69 | 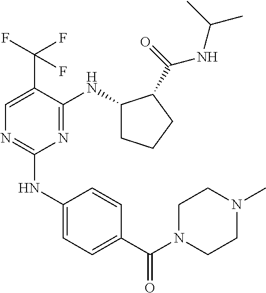 [SMILES: CC(C)NC(=O)[C@@H]1CCC[C@@H]1Nc1nc(Nc2ccc(C(=O)N3CCN(C)CC3)cc2)ncc1C(F)(F)F] | 0.46 | DCM:MeOH: NH₃ 5:1:0.1 | | 1.40 | 534 | 279 |
| 70 | 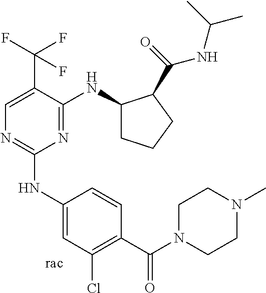 [SMILES: CC(C)NC(=O)[C@H]1CCC[C@H]1Nc1nc(Nc2ccc(C(=O)N3CCN(C)CC3)c(Cl)c2)ncc1C(F)(F)F] | 0.35 | DCM:MeOH: NH₃ 9:1:0.1 | | 1.47 | 568/570 (1 Cl) | 274 |
| 71 | 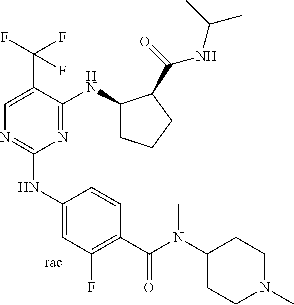 [SMILES: CC(C)NC(=O)[C@H]1CCC[C@H]1Nc1nc(Nc2ccc(C(=O)N(C)C3CCN(C)CC3)c(F)c2)ncc1C(F)(F)F] | 0.15 | DCM:MeOH: NH₃ 9:1:0.1 | | 1.54 | 580 | 272/297 |
| 72 | 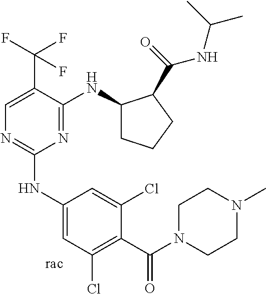 [SMILES: CC(C)NC(=O)[C@H]1CCC[C@H]1Nc1nc(Nc2cc(Cl)c(C(=O)N3CCN(C)CC3)c(Cl)c2)ncc1C(F)(F)F] | 0.43 | DCM:MeOH: NH₃ 9:1:0.1 | | 1.60 | 602/604 (2 Cl) | 275/299 |
| 73 | 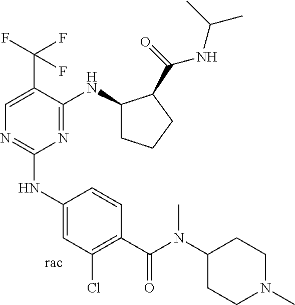 [SMILES: CC(C)NC(=O)[C@H]1CCC[C@H]1Nc1nc(Nc2ccc(C(=O)N(C)C3CCN(C)CC3)c(Cl)c2)ncc1C(F)(F)F] | 0.14 | DCM:MeOH: NH₃ 9:1:0.1 | | 1.52 | 582/584 (1 Cl) | 276/298 |
| 74 | 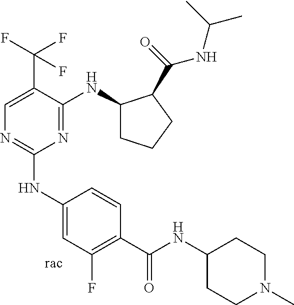 [SMILES: CC(C)NC(=O)[C@H]1CCC[C@H]1Nc1nc(Nc2ccc(C(=O)NC3CCN(C)CC3)c(F)c2)ncc1C(F)(F)F] | 0.19 | DCM:MeOH: NH₃ 9:1:0.1 | | 1.53 | 566 | 305 |
| 75 | 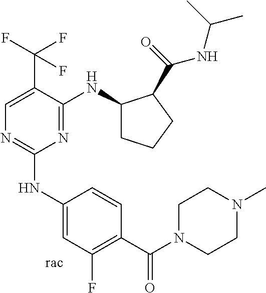 [SMILES: CC(C)NC(=O)[C@H]1CCC[C@H]1Nc1nc(Nc2ccc(C(=O)N3CCN(C)CC3)c(F)c2)ncc1C(F)(F)F] | 0.38 | DCM:MeOH: NH₃ 9:1:0.1 | | 1.46 | 552 | 299 |
| 76 | 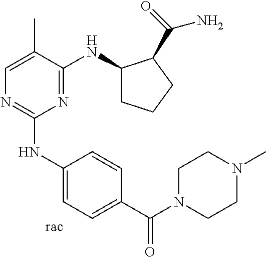 [SMILES: Cc1cnc(Nc2ccc(C(=O)N3CCN(C)CC3)cc2)nc1N[C@@H]1CCC[C@@H]1C(N)=O] | 0.33 | DCM:MeOH: NH₃ 5:1:0.1 | | | 438 | |
| 77 | 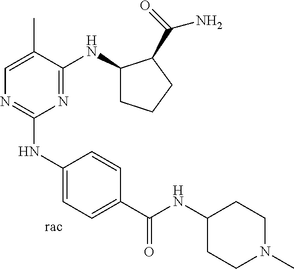 [SMILES: Cc1cnc(Nc2ccc(C(=O)NC3CCN(C)CC3)cc2)nc1N[C@@H]1CCC[C@@H]1C(N)=O] | 0.12 | DCM:MeOH: NH₃ 5:1:0.1 | | | 452 | |
| 78 | 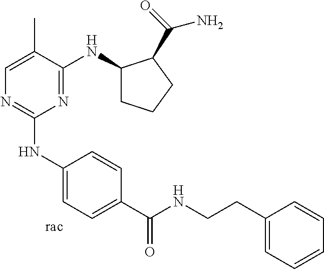 [SMILES: Cc1cnc(Nc2ccc(C(=O)NCCc3ccccc3)cc2)nc1N[C@@H]1CCC[C@@H]1C(N)=O] | 0.34 | DCM:MeOH: NH₃ 5:1:0.1 | | 1.53 | 459 | 284 |
| 79 | 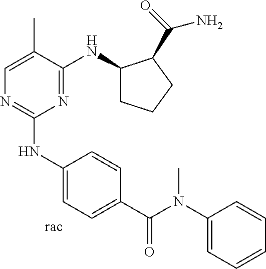 [SMILES: Cc1cnc(Nc2ccc(C(=O)N(C)c3ccccc3)cc2)nc1N[C@@H]1CCC[C@@H]1C(N)=O] | 0.69 | DCM:MeOH: NH₃ 5:1:0.1 | | 1.40 | 445 | 258/284 |
| 80 | 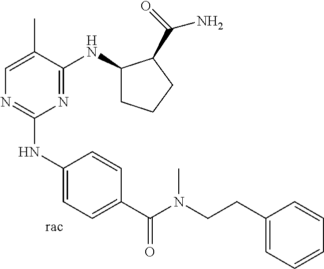 [SMILES: Cc1cnc(Nc2ccc(C(=O)N(C)CCc3ccccc3)cc2)nc1N[C@@H]1CCC[C@@H]1C(N)=O] | 0.75 | DCM:MeOH: NH₃ 5:1:0.1 | 213- 214 | 1.53 | 473 | 266 |
| 81 | 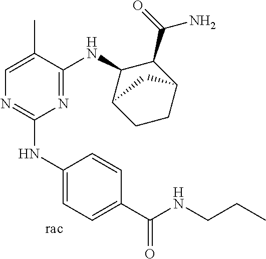 [SMILES: CCCNC(=O)c1ccc(Nc2ncc(C)c(N[C@@H]3[C@@H]4CC[C@@H](C4)[C@@H]3C(N)=O)n2)cc1] | 0.39 | EE:MeOH 8:2 | 228- 231 | 1.61 | 421 | 283 |
| 82 | 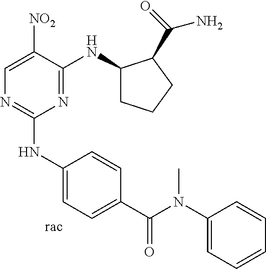 [SMILES: CN(C(=O)c1ccc(Nc2ncc([N+](=O)[O-])c(N[C@@H]3CCC[C@@H]3C(N)=O)n2)cc1)c1ccccc1] | 0.65 | DCM:MeOH: NH₃ 5:1:0.1 | 239- 242 | 1.95 | 476 | 262/384 |
| 83 | 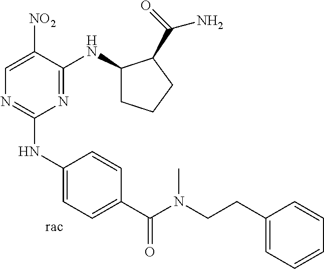 [SMILES: CN(CCc1ccccc1)C(=O)c1ccc(Nc2ncc([N+](=O)[O-])c(N[C@@H]3CCC[C@@H]3C(N)=O)n2)cc1] | 0.74 | DCM:MeOH: NH₃ 5:1:0.1 | 132- 134 | 2.06 | 504 | 258/383 |
| 84 | 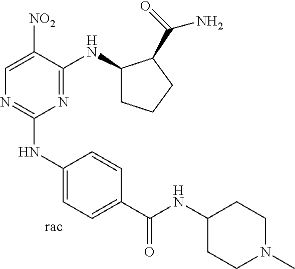 [SMILES: CN1CCC(NC(=O)c2ccc(Nc3ncc([N+](=O)[O-])c(N[C@@H]4CCC[C@@H]4C(N)=O)n3)cc2)CC1] | 0.16 | DCM:MeOH: NH₃ 5:1:0.1 | | 1.41 | 483 | 268/384 |
| 85 | 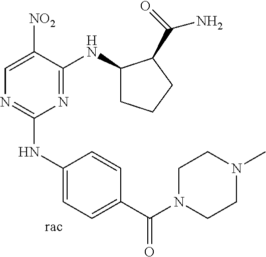 [SMILES: CN1CCN(C(=O)c2ccc(Nc3ncc([N+](=O)[O-])c(N[C@@H]4CCC[C@@H]4C(N)=O)n3)cc2)CC1] | 0.47 | DCM:MeOH: NH₃ 5:1:0.1 | 222- 224 | 1.39 | 469 | 262/383 |
| 86 | 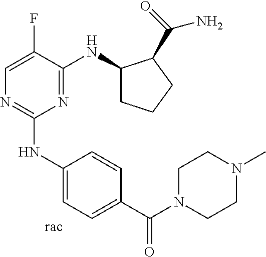 [SMILES: CN1CCN(C(=O)c2ccc(Nc3ncc(F)c(N[C@@H]4CCC[C@@H]4C(N)=O)n3)cc2)CC1] | 0.35 | DCM:MeOH: NH₃ 5:1:0.1 | | | 442 | |
| 87 | 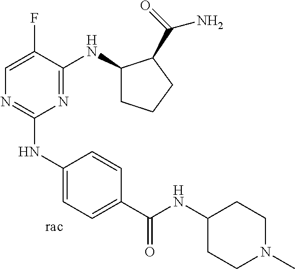 [SMILES: CN1CCC(NC(=O)c2ccc(Nc3ncc(F)c(N[C@@H]4CCC[C@@H]4C(N)=O)n3)cc2)CC1] | 0.18 | DCM:MeOH: NH₃ 5:1:0.1 | | | 456 | |
| 88 | 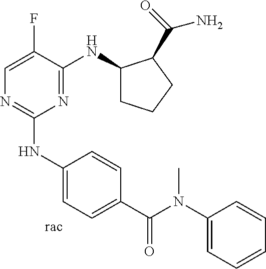 [SMILES: CN(C(=O)c1ccc(Nc2ncc(F)c(N[C@@H]3CCC[C@@H]3C(N)=O)n2)cc1)c1ccccc1] | 0.75 | DCM:MeOH: NH₃ 5:1:0.1 | | 1.52 | 449 | 261 |
| 89 | 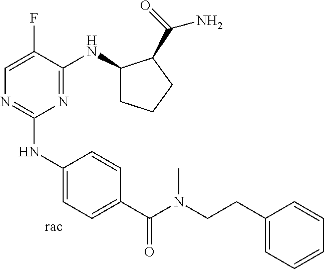 [SMILES: CN(CCc1ccccc1)C(=O)c1ccc(Nc2ncc(F)c(N[C@@H]3CCC[C@@H]3C(N)=O)n2)cc1] | 0.74 | DCM:MeOH: NH₃ 5:1:0.1 | | 1.62 | 477 | 265 |
| 90 | 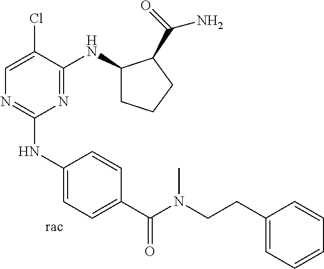 [SMILES: CN(CCc1ccccc1)C(=O)c1ccc(Nc2ncc(Cl)c(N[C@@H]3CCC[C@@H]3C(N)=O)n2)cc1] | 0.72 | DCM:MeOH: NH₃ 5:1:0.1 | | 1.79 | 493 | 275 |
| 91 | 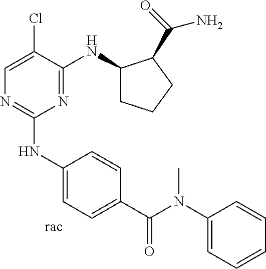 [SMILES: CN(C(=O)c1ccc(Nc2ncc(Cl)c(N[C@@H]3CCC[C@@H]3C(N)=O)n2)cc1)c1ccccc1] | 0.78 | DCM:MeOH: NH₃ 5:1:0.1 | | 1.67 | 465 | 286 |
| 92 | 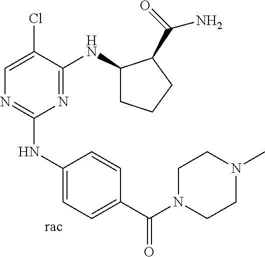 [SMILES: CN1CCN(C(=O)c2ccc(Nc3ncc(Cl)c(N[C@@H]4CCC[C@@H]4C(N)=O)n3)cc2)CC1] | 0.70 | DCM:MeOH: NH₃ 5:1:0.1 | | | 458 | |
| 93 | 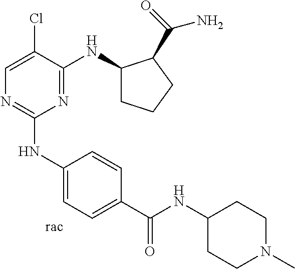 [SMILES: CN1CCC(NC(=O)c2ccc(Nc3ncc(Cl)c(N[C@@H]4CCC[C@@H]4C(N)=O)n3)cc2)CC1] | 0.18 | DCM:MeOH: NH₃ 5:1:0.1 | | 1.18 | 472 | 285 |
| 94 | 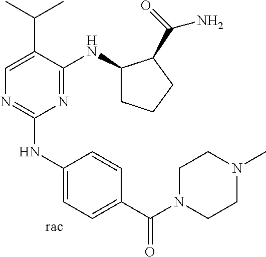 [SMILES: CC(C)c1cnc(Nc2ccc(C(=O)N3CCN(C)CC3)cc2)nc1N[C@@H]1CCC[C@@H]1C(N)=O] | 0.51 | DCM:MeOH: NH₃ 5:1:0.1 | | 1.15 | 466 | 273 |
| 95 | 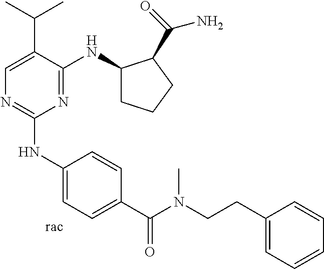 [SMILES: CC(C)c1cnc(Nc2ccc(C(=O)N(C)CCc3ccccc3)cc2)nc1N[C@@H]1CCC[C@@H]1C(N)=O] | 0.84 | DCM:MeOH: NH₃ 5:1:0.1 | | 1.64 | 501 | 267 |
| 96 | 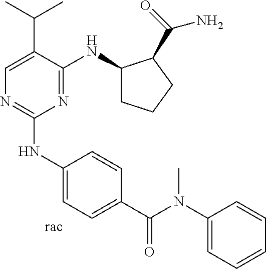 [SMILES: CC(C)c1cnc(Nc2ccc(C(=O)N(C)c3ccccc3)cc2)nc1N[C@@H]1CCC[C@@H]1C(N)=O] | 0.72 | DCM:MeOH: NH₃ 5:1:0.1 | 226- 229 | 1.51 | 473 | 262/283 |
| 97 | 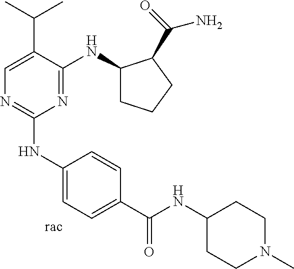 [SMILES: CC(C)c1cnc(Nc2ccc(C(=O)NC3CCN(C)CC3)cc2)nc1N[C@@H]1CCC[C@@H]1C(N)=O] | 0.17 | DCM:MeOH: NH₃ 5:1:0.1 | | 1.20 | 480 | 285 |
| 98 | 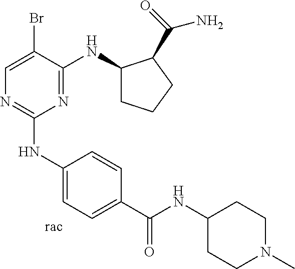 [SMILES: CN1CCC(NC(=O)c2ccc(Nc3ncc(Br)c(N[C@@H]4CCC[C@@H]4C(N)=O)n3)cc2)CC1] | 0.23 | DCM:MeOH: NH₃ 5:1:0.1 | 172- 174 | 1.24 | 516/518 | 286 |
| 99 | 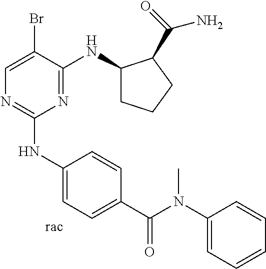 [SMILES: CN(C(=O)c1ccc(Nc2ncc(Br)c(N[C@@H]3CCC[C@@H]3C(N)=O)n2)cc1)c1ccccc1] | 0.60 | DCM:MeOH: NH₃ 5:1:0.1 | 144- 146 | 1.72 | 509/511 | 286 |
| 100 | 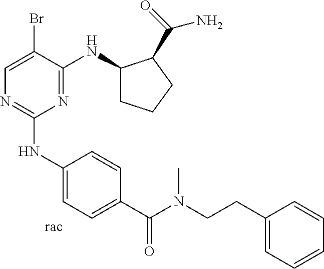 [SMILES: CN(CCc1ccccc1)C(=O)c1ccc(Nc2ncc(Br)c(N[C@@H]3CCC[C@@H]3C(N)=O)n2)cc1] | 0.53 | DCM:MeOH: NH₃ 5:1:0.1 | | 1.81 | 537/539 | 276 |
| 101 | 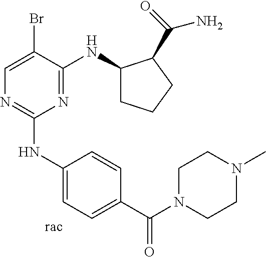 [SMILES: CN1CCN(C(=O)c2ccc(Nc3ncc(Br)c(N[C@@H]4CCC[C@@H]4C(N)=O)n3)cc2)CC1] | 0.45 | DCM:MeOH: NH₃ 5:1:0.1 | | | 502/504 | |
| 102 | 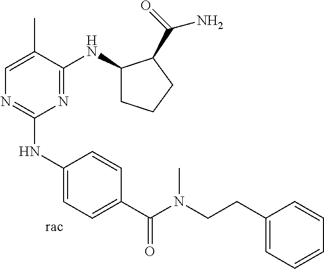 [SMILES: Cc1cnc(Nc2ccc(C(=O)N(C)CCc3ccccc3)cc2)nc1N[C@@H]1CCC[C@@H]1C(N)=O] | 0.61 | DCM:MeOH: NH₃ 5:1:0.1 | | 1.77 | 585 | 277 |
| 103 | 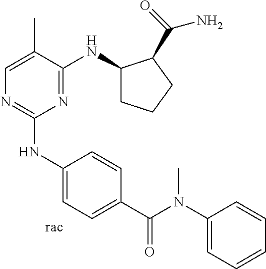 [SMILES: Cc1cnc(Nc2ccc(C(=O)N(C)c3ccccc3)cc2)nc1N[C@@H]1CCC[C@@H]1C(N)=O] | 0.68 | DCM:MeOH: NH₃ 5:1:0.1 | 145- 148 | 1.68 | 557 | 288 |
| 104 | 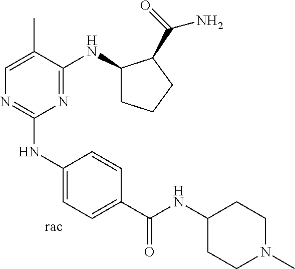 [SMILES: Cc1cnc(Nc2ccc(C(=O)NC3CCN(C)CC3)cc2)nc1N[C@@H]1CCC[C@@H]1C(N)=O] | 0.25 | DCM:MeOH: NH₃ 5:1:0.1 | 171- 174 | 1.27 | 564 | 290 |
| 105 | 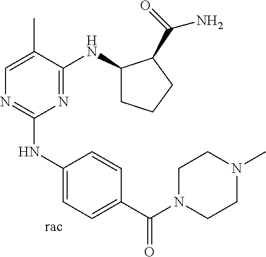 [SMILES: Cc1cnc(Nc2ccc(C(=O)N3CCN(C)CC3)cc2)nc1N[C@@H]1CCC[C@@H]1C(N)=O] | 0.58 | DCM:MeOH: NH₃ 5:1:0.1 | | 1.15 | 550 | 278 |
| 106 | 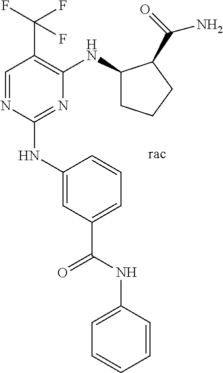 [SMILES: NC(=O)[C@H]1CCC[C@H]1Nc1nc(Nc2cccc(C(=O)Nc3ccccc3)c2)ncc1C(F)(F)F] | 0.53 | EE:cHex | | 1.96 | 485 | 268 |
| 107 | 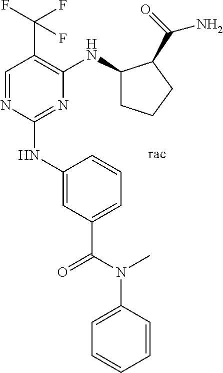 [SMILES: CN(C(=O)c1cccc(Nc2ncc(C(F)(F)F)c(N[C@@H]3CCC[C@@H]3C(N)=O)n2)c1)c1ccccc1] | 0.35 | EE | | 1.95 | 499 | 266 |
| 108 | 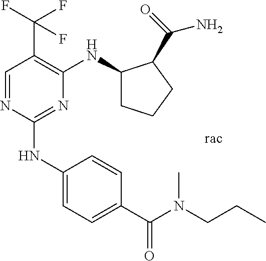 [SMILES: CCCN(C)C(=O)c1ccc(Nc2ncc(C(F)(F)F)c(N[C@@H]3CCC[C@@H]3C(N)=O)n2)cc1] | 0.16 | EE | | 1.86 | 465 | 275 |
| 109 | 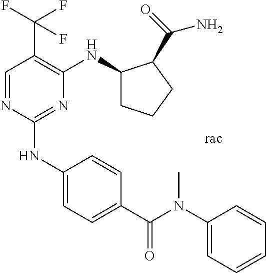 [SMILES: CN(C(=O)c1ccc(Nc2ncc(C(F)(F)F)c(N[C@@H]3CCC[C@@H]3C(N)=O)n2)cc1)c1ccccc1] | 0.30 | EE | | 1.94 | 499 | 299 |
| 110 | 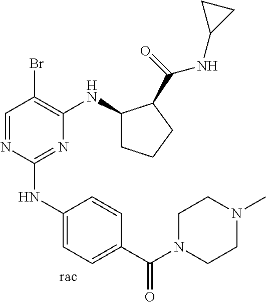 [SMILES: CN1CCN(C(=O)c2ccc(Nc3ncc(Br)c(N[C@@H]4CCC[C@@H]4C(=O)NC4CC4)n3)cc2)CC1] | 0.62 | DCM:MeOH: NH₃ 5:1:0.1 | | 1.33 | 542/544 | 279 |
| 111 | 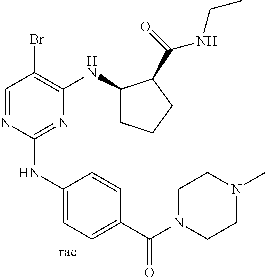 [SMILES: CCNC(=O)[C@H]1CCC[C@H]1Nc1nc(Nc2ccc(C(=O)N3CCN(C)CC3)cc2)ncc1Br] | 0.64 | DCM:MeOH: NH₃ 5:1:0.1 | | 1.30 | 530 | 278 |
| 112 | 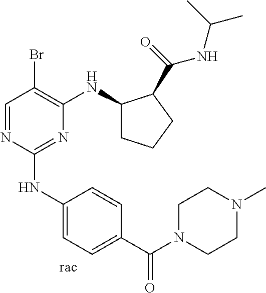 [SMILES: CC(C)NC(=O)[C@H]1CCC[C@H]1Nc1nc(Nc2ccc(C(=O)N3CCN(C)CC3)cc2)ncc1Br] | 0.68 | DCM:MeOH: NH₃ 5:1:0.1 | | 1.39 | 544/546 | 280 |
| 113 | 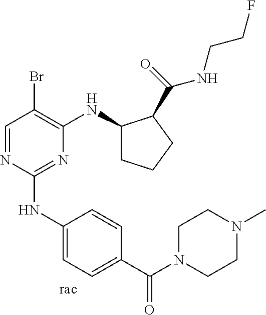 [SMILES: CN1CCN(C(=O)c2ccc(Nc3ncc(Br)c(N[C@@H]4CCC[C@@H]4C(=O)NCCF)n3)cc2)CC1] | 0.67 | DCM:MeOH: NH₃ 5:1:0.1 | | 1.26 | 548/550 | 278 |
| 114 | 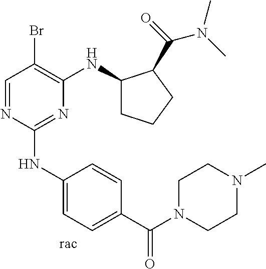 [SMILES: CN1CCN(C(=O)c2ccc(Nc3ncc(Br)c(N[C@@H]4CCC[C@@H]4C(=O)N(C)C)n3)cc2)CC1] | 0.64 | DCM:MeOH: NH₃ 5:1:0.1 | | 1.29 | 530/532 | 279 |
| 115 | 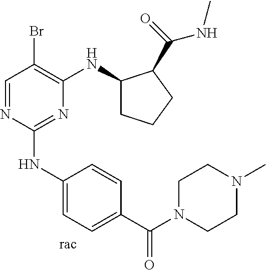 [SMILES: CNC(=O)[C@H]1CCC[C@H]1Nc1nc(Nc2ccc(C(=O)N3CCN(C)CC3)cc2)ncc1Br] | 0.55 | DCM:MeOH: NH₃ 5:1:0.1 | | | 516/518 | |
| 116 | 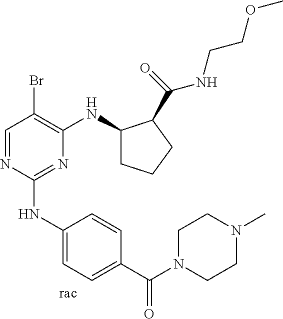 [SMILES: COCCNC(=O)[C@H]1CCC[C@H]1Nc1nc(Nc2ccc(C(=O)N3CCN(C)CC3)cc2)ncc1Br] | 0.61 | DCM:MeOH: NH₃ 5:1:0.1 | | 1.25 | 560/562 | 279 |
| 117 | 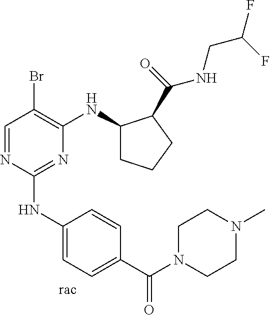 [SMILES: CN1CCN(C(=O)c2ccc(Nc3ncc(Br)c(N[C@@H]4CCC[C@@H]4C(=O)NCC(F)F)n3)cc2)CC1] | 0.66 | DCM:MeOH: NH₃ 5:1:0.1 | | 1.34 | 566/568 | 279 |
| 118 | 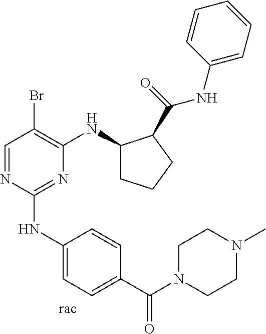 [SMILES: CN1CCN(C(=O)c2ccc(Nc3ncc(Br)c(N[C@@H]4CCC[C@@H]4C(=O)Nc4ccccc4)n3)cc2)CC1] | 0.73 | DCM:MeOH: NH₃ 5:1:0.1 | | 1.36 | 579/581 | 277 |
| 119 | 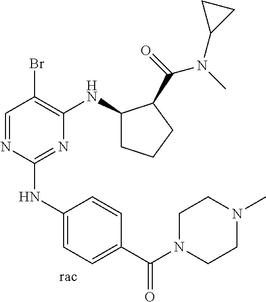 [SMILES: CN1CCN(C(=O)c2ccc(Nc3ncc(Br)c(N[C@@H]4CCC[C@@H]4C(=O)N(C)C4CC4)n3)cc2)CC1] | 0.71 | DCM:MeOH: NH₃ 5:1:0.1 | | 1.45 | 556/558 | 280 |
| 120 | 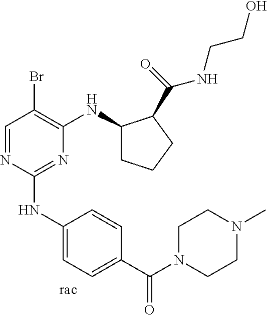 [SMILES: CN1CCN(C(=O)c2ccc(Nc3ncc(Br)c(N[C@@H]4CCC[C@@H]4C(=O)NCCO)n3)cc2)CC1] | 0.44 | DCM:MeOH: NH₃ 5:1:0.1 | | | 546/548 | |
| 121 | 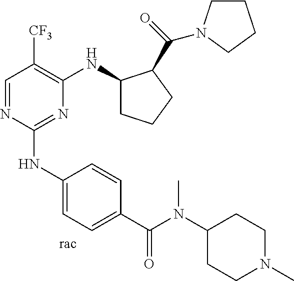 [SMILES: CN1CCC(N(C)C(=O)c2ccc(Nc3ncc(C(F)(F)F)c(N[C@@H]4CCC[C@@H]4C(=O)N4CCCC4)n3)cc2)CC1] | 0.47 | DCM:MeOH: NH₃ 5:1:0.1 | | | 574 | |
| 122 | 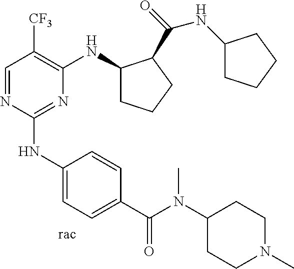 [SMILES: CN1CCC(N(C)C(=O)c2ccc(Nc3ncc(C(F)(F)F)c(N[C@@H]4CCC[C@@H]4C(=O)NC4CCCC4)n3)cc2)CC1] | 0.56 | DCM:MeOH: NH₃ 5:1:0.1 | | | 588 | |
| 123 | 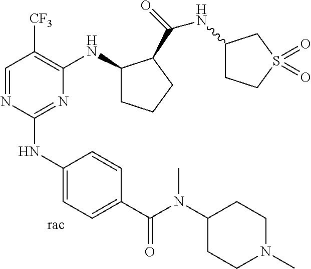 [SMILES: CN1CCC(N(C)C(=O)c2ccc(Nc3ncc(C(F)(F)F)c(N[C@@H]4CCC[C@@H]4C(=O)NC4CCS(=O)(=O)C4)n3)cc2)CC1] | 0.17 | DCM:MeOH: NH₃ 5:1:0.1 | | | 638 | 278 |
| 124 | 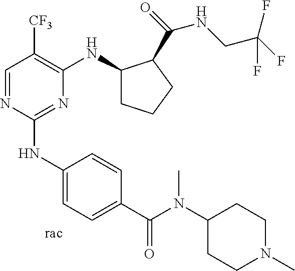 [SMILES: CN1CCC(N(C)C(=O)c2ccc(Nc3ncc(C(F)(F)F)c(N[C@@H]4CCC[C@@H]4C(=O)NCC(F)(F)F)n3)cc2)CC1] | 0.19 | DCM:MeOH: NH₃ 5:1:0.1 | 179- 184x | | 602 | 278 |
| 125 | 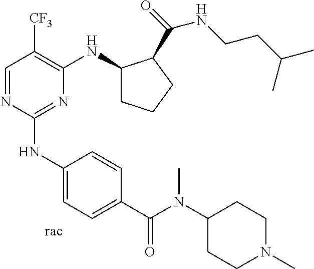 [SMILES: CC(C)CCNC(=O)[C@H]1CCC[C@H]1Nc1nc(Nc2ccc(C(=O)N(C)C3CCN(C)CC3)cc2)ncc1C(F)(F)F] | 0.25 | DCM:MeOH: NH₃ 5:1:0.1 | 129- 134 | | 590 | 246/278 |
| 126 | 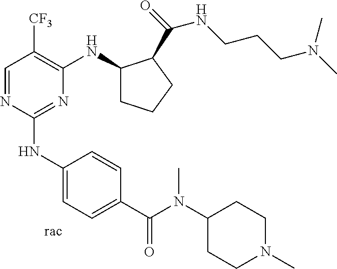 [SMILES: CN(C)CCCNC(=O)[C@H]1CCC[C@H]1Nc1nc(Nc2ccc(C(=O)N(C)C3CCN(C)CC3)cc2)ncc1C(F)(F)F] | 0.03 | DCM:MeOH: NH₃ 5:1:0.1 | | | 605 | 246/278 |
| 127 | 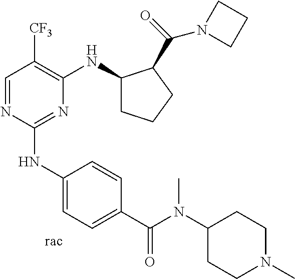 [SMILES: CN1CCC(N(C)C(=O)c2ccc(Nc3ncc(C(F)(F)F)c(N[C@@H]4CCC[C@@H]4C(=O)N4CCC4)n3)cc2)CC1] | 0.24 | DCM:MeOH: NH₃ 5:1:0.1 | | | 560 | 274 |
| 128 | 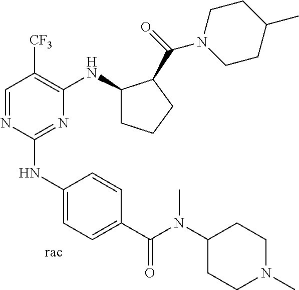 [SMILES: CC1CCN(C(=O)[C@H]2CCC[C@H]2Nc2nc(Nc3ccc(C(=O)N(C)C4CCN(C)CC4)cc3)ncc2C(F)(F)F)CC1] | 0.74 | DCM:MeOH: NH₃ 5:1:0.1 | | | 602 | |
| 129 | 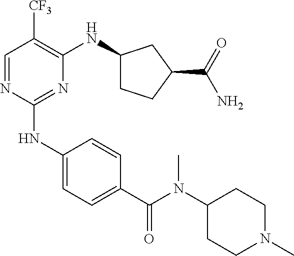 [SMILES: CN1CCC(N(C)C(=O)c2ccc(Nc3ncc(C(F)(F)F)c(N[C@@H]4CC[C@H](C(N)=O)C4)n3)cc2)CC1] | 0.10 | DCM:MeOH: NH₃ 5:1:0.1 | | | 520 | 270 |
| 130 | 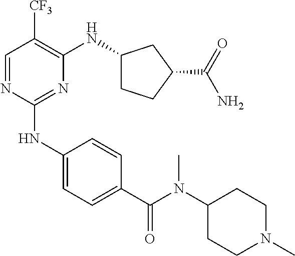 [SMILES: CN1CCC(N(C)C(=O)c2ccc(Nc3ncc(C(F)(F)F)c(N[C@H]4CC[C@@H](C(N)=O)C4)n3)cc2)CC1] | 0.10 | DCM:MeOH: NH₃ 5:1:0.1 | 211 (de- comp.) | | 520 | 270 |
| 131 | 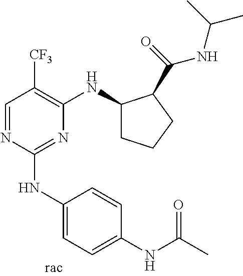 [SMILES: CC(=O)Nc1ccc(Nc2ncc(C(F)(F)F)c(N[C@@H]3CCC[C@@H]3C(=O)NC(C)C)n2)cc1] | 0.70 | DCM:MeOH: NH₃ 5:1:0.1 | | | 465 | 278 |
| 132 | 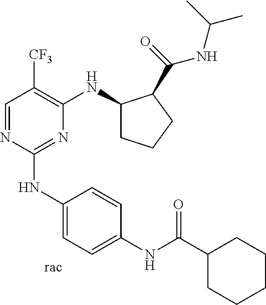 [SMILES: CC(C)NC(=O)[C@H]1CCC[C@H]1Nc1nc(Nc2ccc(NC(=O)C3CCCCC3)cc2)ncc1C(F)(F)F] | | DCM:MeOH: NH₃ 5:1:0.1 | 0.83 | | 533 | 242/282 |
| 133 | 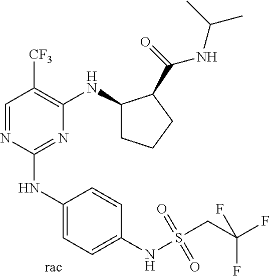 [SMILES: CC(C)NC(=O)[C@H]1CCC[C@H]1Nc1nc(Nc2ccc(NS(=O)(=O)CC(F)(F)F)cc2)ncc1C(F)(F)F] | 0.62 | DCM:MeOH: NH₃ 5:1:0.1 | | | 569 | 274 |
| 134 | 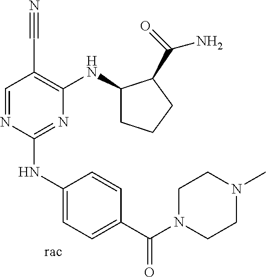 [SMILES: CN1CCN(C(=O)c2ccc(Nc3ncc(C#N)c(N[C@@H]4CCC[C@@H]4C(N)=O)n3)cc2)CC1] | 0.33 | DCM:MeOH: NH₃ 5:1:0.1 | | 1.28 | 449 | 319 |
| 135 | 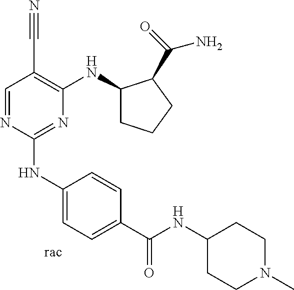 [SMILES: CN1CCC(NC(=O)c2ccc(Nc3ncc(C#N)c(N[C@@H]4CCC[C@@H]4C(N)=O)n3)cc2)CC1] | 0.08 | DCM:MeOH: NH₃ 5:1:0.1 | | 1.37 | 463 | 322 |
| 136 | 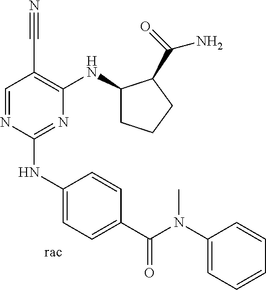 [SMILES: CN(C(=O)c1ccc(Nc2ncc(C#N)c(N[C@@H]3CCC[C@@H]3C(N)=O)n2)cc1)c1ccccc1] | 0.68 | DCM:MeOH: NH₃ 5:1:0.1 | | 1.91 | 456 | 323 |
| 137 | 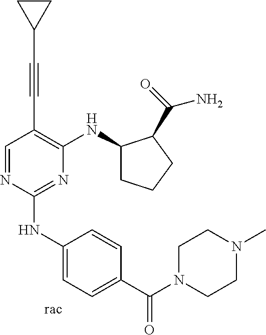 [SMILES: CN1CCN(C(=O)c2ccc(Nc3ncc(C#CC4CC4)c(N[C@@H]4CCC[C@@H]4C(N)=O)n3)cc2)CC1] | 0.37 | DCM:MeOH: NH₃ 9:1:0.1 | 151- 154 | 1.35 | 488 | 297 |
| 138 | 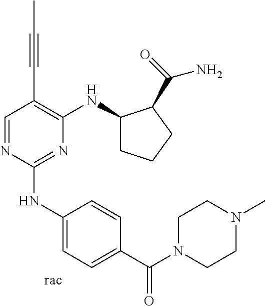 [SMILES: CC#Cc1cnc(Nc2ccc(C(=O)N3CCN(C)CC3)cc2)nc1N[C@@H]1CCC[C@@H]1C(N)=O] | 0.32 | DCM:MeOH: NH₃ 9:1:0.1 | 167- 169 | 1.15 | 462 | 291 |
| 139 | 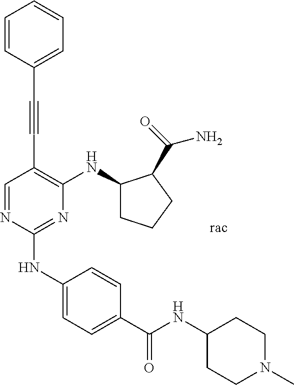 [SMILES: CN1CCC(NC(=O)c2ccc(Nc3ncc(C#Cc4ccccc4)c(N[C@@H]4CCC[C@@H]4C(N)=O)n3)cc2)CC1] | 0.19 | DCM:MeOH: NH₃ 5:1:0.1 | 156- 158 | | 538 | |
| 140 | 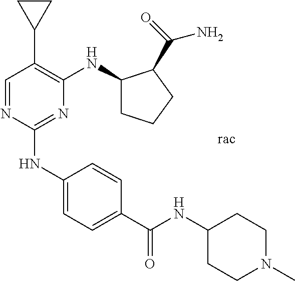 [SMILES: CN1CCC(NC(=O)c2ccc(Nc3ncc(C4CC4)c(N[C@@H]4CCC[C@@H]4C(N)=O)n3)cc2)CC1] | | | | 1.20 | 478 | 286 |
| 141 | 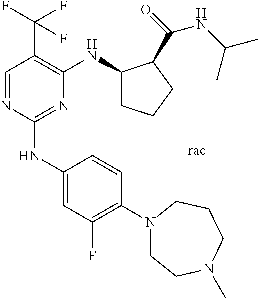 [SMILES: CC(C)NC(=O)[C@H]1CCC[C@H]1Nc1nc(Nc2ccc(N3CCCN(C)CC3)c(F)c2)ncc1C(F)(F)F] | 0.16 | DCM:MeOH: NH₃ 9:1:0.1 | | 1.38 | 538 | 280 |
| 142 | 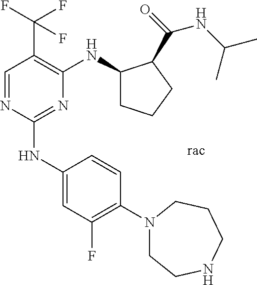 [SMILES: CC(C)NC(=O)[C@H]1CCC[C@H]1Nc1nc(Nc2ccc(N3CCCNCC3)c(F)c2)ncc1C(F)(F)F] | 0.65 | DCM:MeOH: NH₃ 9:1:0.1 | | 1.52 | 524 | 280 |
| 143 | 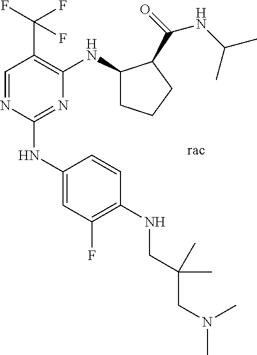 [SMILES: CC(C)NC(=O)[C@H]1CCC[C@H]1Nc1nc(Nc2ccc(NCC(C)(C)CN(C)C)c(F)c2)ncc1C(F)(F)F] | 0.66 | DCM:MeOH: NH₃ 9:1:0.1 | | 1.51 | 554 | 275 |
| 144 | 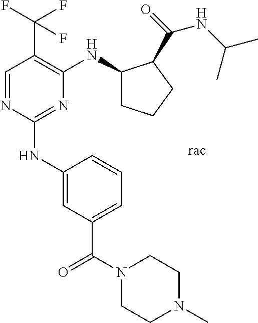 [SMILES: CC(C)NC(=O)[C@H]1CCC[C@H]1Nc1nc(Nc2cccc(C(=O)N3CCN(C)CC3)c2)ncc1C(F)(F)F] | 0.58 | DCM:MeOH: NH₃ 9:1:0.1 | | 1.57 | 534 | 268 |
| 145 | 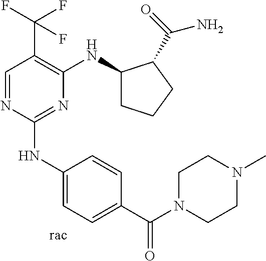 [SMILES: CN1CCN(C(=O)c2ccc(Nc3ncc(C(F)(F)F)c(N[C@@H]4CCC[C@H]4C(N)=O)n3)cc2)CC1] | 0.47 | DCM:MeOH: NH₃ 5:1:0.1 | | 1.24 | 492 | 276 |
| 146 | 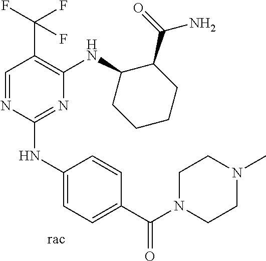 [SMILES: CN1CCN(C(=O)c2ccc(Nc3ncc(C(F)(F)F)c(N[C@@H]4CCCC[C@@H]4C(N)=O)n3)cc2)CC1] | 0.61 | DCM:MeOH: NH₃ 5:1:0.1 | | 1.43 | 506 | 277 |
| 147 | 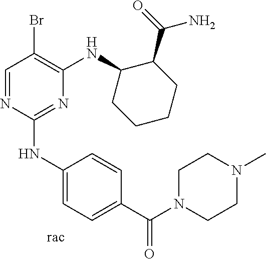 [SMILES: CN1CCN(C(=O)c2ccc(Nc3ncc(Br)c(N[C@@H]4CCCC[C@@H]4C(N)=O)n3)cc2)CC1] | 0.58 | DCM:MeOH: NH₃ 5:1:0.1 | | 1.21 | 516/518 | 278 |
| 148 | 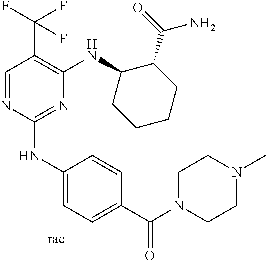 [SMILES: CN1CCN(C(=O)c2ccc(Nc3ncc(C(F)(F)F)c(N[C@@H]4CCCC[C@H]4C(N)=O)n3)cc2)CC1] | 0.55 | DCM:MeOH: NH₃ 5:1:0.1 | | 1.22 | 506 | 275 |

The Examples describe the biological activity of the compounds according to the invention without restricting the invention to these Examples.

As demonstrated by DNA staining followed by FACS or Cellomics Array Scan analysis, the inhibition of proliferation brought about by the compounds according to the invention is mediated above all by errors in chromosome segregation. Because of the accumulation of faulty segregations, massive polyploidia occurs which may finally lead to inhibition of proliferation or even apoptosis. On the basis of their biological properties the compounds of general formula (I) according to the invention, their isomers and the physiologically acceptable salts thereof are suitable for treating diseases characterised by excessive or anomalous cell proliferation.

### Example Aurora-B Kinase Assay

A radioactive enzyme inhibition assay was developed using Baculovirus-expressed recombinant human Aurora B wild-type protein equipped at the N-terminal position with a histidine (6) epitope (His-), which is obtained from infected insect cells (SF21) and purified.

### Expression and Purification

For this, 300×10⁶ SF21 cells in SF-900II insect cell medium (Invitrogen) are incubated for example with a suitable amount of Baculovirus solution for 1 h at 27° C. (Fernbach flask agitator, 50 rpm). Then 250 ml SF-900 II medium is added and agitated for 3 days (100 rpm, 27° C.). Three hours before harvesting, okadaic acid (C₄₄H₆₈O₁₃, Calbiochem #495604) is added (final concentration 0.1 μM) in order to stabilise phosphorylation sites on recombinant Aurora B. The cells are pelleted by centrifugation (1000 rpm, 5 min, 4° C.), the supernatant is discarded and the pellet is frozen in liquid nitrogen. The pellet is thawed (37° C., 5 min) and resuspended in lysing buffer. 40 mL lysing buffer (25 mM Tris/Cl, 10 mM MgCl₂, 300 mM NaCl, 20 mM imidazole, pH 8.0, 0.07% 2-mercaptoethanol and Protease-Inhibitor-Complete from Roche Diagnostics) is used for 200 mL of volume of the starting culture. After two rapid freezing/thawing cycles (liquid nitrogen at 37° C.), the lysate is kept on ice for 30 min, then incubated (2 h, 4° C.) with washed Ni-NTA beads (Ni-NTA Superflow Beads, 4 mL per 200 mL of starting culture) and placed in an Econo-Pac column (Biorad #732-1010). Five washes with in each case 10 column volumes of washing buffer (25 mM Tris/Cl, 10 mM MgCl₂, 1000 mM NaCl, 20 mM imidazole, pH 8.0, 0.07% 2-mercaptoethanol and Protease-Inhibitor-Complete from Roche Diagnostics) precede the elution in 8 ml (per 200 ml of starting culture) elution buffer (25 mM Tris/Cl pH 8.0, 300 mM NaCl, 10 mM MgCl₂, 0.03% Brij-35, 10% glycerol, 0.07% 2-mercaptoethanol, 400 mM imidazole). The combined eluate fractions are desalinated using a Sephadex G25 column and transferred into freezing buffer (50 mM tris/Cl pH 8.0, 150 mM NaCl, 0.1 mM EDTA, 0.03% Brij-35, 10% glycerol, 1 mM DTT).

### Kinase Assay

Test substances are placed in a polypropylene dish (96 wells, Greiner #655 201), in order to cover a concentration frame of 10 μM-0.0001 μM. The final concentration of DMSO in the assay is 5%. 30 μL of protein mix (50 mM tris/Cl pH 7.5, 25 mM MgCl₂, 25 mM NaCl, 167 μM ATP, 200 ng His-Aurora B in freezing buffer) are pipetted into the 10 μl of test substance provided in 25% DMSO and this is incubated for 15 min at RT. Then 10 μL of peptide mix (100 mM tris/Cl pH 7.5, 50 mM MgCl₂, 50 mM NaCl, 5 μM NaF, 5 μM DTT, 1 μCi gamma-P33-ATP [Amersham], 50 μM substrate peptide [biotin-EPLERRLSLVPDS or multimers thereof, or biotin-EPLERRLSLVPKM or multimers thereof, or biotin-LRRWSLGLRRWSLGLRRWSLGLRRWSLG]) are added. The reaction is incubated for 75 min (ambient temperature) and stopped by the addition of 180 μL of 6.4% trichloroacetic acid and incubated for 20 min on ice. A multiscreen filtration plate (Millipore, MAIP NOB 10) is equilibrated first of all with 100 μL 70% ethanol and then with 180 μL trichloroacetic acid and the liquids are eliminated using a suitable suction apparatus. Then the stopped kinase reaction is applied. After 5 washing steps with 180 μL 1% trichloroacetic acid in each case the lower half of the dish is dried (10-20 min at 55° C.) and 25 μL scintillation cocktail (Microscint, Packard #6013611) is added. Incorporated gamma-phosphate is quantified using a Wallac 1450 Microbeta Liquid Scintillation Counter. Samples without test substance or without substrate peptide are used as controls. IC₅₀ values are obtained using Graph Pad Prism software.

The anti-proliferative activity of the compounds according to the invention is determined in the proliferation test on cultivated human tumour cells and/or in a cell cycle analysis, for example on NCI-H460 tumour cells. In both test methods the compounds exhibit good to very good activity, i.e. for example an EC50 value in the NCI-H460 proliferation test of less than 5 μmol/L, generally less than 1 μmol/L.

### Measurement of the Inhibition of Proliferation on Cultivated Human Tumour Cells

To measure proliferation on cultivated human tumour cells, cells of lung tumour cell line NCI-H460 (obtained from American Type Culture Collection (ATCC)) are cultivated in RPMI 1640 medium (Gibco) and 10% foetal calf serum (Gibco) and harvested in the log growth phase. Then the NCI-H460 cells are placed in 96-well flat-bottomed plates (Falcon) at a density of 1000 cells per well in RPMI 1640 medium and incubated overnight in an incubator (at 37° C. and 5% CO₂). The active substances are added to the cells in various concentrations (dissolved in DMSO; DMSO final concentration: 0.1%). After 72 hours incubation 20 μl AlamarBlue reagent (AccuMed International) is added to each well, and the cells are incubated for a further 5-7 hours. After incubation the colour change of the AlamarBlue reagent is determined in a Wallac Microbeta fluorescence spectrophotometer. EC₅₀ values are calculated using Standard Levenburg Marquard algorithms (GraphPadPrizm). Cell cycle analyses are carried out for example using FACS analyses

(Fluorescence Activated Cell Sorter) or by Cellomics Array Scan (CellCycle Analysis).

### FACS Analysis

Propidium iodide (PI) binds stoichiometrically to double-stranded DNA, and is thus suitable for determining the proportion of cells in the G1, S, and G2/M phase of the cell cycle on the basis of the cellular DNA content. Cells in the G0 and G1 phase have a diploid DNA content (2N), whereas cells in the G2 or mitosis phase have a 4N DNA content.

For PI staining, for example, 0.4 million 1.75×10⁶ NCI-H460 cells are seeded onto a 75 cm² cell culture flask, and after 24 h either 0.1% DMSO is added as control or the substance is added in various concentrations (in 0.1% DMSO). The cells are incubated for 42 h with the substance or with DMSO. Then the cells are detached with trypsin and centrifuged. The cell pellet is washed with bufferend saline solution (PBS) and the cells are then fixed with 80% at −20° C. for at least 2 h. After another washing step with PBS the cells are permeabilised with Triton X-100 (Sigma; 0.25% in PBS) on ice for 5 min, and then incubated with a solution of propidium iodide (Sigma; 10 μg/ml) and RNAse (Serva; 1 mg/mLl) in the ratio 9:1 for at least 20 min in the dark. The DNA measurement is carried out in a Becton Dickinson FACS Analyzer, with an argon laser (500 mW, emission 488 nm); data are obtained and evaluated using the DNA Cell Quest Programme (BD).

### Cellomics Array Scan

NCI-H460 cells are seeded into 96-well flat-bottomed dishes (Falcon) in RPMI 1640 medium (Gibco) with 10% foetal calf serum (Gibco) in a density of 2000 cells per well and incubated overnight in an incubator (at 37° C. and 5% CO₂). The active substances are added to the cells in various concentrations (dissolved in DMSO; DMSO final concentration: 0.1%). After 42 h incubation the medium is medium suction filtered, the cells are fixed for 10 min with 4% formaldehyde solution and Triton X-100 (1:200 in PBS) at ambient temperature and simultaneously permeabilised, and then washed twice with a 0.3% BSA solution (Calbiochem). Then the DNA is stained by the addition of 50 μL/well of 4′,6-diamidino-2-phenylindole (DAPI; Molecular Probes) in a final concentration of 300 nM for 1 h at ambient temperature, in the dark. The preparations are then carefully washed twice with PBS, the plates are stuck down with black adhesive film and analysed in the Cellomics ArrayScan using the CellCycle BioApplication programme and visualised and evaluated using Spotfire.

The substances of the present invention are Aurora kinase inhibitors. On the basis of their biological properties the compounds of general formula (1) according to the invention, their isomers and the physiologically acceptable salts thereof are suitable for treating diseases characterised by excessive or anomalous cell proliferation.

Such diseases include for example: viral infections (e.g. HIV and Kaposi's sarcoma); inflammatory and autoimmune diseases (e.g. colitis, arthritis, Alzheimer's disease, glomerulonephritis and wound healing); bacterial, fungal and/or parasitic infections; leukaemias, lymphomas and solid tumours (e.g. carcinomas and sarcomas), skin diseases (e.g. psoriasis); diseases based on hyperplasia which are characterised by an increase in the number of cells (e.g. fibroblasts, hepatocytes, bones and bone marrow cells, cartilage or smooth muscle cells or epithelial cells (e.g. endometrial hyperplasia)); bone diseases and cardiovascular diseases (e.g. restenosis and hypertrophy).

For example, the following cancers may be treated with compounds according to the invention, without being restricted thereto: brain tumours such as for example acoustic neurinoma, astrocytomas such as pilocytic astrocytomas, fibrillary astrocytoma, protoplasmic astrocytoma, gemistocytary astrocytoma, anaplastic astrocytoma and glioblastoma, brain lymphomas, brain metastases, hypophyseal tumour such as prolactinoma, HGH (human growth hormone) producing tumour and ACTH producing tumour (adrenocorticotropic hormone), craniopharyngiomas, medulloblastomas, meningeomas and oligodendrogliomas; nerve tumours (neoplasms) such as for example tumours of the vegetative nervous system such as neuroblastoma sympathicum, ganglioneuroma, paraganglioma (pheochromocytoma, chromaffinoma) and glomus-caroticum tumour, tumours on the peripheral nervous system such as amputation neuroma, neurofibroma, neurinoma (neurilemmoma, Schwannoma) and malignant Schwannoma, as well as tumours of the central nervous system such as brain and bone marrow tumours; intestinal cancer such as for example carcinoma of the rectum, colon, anus, small intestine and duodenum; eyelid tumours such as basalioma or basal cell carcinoma; pancreatic cancer or carcinoma of the pancreas; bladder cancer or carcinoma of the bladder; lung cancer (bronchial carcinoma) such as for example small-cell bronchial carcinomas (oat cell carcinomas) and non-small cell bronchial carcinomas such as plate epithelial carcinomas, adenocarcinomas and large-cell bronchial carcinomas; breast cancer such as for example mammary carcinoma such as infiltrating ductal carcinoma, colloid carcinoma, lobular invasive carcinoma, tubular carcinoma, adenocystic carcinoma and papillary carcinoma; non-Hodgkin's lymphomas (NHL) such as for example Burkitt's lymphoma, low-malignancy non-Hodgkin's lymphomas (NHL) and mucosis fungoides; uterine cancer or endometrial carcinoma or corpus carcinoma; CUP syndrome (Cancer of Unknown Primary); ovarian cancer or ovarian carcinoma such as mucinous, endometrial or serous cancer; gall bladder cancer; bile duct cancer such as for example Klatskin tumour; testicular cancer such as for example seminomas and non-seminomas; lymphoma (lymphosarcoma) such as for example malignant lymphoma, Hodgkin's disease, non-Hodgkin's lymphomas (NHL) such as chronic lymphatic leukaemia, leukaemic reticuloendotheliosis, immunocytoma, plasmocytoma (multiple myeloma), immunoblastoma, Burkitt's lymphoma, T-zone mycosis fungoides, large-cell anaplastic lymphoblastoma and lymphoblastoma; laryngeal cancer such as for example tumours of the vocal cords, supraglottal, glottal and subglottal laryngeal tumours; bone cancer such as for example osteochondroma, chondroma, chondroblastoma, chondromyxoid fibroma, osteoma, osteoid osteoma, osteoblastoma, eosinophilic granuloma, giant cell tumour, chondrosarcoma, osteosarcoma, Ewing's sarcoma, reticulo-sarcoma, plasmocytoma, giant cell tumour, fibrous dysplasia, juvenile bone cysts and aneurysmatic bone cysts; head and neck tumours such as for example tumours of the lips, tongue, floor of the mouth, oral cavity, gums, palate, salivary glands, throat, nasal cavity, paranasal sinuses, larynx and middle ear; liver cancer such as for example liver cell carcinoma or hepatocellular carcinoma (HCC); leukaemias, such as for example acute leukaemias such as acute lymphatic/lymphoblastic leukaemia (ALL), acute myeloid leukaemia (AML); chronic leukaemias such as chronic lymphatic leukaemia (CLL), chronic myeloid leukaemia (CML); stomach cancer or gastric carcinoma such as for example papillary, tubular and mucinous adenocarcinoma, signet ring cell carcinoma, adenosquamous carcinoma, small-cell carcinoma and undifferentiated carcinoma; melanomas such as for example superficially spreading, nodular, lentigo-maligna and acral-lentiginous melanoma; renal cancer such as for example kidney cell carcinoma or hypernephroma or Grawitz's tumour; oesophageal cancer or carcinoma of the oesophagus; penile cancer; prostate cancer; throat cancer or carcinomas of the pharynx such as for example nasopharynx carcinomas, oropharynx carcinomas and hypopharynx carcinomas; retinoblastoma such as for example vaginal cancer or vaginal carcinoma; plate epithelial carcinomas, adenocarcinomas, in situ carcinomas, malignant melanomas and sarcomas; thyroid carcinomas such as for example papillary, follicular and medullary thyroid carcinoma, as well as anaplastic carcinomas; spinalioma, epidormoid carcinoma and plate epithelial carcinoma of the skin; thymomas, cancer of the urethra and cancer of the vulva.

The new compounds may be used for the prevention, short-term or long-term treatment of the above-mentioned diseases, optionally also in combination with radiotherapy or other “state-of-the-art” compounds, such as e.g. cytostatic or cytotoxic substances, cell proliferation inhibitors, anti-angiogenic substances, steroids or antibodies.

The compounds of general formula (1) may be used on their own or in combination with other active substances according to the invention, optionally also in combination with other pharmacologically active substances.

Chemotherapeutic agents which may be administered in combination with the compounds according to the invention, include, without being restricted thereto, hormones, hormone analogues and antihormones (e.g. tamoxifen, toremifene, raloxifene, fulvestrant, megestrol acetate, flutamide, nilutamide, bicalutamide, aminoglutethimide, cyproterone acetate, finasteride, buserelin acetate, fludrocortinsone, fluoxymesterone, medroxyprogesterone, octreotide), aromatase inhibitors (e.g. anastrozole, letrozole, liarozole, vorozole, exemestane, atamestane), LHRH agonists and antagonists (e.g. goserelin acetate, luprolide), inhibitors of growth factors (growth factors such as for example “platelet derived growth factor” and “hepatocyte growth factor”, inhibitors are for example “growth factor” antibodies, “growth factor receptor” antibodies and tyrosinekinase inhibitors, such as for example gefitinib, imatinib, lapatinib and trastuzumab); antimetabolites (e.g. antifolates such as methotrexate, raltitrexed, pyrimidine analogues such as 5-fluorouracil, capecitabin and gemcitabin, purine and adenosine analogues such as mercaptopurine, thioguanine, cladribine and pentostatin, cytarabine, fludarabine); antitumour antibiotics (e.g. anthracyclins such as doxorubicin, daunorubicin, epirubicin and idarubicin, mitomycin-C, bleomycin, dactinomycin, plicamycin, streptozocin); platinum derivatives (e.g. cisplatin, oxaliplatin, carboplatin); alkylation agents (e.g. estramustin, meclorethamine, melphalan, chlorambucil, busulphan, dacarbazin, cyclophosphamide, ifosfamide, temozolomide, nitrosoureas such as for example carmustin and lomustin, thiotepa); antimitotic agents (e.g. Vinca alkaloids such as for example vinblastine, vindesin, vinorelbin and vincristine; and taxanes such as paclitaxel, docetaxel); topoisomerase inhibitors (e.g. epipodophyllotoxins such as for example etoposide and etopophos, teniposide, amsacrin, topotecan, irinotecan, mitoxantron) and various chemotherapeutic agents such as amifostin, anagrelid, clodronat, filgrastin, interferon alpha, leucovorin, rituximab, procarbazine, levamisole, mesna, mitotane, pamidronate and porfimer.

Suitable preparations include for example tablets, capsules, suppositories, solutions,—particularly solutions for injection (s.c., i.v., i.m.) and infusion—elixirs, emulsions or dispersible powders. The content of the pharmaceutically active compound(s) should be in the range from 0.1 to 90 wt.-%, preferably 0.5 to 50 wt.-% of the composition as a whole, i.e. in amounts which are sufficient to achieve the dosage range specified below. The doses specified may, if necessary, be given several times a day.

Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number of layers to achieve delayed release, possibly using the excipients mentioned above for the tablets.

Syrups or elixirs containing the active substances or combinations thereof according to the invention may additionally contain a sweetener such as saccharine, cyclamate, glycerol or sugar and a flavour enhancer, e.g. a flavouring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions for injection and infusion are prepared in the usual way, e.g. with the addition of isotonic agents, preservatives such as p-hydroxybenzoates, or stabilisers such as alkali metal salts of ethylenediamine tetraacetic acid, optionally using emulsifiers and/or dispersants, whilst if water is used as the diluent, for example, organic solvents may optionally be used as solvating agents or dissolving aids, and transferred into injection vials or ampoules or infusion bottles.

Capsules containing one or more active substances or combinations of active substances may for example be prepared by mixing the active substances with inert carriers such as lactose or sorbitol and packing them into gelatine capsules.

Suitable suppositories may be made for example by mixing with carriers provided for this purpose, such as neutral fats or polyethyleneglycol or the derivatives thereof.

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose) emulsifiers (e.g. lignin, spent sulphite liquors, methylcellulose, starch and polyvinylpyrrolidone) and lubricants (e.g. magnesium stearate, talc, stearic acid and sodium lauryl sulphate).

The preparations are administered by the usual methods, preferably by oral or transdermal route, most preferably by oral route. For oral administration the tablets may, of course contain, apart from the abovementioned carriers, additives such as sodium citrate, calcium carbonate and dicalcium phosphate together with various additives such as starch, preferably potato starch, gelatine and the like. Moreover, lubricants such as magnesium stearate, sodium lauryl sulphate and talc may be used at the same time for the tabletting process. In the case of aqueous suspensions the active substances may be combined with various flavour enhancers or colourings in addition to the excipients mentioned above.

For parenteral use, solutions of the active substances with suitable liquid carriers may be used. The dosage for intravenous use is from 1-1000 mg per hour, preferably between 5 and 500 mg per hour.

However, it may sometimes be necessary to depart from the amounts specified, depending on the body weight, the route of administration, the individual response to the drug, the nature of its formulation and the time or interval over which the drug is administered. Thus, in some cases it may be sufficient to use less than the minimum dose given above, whereas in other cases the upper limit may have to be exceeded. When administering large amounts it may be advisable to divide them up into a number of smaller doses spread over the day.

The formulation examples which follow illustrate the present invention without restricting its scope:

### Examples of Pharmaceutical Formulations

| A) | Tablets | per tablet |
|---|---|---|
| | active substance | 100 mg |
| | lactose | 140 mg |
| | corn starch | 240 mg |
| | polyvinylpyrrolidone | 15 mg |
| | magnesium stearate | 5 mg |
| | | 500 mg |

The finely ground active substance, lactose and some of the corn starch are mixed together. The mixture is screened, then moistened with a solution of polyvinylpyrrolidone in water, kneaded, wet-granulated and dried. The granules, the remaining corn starch and the magnesium stearate are screened and mixed together. The mixture is compressed to produce tablets of suitable shape and size.

| B) | Tablets | per tablet |
|---|---|---|
| | active substance | 80 mg |
| | lactose | 55 mg |
| | corn starch | 190 mg |
| | microcrystalline cellulose | 35 mg |
| | polyvinylpyrrolidone | 15 mg |
| | sodium-carboxymethyl starch | 23 mg |
| | magnesium stearate | 2 mg |
| | | 400 mg |

The finely ground active substance, some of the corn starch, lactose, microcrystalline cellulose and polyvinylpyrrolidone are mixed together, the mixture is screened and worked with the remaining corn starch and water to form a granulate which is dried and screened. The sodiumcarboxymethyl starch and the magnesium stearate are added and mixed in and the mixture is compressed to form tablets of a suitable size.

| C) | Ampoule solution | |
|---|---|---|
| | active substance | 50 mg |
| | sodium chloride | 50 mg |
| | water for inj. | 5 ml |

The active substance is dissolved in water at its own pH or optionally at pH 5.5 to 6.5 and sodium chloride is added to make it isotonic. The solution obtained is filtered free from pyrogens and the filtrate is transferred under aseptic conditions into ampoules which are then sterilised and sealed by fusion. The ampoules contain 5 mg, 25 mg and 50 mg of active substance.

What is claimed is:

1. A compound of formula (1), 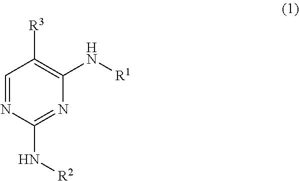
wherein
R¹ denotes a group, substituted by R⁵ and optionally by one or more R⁴, selected from among C₃₋₁₀-Cycloalkyl and 3-8-membered heterocycloalkyl;
R² denotes a group, optionally substituted by one or more R⁴, selected from among C₁₋₆-alkyl, C₃₋₁₀-Cycloalkyl, 3-8-membered heterocycloalkyl, C₆₋₁₅aryl and 5-12-membered heteroaryl;
R³ denotes a group selected from among hydrogen, halogen, —CN, —NO₂, C₁₋₄alkyl, C₁₋₄haloalkyl, C₃₋₁₀-cycloalkyl, C₄₋₁₆-cycloalkylalkyl and C₇₋₁₆arylalkyl;
R⁴ denotes a group selected from among R^{a}, R^{b} and R^{a} substituted by one or more identical or different R^{c} and/or R^{b};
R⁵ denotes a group selected from among —C(O)R^{c}, —C(O)NR^{c}R^{c}, —S(O)₂R^{c}, —N(R^{f})S(O)₂R^{c}, —N(R^{f})C(O)R^{c}, —N(R^{f})C(O)OR^{c}, and —N(R^{f})C(O)NR^{c}R^{c};
each R^{a} is selected independently of one another from among C₁₋₆alkyl, C₃₋₁₀-cycloalkyl, C₄₋₁₆-cycloalkylalkyl, C₆₋₁₀aryl, C₇₋₁₆arylalkyl, 2-6-membered heteroalkyl, 3-8-membered heterocycloalkyl, 4-14-membered heterocycloalkylalkyl, 5-12-membered heteroaryl and 6-18-membered heteroarylalkyl;
each R^{b} is a suitable group and each selected independently of one another from among ═O, —OR^{c}, C₁₋₃haloalkyloxy, —OCF₃, ═S, —SR^{c}, ═NR^{c}, ═NOR^{c}, —NR^{c}R^{c}, halogen, —CF₃, —CN, —NC, —OCN, —SCN, —NO₂, —S(O)R^{c}, —S(O)₂R^{c}, —S(O)₂OR^{c}, —S(O)NR^{c}R^{c}, —S(O)₂NR^{c}R^{c}, —OS(O)R^{c}, —OS(O)₂R^{c}, —OS(O)₂OR^{c}, —OS(O)₂NR^{c}R^{c}, —C(O)R^{c}, —C(O)OR^{c}, —C(O)NR^{c}R^{c}, —CN(R^{f})NR^{c}R^{c}, —CN(OH)R^{c}, —CN(OH)NR^{c}R^{c}, —OC(O)R^{c}, —OC(O)OR^{c}, —OC(O)NR^{c}R^{c}, —OCN(R^{f})NR^{c}R^{c}, —N(R^{f})C(O)R^{c}, —N(R^{f})C(S)R^{c}, —N(R^{f})S(O)₂R^{c}, —N(R^{f})C(O)OR^{c}, —N(R^{f})C(O)NR^{c}R^{c}, —[N(R^{f})C(O)]₂R^{c}, —N[C(O)]₂R^{c}, —N[C(O)]₂OR^{c}, —[N(R^{f})C(O)]₂OR^{c} and —N(R^{f})CN(R^{f})NR^{c}R^{c};
each R^{c} independently of one another is hydrogen or a group optionally substituted by one or more identical or different R^{d} and/or R^{e} selected from among C₁₋₆alkyl, C₃₋₁₀-cycloalkyl, C₄₋₁₁-cycloalkylalkyl, C₆₋₁₀aryl, C₇₋₁₆ arylalkyl, 2-6-membered heteroalkyl, 3-8-membered heterocycloalkyl, 4-14-membered heterocycloalkylalkyl, 5-12-membered heteroaryl and 6-18-membered heteroarylalkyl,
each R^{d} independently of one another is hydrogen or a group optionally substituted by one or more identical or different R^{e} and/or R^{f} selected from among C₁₋₆alkyl, C₃₋₈-cycloalkyl, C₄₋₁₁-cycloalkylalkyl, C₆₋₁₀aryl, C₇₋₁₆arylalkyl, 2-6-membered heteroalkyl, 3-8-membered heterocycloalkyl, 4-14-membered heterocycloalkylalkyl, 5-12-membered heteroaryl and 6-18-membered heteroarylalkyl;
each R^{e} is a suitable group and each selected independently of one another from among ═O, —OR^{f}, C₁₋₃haloalkyloxy, —OCF₃, ═S, —SR^{f}, ═NR^{f}, ═NOR^{f}, —NR^{f}R^{f}, halogen, —CF₃, —CN, —NC, —OCN, —SCN, —NO₂, —S(O)R^{f}, —S(O)₂R^{f}, —S(O)₂OR^{f}, —S(O)NR^{f}R^{f}, —S(O)₂NR^{f}R^{f}, —OS(O)R^{f}, —OS(O)₂R^{f}, —OS(O)₂OR^{f}, —OS(O)₂NR^{f}R^{f}, —C(O)R^{f}, —C(O)OR^{f}, —C(O)NR^{f}R^{f}, —CN(R^{g})NR^{f}R^{f}, —CN(OH)R^{f}, —C(NOH)NR^{f}R^{f}, —OC(O)R^{f}, —OC(O)OR^{f}, —OC(O)NR^{f}R^{f}, —OCN(R^{g})NR^{f}R^{f}, —N(R^{g})C(O)R^{f}, —N(R^{g})C(S)R^{f}, —N(R^{g})S(O)₂R^{f}, —N(R^{d})C(O)OR^{f}, —N(R^{g})C(O)NR^{f}R^{f}, and —N(R^{g})CN(R^{f})NR^{f}R^{f};
each R^{f} independently of one another is hydrogen or a group optionally substituted by one or more identical or different R^{g} selected from among C₁₋₆alkyl, C₃₋₈-cycloalkyl, C₄₋₁₁-cycloalkylalkyl, C₆₋₁₀aryl, C₇₋₁₆arylalkyl, 2-6-membered heteroalkyl, 3-8-membered heterocycloalkyl, 4-14-membered heterocycloalkylalkyl, 5-12-membered heteroaryl and 6-18-membered heteroarylalkyl;
each R^{g} independently of one another is hydrogen, C₁₋₆alkyl, C₃₋₈-cycloalkyl, C₄₋₁₁-cycloalkylalkyl, C₆₋₁₀aryl, C₇₋₁₆arylalkyl, 2-6-membered heteroalkyl, 3-8-membered heterocycloalkyl, 4-14-membered heterocycloalkyl, 5-12-membered heteroaryl and 6-18-membered heteroarylalkyl;
optionally in the form of a tautomer, a racemate, an enantiomer, a diastereomer or a mixture thereof, or a pharmacologically acceptable acid addition salt thereof.

2. The compound according to claim 1, wherein R³ denotes a group selected from among halogen and C₁₋₄haloalkyl.

3. The compound according to claim 2, wherein R³ denotes —CF₃.

4. The compound according to claim 1, wherein R² denotes C₆₋₁₀aryl or 5-12-membered heteroaryl, optionally substituted by one or more R⁴.

5. The compound according to claim 4, wherein R² denotes phenyl, optionally substituted by one or more R⁴.

6. The compound according to claim 1 of general formula (1A), 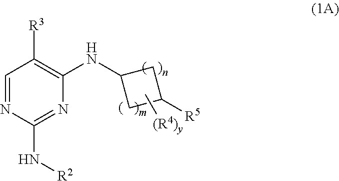
wherein
n is equal to 0 or 1, and
m is equal to 1-5, and
y is equal to 0 to 6.

7. The compound according to claim 6, wherein R³ denotes a group selected from among halogen and C₁₋₄haloalkyl.

8. The compound according to claim 7, wherein R³ denotes CF₃.

9. The compound according to claim 6, wherein R² denotes C₆₋₁₀aryl or 5-12-membered heteroaryl, optionally substituted by one or more R⁴.

10. The compound according to claim 6, wherein R² denotes phenyl, optionally substituted by one or more R⁴.

11. A pharmaceutical preparation comprising as active substance one or more compounds of formula (1) according to claim 1 and one or more excipients or carriers.

12. A pharmaceutical preparation comprising as active substance a compound of formula (1) according to claim 1 and at least one other cytostatic or cytotoxic active substance different from the compound of formula (1).

13. A pharmaceutical preparation comprising as active substance one or more compounds of formula (1A) according to claim 6 and one or more excipients or carriers.

14. A pharmaceutical preparation comprising as active substance a compound of formula (1A) according to claim 6 and at least one other cytostatic or cytotoxic active substance different from the compound of formula (1A).


SEQUENCE LISTING (4 sequences)

SEQ ID NO: 1 - protein, 13 aa, Artificial Sequence
  Description of Artificial Sequence Synthetic peptide
EPLERRLSLVPDS

SEQ ID NO: 2 - protein, 13 aa, Artificial Sequence
  Description of Artificial Sequence Synthetic peptide
EPLERRLSLVPKM

SEQ ID NO: 3 - protein, 28 aa, Artificial Sequence
  Description of Artificial Sequence Synthetic peptide
LRRWSLGLRRWSLGLRRWSLGLRRWSLG

SEQ ID NO: 4 - protein, 6 aa, Artificial Sequence
  Description of Artificial Sequence Synthetic 6xHis tag
HHHHHH